SEQUENCE LISTING (24 sequences)

SEQ ID NO: 1 - protein, 1362 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 2 - DNA, 5198 bp, Homo sapiens
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaaggaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctgagcgcacctaggtggcttctgactttgattttctggcaaaacattgactttccatagtgttaggggcggtggtggaggtgggatcagcggccaggggatgcctcagggcctggccctcctgcatgctatgcccggggcaggcctgacgggcagctgaggattgcagagcctgtctgccttacggccagtcggacagacgtcccgccacccaccacccctcacaggacgtccgctcagcacacgccttgttacgagcaagtgccggctggacccaagccctgcatccccacatgcggggcagaggcccttctctccgccaaatgtctacacagtatacacaggacatcgttgctgccgccgtgactggttttctgtccccaagaacgtgacgttcgtgatgtcctgcccgccgggagtctttccccacaccccagccatcgccgcccgctcccaggaggccagggcaggcctgcgtgggctggaggcgggcgaggccggcccaccccctcgctggcactgactttgccttgaacagaccccccgaccctcccccacaagcctttaattgagagccgctctctgtaagtgtttgcttgtgcaaaagggaatagtgccgtggaggtgtgtgtgtccatggcatccggagcgaggcgactgtcctgcgtgggtagccctcggccggggagtgaggccaccaaccaaagtcagttccttcccacctgtgtttctgtttcgtttttttttttcttttttttctatatatattttttgttgaattctattttatttttaattctctcttctcctccagacacaatggcactgcttatctccgaaatggtgtgatcgtctcctcattgagcagcggctgccaccgcgctgtgggta

SEQ ID NO: 3 - protein, 726 aa, Homo sapiens
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRQLKKGGKQASASYDSEEEEEGLPMSYDEKRQLSLDINRLPGEKLGRVVHIIQSREPSLRDSNPDEIEIDFETLKPTTLRELERYVKSCLQKKQRKPFSASGKKQAAKSKEELAQEKKKELEKRLQDVSGQLSSSKKPARKEKPGSAPSGGPSRLSSSSSSESGSSSSSGSSSDSSDSE

SEQ ID NO: 4 - DNA, 3242 bp, Homo sapiens
aggctaattaggcgcgcggtttgtttacaaacacgggctcccggcaggtgcgcgccgccccgcccgtgcgcggccggggttcgagggtggctcccgcgggcctcggggtgcccggacgggggctgcggtgctggctgcgtgcccgcttcttccatgccgtcctggggcaccggaaaatccgccgccaggcgctgtccccgacacgggctgtcgcctggttgggcccggaaatgggacgtcgcgctttctcagggagcgtagaagcagccagggcctctccaagccgctgctgtgacagaaagtgagtgagctgccggaggatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctacgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccccgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagacagctgaagaaaggcggcaagcaggcatctgcctcctacgactcagaggaagaggaggagggcctgcccatgagctacgatgaaaagcgccagcttagcctggacatcaaccggctgcccggggagaagctgggccgggtagtgcacatcatccaatctcgggagccctcgctcagggactccaaccccgacgagatagaaattgactttgagactctgaaacccaccactttgcgggaactggagagatatgtcaagtcttgtttacagaaaaagcaaaggaaaccgttctcagcaagcgggaagaaacaggcagccaagtcgaaagaggagctagctcaggaaaagaagaaggagctggaaaagcgtctgcaggatgtcagcgggcagctgagcagcagcaagaagcccgcccggaaagagaagcccggctcagcaccctcagggggcccgtccaggctcagcagcagcagctcctccgagtctgggagcagcagctccagcgggtccagctctgacagcagtgactcagaatgaactggcttcggacagaacaggacagatggatgtcgcacacgccgagactctgccgtacccctctgtggttcatattactacttctgttccatggtgtgcaggtctgcttcttaattcagtgttatgatatcttccagtttttgctttcataggtcagagatctatcttgtgtgtgcgttagacttgatgagaaggtgtgaactctgcagaaagtctcttcttcatcactgaattcagtcacttggagatgacaacttcaaatgctaacccgatgaccccagaaaaccgtgtgagattcgtaccgaagaaccttgtggaatccctttgcttaggcccaacctggtcgatagctcgagaaagaattttttccaaggaaatgtctcggatatgggtactgtatttgaaagctgttagctttgtcaacacgcattgtccttgtcatttgggccccgagctctgaccctcgtgtctgacgcggccacctctttctggaggggctgaggacagatgtgcctgcttgtggagaccaggctgggcctaagcgaagggtcatcgcagccccagcccggagcgtggagcccttggggggtggtcgggtgggatgtgcgttctccgctcgtggtgatgtcaggagctcctcggagggaacagagcggctgtgtatgcagcctgcaggtttccatacactgaagcttttacctcaactttaaaaaaaaaaaaagaagaaaagatttaaaaaaa

SEQ ID NO: 5 - protein, 801 aa, Homo sapiens
MLQNVTPHNKLPGEGNAGLLGLGPEAAAPGKRIRKPSLLYEGFESPTMASVPALQLTPANPPPPEVSNPKKPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMPQEEQELVVTIPKNSHKKGAKLAALQGSVTSAHQVPAVSSVSHTALYTPPPEIPTTVLNIPHPSVISSPLLKSLHSAGPPLLAVTAAPPAQPLAKKKGVKRKADTTTPTPTAILAPGSPASPPGSLEPKAARLPPMRRESGRPIKPPRKDLPDSQQQHQSSKKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMPDEPLEPGPLPVSTAMPPGLAKSSSESSSEESSSESSSEEEEEEDEEDEEEEESESSDSEEERAHRLAELQEQLRAVHEQLAALSQGPISKPKRKREKKEKKKKRKAEKHRGRAGADEDDKGPRAPRPPQPKKSKKASGSGGGSAALGPSGFGPSGGSGTKLPKKATKTAPPALPTGYDSEEEEESRPMSYDEKRQLSLDINKLPGEKLGRVVHIIQAREPSLRDSNPEEIEIDFETLKPSTLRELERYVLSCLRKKPRKPYTIKKPVGKTKEELALEKKRELEKRLQDVSGQLNSTKKPPKKANEKTESSSAQQVAVSRLSASSSSSDSSSSSSSSSSSDTSDSDSG

SEQ ID NO: 6 - DNA, 4761 bp, Homo sapiens
gctcgccctcctccctggcgctgaccgatggaccagccgctccgtggggaggactccggaccctggtgggggggcgggggggttctttcgcccccgtggcggagggcccctgagaggcggatacgggtgtgcctttgggggtgatgtggcgtgtggggggaaaggtccgagctcgcctggagggggagggtttttcccttaagtcatccctcccaggacttgctttttctgctctgagccggacgccggaatggagtttgaggaagagattcttcgctgctgctgccttaccgccgagaaccaccacccgccaggcgtcttgcggccacacccctggcgggttcaggcaggctacgcccacgcgacccctcccgtttccctgctttggccaatggaggagctacgaatggcacgacctgctcgagcttggcagtctccagttgggctgtgcatggaagcttgggaagactttgttggaaggggaggcggggagagagtgctggaggctctggggcgatggcttccgcacctcttccaaccaccctctttccctggagtcggcggaccacagctcagccaattggcttggagatgtggcgggttgccacttccctgtgggtctctgcggcactcttctgcctggtgactgacaccttggaaatgaagtttatgacgtcatcgttgcggctggccaatagaaaaagctcccgcggagaggtgttccttccccttcgactcagcttcttcacccgcgtgagcgagcgcgcgcgcgcggagggggtggggaaaagctcaagcagggtggcgcgcatgagcggcgaagctcctcctccccgcctatatataaagggctggcgcggggctcggcggcgccatttcgtgctggagtggagcagcctctagaacgagctggaggattctgcctaccgatacagagccttcgagtcgtccggggccgccattacaatccacctccatccgcttggaaatggccttcgtcccggcctatgactggtcccagcgggcagtacagaccccctagaagcccctggagctcccctttttcgggccccgcccaatcctcggagtctgtccaccccctctactccgccctcaagaggatttcaaagatggaggcggcggctccctaaaccacttttcgtgttcatccgcctccatccgagatcgaaacgggacctcgtcggccccgtaggggcccgacaagaagagggaatccctgcagaccaacagcgggctatattgacgacggtgtctgagatcggggaccgtcttttgaagagtcagtccctccttagttgcccgcctcagctgaggccgccgccattttcttgctgtccgccgtctgcagagcgcgccaagctgcccggagctctccgagaggccccaaagagactgctttcgtgccggccaggcagggggtttgtcgcctggaggcccaagaggaacggcctccccccaacttagcgggttatgctggaccgggcggtgaggggaaccgaggccacccggactttccgcggctgagggcagcgccggttccttgcggtcaagatgctgcaaaacgtgactccccacaataagctccctggggaagggaatgcagggttgctggggctgggcccagaagcagcagcaccagggaagaggattcgaaaaccctctctcttgtatgagggctttgagagccccacaatggcttcggtgcctgctttgcaacttacccctgccaacccaccacccccggaggtgtccaatcccaaaaagccaggacgagttaccaaccagctgcaatacctacacaaggtagtgatgaaggctctgtggaaacatcagttcgcatggccattccggcagcctgtggatgctgtcaaactgggtctaccggattatcacaaaattataaaacagcctatggacatgggtactattaagaggagacttgaaaacaattattattgggctgcttcagagtgtatgcaagattttaataccatgttcaccaactgttacatttacaacaagcccactgatgatattgtcctaatggcacaaacgctggaaaagatattcctacagaaggttgcatcaatgccacaagaagaacaagagctggtagtgaccatccctaagaacagccacaagaagggggccaagttggcagcgctccagggcagtgttaccagtgcccatcaggtgcctgccgtctcttctgtgtcacacacagccctgtatactcctccacctgagatacctaccactgtcctcaacattccccacccatcagtcatttcctctccacttctcaagtccttgcactctgctggacccccgctccttgctgttactgcagctcctccagcccagccccttgccaagaaaaaaggcgtaaagcggaaagcagatactaccacccctacacctacagccatcttggctcctggttctccagctagccctcctgggagtcttgagcctaaggcagcacggcttccccctatgcgtagagagagtggtcgccccatcaagcccccacgcaaagacttgcctgactctcagcaacaacaccagagctctaagaaaggaaagctttcagaacagttaaaacattgcaatggcattttgaaggagttactctctaagaagcatgctgcctatgcttggcctttctataaaccagtggatgcttctgcacttggcctgcatgactaccatgacatcattaagcaccccatggacctcagcactgtcaagcggaagatggagaaccgtgattaccgggatgcacaggagtttgctgctgatgtacggcttatgttctccaactgctataagtacaatcccccagatcacgatgttgtggcaatggcacgaaagctacaggatgtatttgagttccgttatgccaagatgccagatgaaccactagaaccagggcctttaccagtctctactgccatgccccctggcttggccaaatcgtcttcagagtcctccagtgaggaaagtagcagtgagagctcctctgaggaagaggaggaggaagatgaggaggacgaggaggaagaagagagtgaaagctcagactcagaggaagaaagggctcatcgcttagcagaactacaggaacagcttcgggcagtacatgaacaactggctgctctgtcccagggtccaatatccaagcccaagaggaaaagagagaaaaaagagaaaaagaagaaacggaaggcagagaagcatcgaggccgagctggggccgatgaagatgacaaggggcctagggcaccccgcccacctcaacctaagaagtccaagaaagcaagtggcagtgggggtggcagtgctgctttaggcccttctggctttggaccttctggaggaagtggcaccaagctccccaaaaaggccacaaagacagccccacctgccctgcctacaggttatgattcagaggaggaggaagagagcaggcccatgagttacgatgagaagcggcagctgagcctggacatcaacaaattacctggggagaagctgggccgagttgtgcatataatccaagccagggagccctctttacgtgattcaaacccagaagagattgagattgattttgaaacactcaagccatccacacttagagagcttgagcgctatgtcctttcctgcctacgtaagaaaccccggaagccctacaccattaagaagcctgtgggaaagacaaaggaggaactggctttggagaaaaagcgggaattagaaaagcggttacaagatgtcagcggacagctcaattctactaaaaagccccccaagaaagcgaatgagaaaacagagtcatcctctgcacagcaagtagcagtgtcacgccttagcgcttccagctccagctcagattccagctcctcctcttcctcgtcgtcgtcttcagacaccagtgattcagactcaggctaaggggtcaggccagatggggcaggaaggctccgcaggaccggacccctagaccaccctgccccacctgccccttccccctttgctgtgacacttcttcatctcacccccccccgcccccctctaggagagctggctctgcagtgggggagggatgcagggacatttactgaaggagggacatggacaaaacaacattgaattcccagccccattggggagtgatctcttggacacagagcccccattcaaaatggggcagggcaagggtgggagtgtgcaaagccctgatctggagttacctgaggccacagctgccctattcacttctaagggccctgttttgagattgtttgttctaatttattttaagctaggtaaggctggggggagggtggggccgtggtcccctcagcctccatggggagggaagaagggggagctctttttttacgttgatttttttttttctactctgttttccctttttccttccgctccatttggggccctgggggtttcagtcatctccccatttggtcccctggactgtctttgttgattctaacttgtaaataaagaaaatattattcaagttttgagttaccttaatatttgcttttgtagtgtttcaaaaggaacatcataagaattgtcttgataattttgagggaaatattactgcagtgagaaaaggcaatagctaacctataattggattgtcttaatttttaaaccagtaggcttttgctgtgtttttaataaagtaaatatgacttttgtaaa

SEQ ID NO: 7 - protein, 947 aa, Homo sapiens
MSLPSRQTAIIVNPPPPEYINTKKNGRLTNQLQYLQKVVLKDLWKHSFSWPFQRPVDAVKLQLPDYYTIIKNPMDLNTIKKRLENKYYAKASECIEDFNTMFSNCYLYNKPGDDIVLMAQALEKLFMQKLSQMPQEEQVVGVKERIKKGTQQNIAVSSAKEKSSPSATEKVFKQQEIPSVFPKTSISPLNVVQGASVNSSSQTAAQVTKGVKRKADTTTPATSAVKASSEFSPTFTEKSVALPPIKENMPKNVLPDSQQQYNVVKTVKVTEQLRHCSEILKEMLAKKHFSYAWPFYNPVDVNALGLHNYYDVVKNPMDLGTIKEKMDNQEYKDAYKFAADVRLMFMNCYKYNPPDHEVVTMARMLQDVFETHFSKIPIEPVESMPLCYIKTDITETTGRENTNEASSEGNSSDDSEDERVKRLAKLQEQLKAVHQQLQVLSQVPFRKLNKKKEKSKKEKKKEKVNNSNENPRKMCEQMRLKEKSKRNQPKKRKQQFIGLKSEDEDNAKPMNYDEKRQLSLNINKLPGDKLGRVVHIIQSREPSLSNSNPDEIEIDFETLKASTLRELEKYVSACLRKRPLKPPAKKIMMSKEELHSQKKQELEKRLLDVNNQLNSRKRQTKSDKTQPSKAVENVSRLSESSSSSSSSSESESSSSDLSSSDSSDSESEMFPKFTEVKPNDSPSKENVKKMKNECIPPEGRTGVTQIGYCVQDTTSANTTLVHQTTPSHVMPPNHHQLAFNYQELEHLQTVKNISPLQILPPSGDSEQLSNGITVMHPSGDSDTTMLESECQAPVQKDIKIKNADSWKSLGKPVKPSGVMKSSDELFNQFRKAAIEKEVKARTQELIRKHLEQNTKELKASQENQRDLGNGLTVESFSNKIQNKCSGEEQKEHQQSSEAQDKSKLWLLKDRDLARQKEQERRRREAMVGTIDMTLQSDIMTMFENNFD

SEQ ID NO: 8 - DNA, 3430 bp, Homo sapiens
tataaaagcgcctgtcgcgcgaccgccattacaacaaaaactggcggcgaggaactgcggagaactgttgccctgcaccgcttcattttgtgcagcctgaaaggggcaatcacataaggtcggttttgtaatatacaggtaacatacaggtaatgtaccctccacagggggtgctgccttaggccggccccgaggtgcggccttgggtgggaacaaccgttttttcccccctctcccctccccgaccccatcaacatcgtgcgacagtccgggcagttgagaaggccaccccgcttcgtgcctgcagcccagttcctcttaggtcttggactggtgtggatcgctgctgcctgtactgtaattttggaaatgcaggagaaagacatgtacttgtagacaggattcaaagcagttaagaatgtctctgccaagtcgacaaacagctattattgttaaccctcctccaccagaatatataaatactaagaaaaatgggcgattgacaaatcaacttcagtatctacaaaaagttgtcctaaaggatttatggaagcatagtttttcatggccctttcaacgtcctgtggatgctgtgaaactacagttgcctgattattataccattataaaaaacccaatggatttaaatacaattaagaagcgcttggagaataaatattatgcgaaggcttcagaatgtatagaagacttcaatacaatgttctcaaattgttatttatataacaagcctggagatgacattgttcttatggcacaagctctagagaagctgtttatgcagaaattatctcagatgccacaagaagagcaagttgtgggtgttaaggaaagaatcaagaaaggcactcaacagaatatagctgtttcttctgctaaagaaaaatcatcacccagcgcaacagaaaaagtatttaagcagcaagaaattccttctgtatttcctaagacatctatttctcccttgaacgtggtacagggagcttcagtcaactccagttcacaaactgcggcccaagttacaaaaggtgtgaagaggaaagcagatacaacaactcctgcaacttcagcagttaaagcaagtagtgaattttctccaacattcacagaaaaatcagtggcactgccacctataaaagaaaatatgccaaagaatgttttgccagattctcagcaacaatataatgttgtgaagactgttaaagtaactgaacaattaaggcactgtagtgagattcttaaagaaatgcttgcaaagaaacatttttcatatgcatggcccttttataatcctgttgacgttaatgctttgggactccataactactatgacgttgtcaaaaatccgatggatcttggaactattaaggagaaaatggataaccaagaatataaggatgcatacaaatttgcggcagatgttagattaatgttcatgaattgctacaagtacaatcctccagatcacgaagttgtgacaatggcaagaatgcttcaggatgttttcgaaacgcatttttcaaagatcccgattgaacctgttgagagtatgcctttatgttacatcaaaacagatatcacagaaaccactggtagagagaacactaatgaagcctcctctgaagggaactcttctgatgattctgaagatgagcgagttaagcgtcttgcaaagcttcaggagcagcttaaagctgtacatcaacagctccaggttttgtcccaagtacctttccgtaagctaaataaaaagaaagagaagtctaaaaaggaaaagaaaaaagaaaaggttaataacagcaatgaaaatccaagaaaaatgtgtgagcaaatgaggctaaaggaaaagtccaagagaaatcagccaaagaaaaggaaacaacagttcattggtctaaaatctgaagatgaagataatgctaaacctatgaactatgatgagaaaaggcagttaagtctgaatataaacaaactccctggagataaacttgggcgagtagttcacataatacaatcaagagagccttctctgagcaattccaatcctgatgagatagagatagactttgaaacactgaaagcatcaacactaagagaattagaaaaatatgtttcggcatgtctaagaaagagaccattaaaacctcctgctaagaaaataatgatgtccaaagaagaacttcactcacagaaaaaacaggaattggaaaagcggttactggatgttaataatcagttaaattctagaaaacgtcaaacaaaatctgataaaacgcaaccatccaaagctgttgaaaatgtttcccgactgagtgagagcagcagcagcagcagcagctcatcagagtctgaaagtagcagcagtgacttaagctcttcagacagcagtgattctgaatcagaaatgttccctaagtttacagaagtaaaaccaaatgattctccttctaaagagaatgtaaagaaaatgaagaatgaatgcataccgcctgaaggaagaacaggcgtcacacagataggatattgtgtgcaagacacaacctctgccaatactacccttgttcatcagaccacaccttcacatgtaatgccaccaaatcaccaccaattagcatttaattatcaagaattagaacatttacagactgtgaaaaacatttcacctttacaaattctgcctccctcaggtgattctgaacagctctcaaatggcataactgtgatgcatccatctggtgatagtgacacaacgatgttagaatctgaatgtcaagctcctgtacagaaggatataaagattaagaatgcagattcatggaaaagtttaggcaaaccagtgaaaccatcaggtgtaatgaaatcctcagatgagctcttcaaccaatttagaaaagcagccatagaaaaggaagtaaaagctcggacacaggaactcatacggaagcatttggaacaaaatacaaaggaactaaaagcatctcaagaaaatcagagggatcttgggaatggattgactgtagaatctttttcaaataaaatacaaaacaagtgctctggagaagagcagaaagaacatcagcagtcatcagaagctcaagataaatccaaactctggcttctcaaagaccgtgatttagcaaggcagaaagaacaagagaggaggaggagagaagcaatggtgggtaccattgatatgacccttcaaagtgacattatgacaatgtttgaaaacaactttgattaaaactcagtttttaaattaaccatcaacttaaaatgaatggtaaaagatcaaaatgcatatggtaaaatgattgctttcagataacaagataccaatcttatattgtattttgactgctctaaaatgattaaacagttttcacttacatttttaaaaaaaaaaaaaaaa

SEQ ID NO: 9 - protein, 125 aa, Artificial Sequence
  Synthetic Polypeptide
NPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEE

SEQ ID NO: 10 - protein, 125 aa, Artificial Sequence
  Synthetic Polypeptide
NPPPPETSNPNKPKRQTNQLQYLLRVVLKTLWKHQFAWPFQQPVDAVKLNVPDYYKIIKTPMDMGTIKKRLENNYYWNAQECIQDFNTMFTNCYIYNKPGDDIVLMAEALEKLFLQKINELPTEE

SEQ ID NO: 11 - protein, 128 aa, Artificial Sequence
  Synthetic Polypeptide
KDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDE

SEQ ID NO: 12 - protein, 128 aa, Artificial Sequence
  Synthetic Polypeptide
KDVPDSQQHPAPEKSSKVSEQLKCCSGILKEMFAKKHAAYAWPFYKPVDVEALGVHDYCDIIKHPMDMSTIKSKLEAREYRDAQEFGADVRLMFSNCYKYNPPDHEVVAMARKLQDVFEMRFAKMPDE

SEQ ID NO: 13 - protein, 114 aa, Artificial Sequence
  Synthetic Polypeptide
KPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMPQEE

SEQ ID NO: 14 - protein, 114 aa, Artificial Sequence
  Synthetic Polypeptide
KPGRVTNQLQYLHKVVMKALWKHQFAWPFRQPVDAVKLGVPDYHKIIKQPMDMGTIKRRLENNYYWAASECMQDFNTMFTNCYIYNKPTDDIVLMAQTLEKIFLQKVASMPQEE

SEQ ID NO: 15 - protein, 112 aa, Artificial Sequence
  Synthetic Polypeptide
GKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMPD

SEQ ID NO: 16 - protein, 112 aa, Artificial Sequence
  Synthetic Polypeptide
GKLSEQLKHCNGILKELLSKKHAAYAWPFYKPVDASALGVHDYHDIIKHPMDLSTVKRKMENRDYRDAQEFAADVRLMFSNCYKYNPPDHDVVAMARKLQDVFEFRYAKMPD

SEQ ID NO: 17 - protein, 121 aa, Artificial Sequence
  Synthetic Polypeptide
PEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEE

SEQ ID NO: 18 - protein, 121 aa, Artificial Sequence
  Synthetic Polypeptide
PEVSNPSKPGRKTNQLQYMQNVVVKTLWKHQFAWPFYQPVDAIKLNVPDYHKIIKNPMDMGTIKKRLENNYYWSASECMQDFNTMFTNCYIYNKPTDDIVLMAQALEKIFLQKVAQMPQEE

SEQ ID NO: 19 - protein, 111 aa, Artificial Sequence
  Synthetic Polypeptide
GKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMP

SEQ ID NO: 20 - protein, 111 aa, Artificial Sequence
  Synthetic Polypeptide
GKLSEHLRYCDSILREMLSKKHAAYAWPFYKPVDAEALEVHDYHDIIKHPMDLSTVKRKMDGREYPDAQGFAADVRLMFSNCYKYNPPDHEVVAMARKLQDVFEMRFAKMP

SEQ ID NO: 21 - protein, 117 aa, Artificial Sequence
  Synthetic Polypeptide
NTKKNGRLTNQLQYLQKVVLKDLWKHSFSWPFQRPVDAVKLQLPDYYTIIKNPMDLNTIKKRLENKYYAKASECIEDFNTMFSNCYLYNKPGDDIVLMAQALEKLFMQKLSQMPQEE

SEQ ID NO: 22 - protein, 117 aa, Artificial Sequence
  Synthetic Polypeptide
NTKKNGRLTNQLQYLQKVVLKDLWKHSFSWPFQRPVDAVKLQVPDYYTIIKNPMDLNTIKKRLENKYYAKASECIEDFNTMFSNCYLYNKPGDDIVLMAQALEKLFMQKLSQMPQEE

SEQ ID NO: 23 - protein, 101 aa, Artificial Sequence
  Synthetic Polypeptide
LRHCSEILKEMLAKKHFSYAWPFYNPVDVNALGLHNYYDVVKNPMDLGTIKEKMDNQEYKDAYKFAADVRLMFMNCYKYNPPDHEVVTMARMLQDVFETHF

SEQ ID NO: 24 - protein, 101 aa, Artificial Sequence
  Synthetic Polypeptide
LRHCSEILKEMLAKKHFSYAWPFYNPVDVNALGVHNYYDVVKNPMDLGTIKEKMDNQEYKDAYKFAADVRLMFMNCYKYNPPDHEVVTMARMLQDVFETHF

(19) United States
(12) Patent Application Publication
(10) Pub. No.: US 2023/0234957 A1
(43) Pub. Date: Jul. 27, 2023

(54) PROTEIN TAG TO INDUCE LIGAND DEPENDENT DEGRADATION OF PROTEIN/PROTEIN-FUSIONS

(71) Applicant: DANA-FARBER CANCER INSTITUTE, INC., Boston, MA (US)

(72) Inventors: Eric Fischer, Newton, MA (US); Radoslaw Nowak, Boston, MA (US); Yuan Xiong, Waltham, MA (US)

(73) Assignee: DANA-FARBER CANCER INSTITUTE, INC., Boston, MA (US)

(21) Appl. No.: 18/001,995

(22) PCT Filed: Jun. 21, 2021
(86) PCT No.: PCT/US2021/038238

§ 371 Date: Dec. 15, 2022

Related U.S. Application Data
(60) Provisional application No. 63/042,257, filed on Jun. 22, 2020.

(51) Int. Cl. C07D 487/04; C07K 14/705; C12N 15/63

(52) U.S. Cl. CPC C07D 487/04; C07K 14/70596; C12N 15/63; C07K 2319/03; C07K 2319/95

(57) ABSTRACT

Disclosed is a dTAG system comprising small molecule degraders of mutant BET family protein-tagged proteins via recruitment of an E3 ubiquitin ligase and uses thereof.

### RELATED APPLICATIONS

This application claims the benefit of priority under 35 U.S.C. § 119(e) to U.S. Provisional Application No. 63/042,257, filed on Jun. 22, 2020, which is incorporated herein by reference in its entirety.

### BACKGROUND OF THE INVENTION

Genetic mechanisms for modulating abundance of gene products such as RNA interference and Clustered Regularly Interspaced Short Palindromic Repeats (CRISPR)-Cas9 genome editing have become powerful tools for determining the consequences of functional loss or gain of a target gene. However, these methodologies are limited from the standpoint of being able to assess acute changes in protein function, particularly with respect to proteins that are required for cell growth and survival.

Chemical-based methodologies have been developed as well. PROteolysis-TArgeting Chimeras (PROTACs) exploit the intracellular ubiquitin-proteasome system to selectively degrade target proteins (Winter et al., Science 348:1376-81(2015), Raina et al., Pharmacol. Ther. 174:138-44 (2017)). Use of these small molecule degraders has been shown to be advantageous in that it allows rapid and target-specific turnover without degradation of the bifunctional degraders. On the other hand, this approach has been found to be limited in that it requires the up-front identification of a ligand that binds the protein that is intended for degradation.

### SUMMARY OF THE INVENTION

A first aspect of the present invention is directed to a bifunctional compound, also referred to herein as a degrader, having a structure represented by formula I:

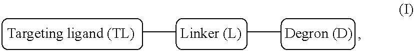

wherein the targeting ligand is represented by any one of structures TL1 to TL3:

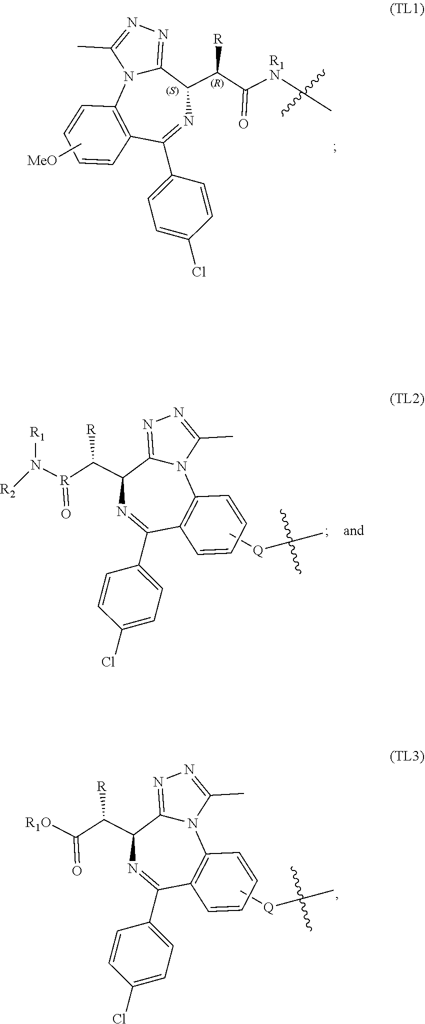

wherein
R represents methyl, ethyl, propyl, isopropyl, or allyl,
R₁ and R₂ independently represent H, methyl, or ethyl, and
Q represents a bond, CH₂, N, or O;
the linker represents a moiety that connects covalently the degron and the targeting ligand; and the degron represents a ligand that binds an E3 ubiquitin ligase, or a pharmaceutically acceptable salt or stereoisomer thereof.

Another aspect of the present invention is directed to a degradation tag (dTAG) comprising a bromodomain and extraterminal domain family protein (e.g., bromodomain-containing proteins 2 (BRD2), BRD3, and BRD4, and bromodomain testis-specific protein (BRDT)), or a bromodomain thereof that includes a BET-ligand binding domain or pocket, wherein the BET-binding pocket contains an amino acid substitution of a conserved Leu residue, wherein the dTAG comprises an amino acid sequence selected from any one of SEQ ID NOs: 9-24.

Yet another aspect of the present invention is directed to a nucleic acid that contains a first fragment encoding the dTAG comprising any one of SEQ ID NOs: 9-24, and a second fragment encoding a protein of interest that is an intended target for selective degradation. A related aspect is directed to a dTAG system that includes the nucleic acid and the bifunctional compound or degrader. When the dTAG and the degrader are brought into contact, the dTAG is bound by the degrader and is brought into close proximity with the degron. The fusion protein, which is the dTAG-target protein, is ubiquitinated and then degraded by endogenous cellular proteasomes.

Other aspects of the present invention are directed to methods of using the inventive bifunctional compounds and dTAGs that they bind, including the inventive dTAGs, for both clinical and pre-clinical purposes. The inventive methods may broadly employ a dTAG that binds any of the inventive bifunctional compounds, wherein the dTAG comprises a BET protein, or a bromodomain thereof containing a BET ligand-binding domain, wherein the BET-ligand binding domain contains a substitution of a conserved Leu residue.

In some embodiments, the method includes genetically modifying a cell by introducing an exogenous nucleic acid having a sequence that encodes a fusion protein comprising a mutated form of a protein that is endogenous to the cell and a dTAG (which in some embodiments is fused in-frame to the N-terminus or the C-terminus of the mutated protein); contacting the modified cells with an inventive bifunctional compound; and determining a change in a property of the modified cell before and after said contacting with the bifunctional compound. In some aspects, the methods include modifying expression of a polynucleotide in a eukaryotic cell by introducing a nucleic acid encoding the dTAG.

In vivo methods may include genetically modifying a cell by introducing an exogenous nucleic acid comprising a sequence that encodes the dTAG at a genetic locus of an endogenous protein, wherein the thus modified locus expresses the protein with the dTAG as an in-frame fusion (e.g., an N-terminal or C-terminal fusion); introducing the thus modified cells into a non-human animal (e.g., a rodent (e.g., a mouse)); administering to the non-human animal a bifunctional compound comprising a targeting ligand that binds the dTAG covalently linked via a linker to a ligand that binds an E3 ubiquitin ligase; and detecting a change in a property of the non-human animal relative to an unmodified non-human animal. The cells may be autologous or non-autologous to the non-human animal. In some embodiments, the cell may be a human cell, e.g., a human cancer cell line or a non-cancerous cell line (for other non-cancerous conditions). In some embodiments, the non-human animal is a rodent such as a mouse.

In some embodiments, the methods may be used clinically, and include administering to a subject (also referred to herein as a “patient”) immune effector cells such as autologous or allogeneic T-cells (CAR-T cells) which have been genetically modified to express a chimeric antigen receptor protein (CAR)-dTAG fusion protein. In the event the patient experiences an adverse immune response (e.g., cytokine release syndrome or neurotoxicity) as a result of the CAR-T therapy, the patient may then be administered the bifunctional targeting compound which will result in degradation of the CAR-dTAG fusion protein.

Further, the dTAG system of the present invention may be used to conduct high-throughput screening studies wherein the degraders function as a benchmark for effective degradation or as a reporter output (e.g., GFP/mCherry). The system may also be used to provide tunable control of Clustered Regularly Interspaced Short Palindromic Repeats (CRISPR) associated protein 9 (Cas9).

### BRIEF DESCRIPTION OF THE DRAWINGS

FIG. 1A-FIG. 1B show an image of the crystal structure of inhibitor 1-2 binding mutant BRD4_{BD1} and a chemical structure of known selective BRD4 degrader ZXH-3-26 (FIG. 1A) (Runcie et al., Chem. Sci. 9:2452-2468 (2018)), and a sequence alignment of BRD2/3/4/T showing the conserved L.

FIG. 2A.-FIG. 2H show quantitative assessment of cellular degradation using a BRD4_{BD1}-EGFP or BRD4_{BD1 L94B (bumped)}-EGFP reporter assays.

FIG. 3A.-FIG. 3H show quantitative assessment of cellular degradation using a BRD4_{BD2}-EGFP reporter assay. Cells stably expressing BRD4_{BD2}-EGFP and mCherry were treated with increasing concentrations of JQ1 (which is a known inhibitor of bromodomain and extraterminal domain (BET) family proteins), inventive bifunctional compounds 1 to 5, and inhibitors I-1 and I-2.

FIG. 4A-FIG. 41 are fluorescence polarization competition assays of mutant BRD2_{BD2 L383}v and TAMRA-JQ1 probe with titration of JQ1, inventive bifunctional compounds 1 to 5, and known inhibitors I-1 and I-2. IC₅₀ values are reported.

FIG. 5A.-FIG. 5I are fluorescence polarization competition assays of mutant BRD4_{BD1} L94V and TAMRA-JQ1 probe with titration of JQ1, inventive bifunctional compounds 1 to 5, and inhibitors I-1 and I-2. IC₅₀ values are reported.

FIG. 6A.-FIG. 61 are fluorescence polarization competition assays of mutant BRD4_{BD2 L387}v and TAMRA-JQ1 probe with titration of JQ1, inventive bifunctional compounds 1 to 5, and inhibitors I-1 and I-2. IC₅₀ values are reported.

FIG. 7A.-FIG. 71 are fluorescence polarization competition assay of mutant BRD4_{BD2} and TAMRA-JQ1 probe with titration of JQ1, inventive bifunctional compounds 1 to 5, and inhibitors I-1 and I-2. IC₅₀ values are reported.

FIG. 8A-FIG. 8B are scatter plots depicting selectivity of inventive bifunctional compounds 1 and 5 in MOLT4 cells that were treated with 1 μM bifunctional compound 1 (FIG. 8A), 1 μM bifunctional compound 5 (FIG. 8B), or DMSO control for 5 h.

FIG. 9A-FIG. 9C are a set of images of structural alignments of bromodomain BRD4_{BD1} L94V (PDB 503C) and BRD4_{BD1} in complex with known compound dBET6 (FIG. 9A), dBET57 (FIG. 9B), or ZXH-3-26 (FIG. 9C), and DDB1-CRBN. FIG. 9A shows a structural alignment of bromodomain BRD4_{BD1} L94V (PDB 503C) and BRD4_{BD1} in complex with dBET6 and DDB1-CRBN (PDB 6BOY). BRD4_{BD1}L94V ‘hole’ residue is highlighted in red. FIG. 9B shows a structural alignment of bromodomain BRD4_{BD1}L94V (PDB 503C) and BRD4_{BD1} in complex with dBET57 and DDB1-CRBN (PDB 6BNB). BRD4_{BD1}L94V ‘hole’ residue is highlighted in red and CRBN Y355 in cyan. Thalidomide is modelled in by aligning CRBN C-terminal domain in crystal structure of DDB1-CRBN-thalidomide (PDB 4CI1) to C-terminal domain of CRBN in dBET57 complex (PDB 6BNB). FIG. 9C shows a structural alignment of bromodomain BRD4_{BD1}L94V (PDB 503C) and Rosetta docking model (Nowak et al., Nat Chem Biol 14 (7):706-714 (2018)) of BRD4_{BD1} complex with ZXH-3-26 and DDB1-CRBN. BRD4_{BD1} L94V ‘hole’ residue is highlighted in red. Lenalidomide is modelled in by aligning CRBN (PDB 5FQD) to CRBN in ZXH-3-26 model.

FIG. 10A-FIG. 10B are a set of plots of fluorescence polarization competition assays of BRD4_{BD1} and mutant BRD4_{BD1}. FIG. 10A shows dose response displacement of fluorescein isothiocyanate (FITC)-JQ1 probe by JQ1, inventive bifunctional compounds 1 to 5, and inhibitors I-1 and I-2 in BRD4_{BD1} and BRD4_{BD1} L94V assays. Data is shown as means±st. dev. of N=3 independent replicates. FIG. 10B shows a comparison of dose responses between BRD4_{BD1} and BRD4_{BD1} L94V for inventive bifunctional compounds 1 and 5. Data is shown as means±st. dev. of N=3 independent replicates.

FIG. 11A-FIG. 11E are a set of plots showing degradation selectivity profile for inventive bifunctional compounds 1 and 5. FIG. 11A-FIG. 11C show quantitative assessment of cellular degradation using BRD4_{BD1}-enhanced green fluorescent protein (EGFP) reporter assay. Cells stably expressing BRD4_{BD1} (FIG. 11A), BRD4_{BD2} (FIG. 11B), or BRD4_{BD1}L94V (FIG. 11C) were treated with increasing concentrations of JQ1, inventive bifunctional compounds 1 to 5, and inhibitors I-1 and I-2 for 5 h and the EGFP to mCherry ratio relative to DMSO was quantified using laser scanning cytometer (Acumen®). FIG. 11D-FIG. 11E show cellular degradation selectivity between BRD4_{BD1}L94V mutant and BRD4_{BD1} wild type reporter with inventive bifunctional compounds 1 to 5. Data as in FIG. 11A-FIG. 11C. Data for independent replicates are presented as means±st. dev. of N=3 replicates for BRD4_{BD1}L94V and BRD4_{BD1}, and N=2 for BRD4_{BD2}.

FIG. 12A-FIG. 12G are set of a diagram and plots of Acumen® and Nano-Glo® HiBit lytic assays showing proof-of-principle simultaneous use of dTAG and BRD4_{BD1}L94V degron systems. FIG. 12A shows a schematic of the degradation system. Flp293T cells stably expressing BRD4_{BD1}L94V-EGFP with mCherry reporter were engineered to stably express HiBit-FKBP12-F36V-KRAS G12C fusion. The use of EGFP/mCherry system as well as orthogonal Nano-Glo® HiBit lytic detection system allowed for sequential read of the protein abundance by Acumen® followed by Nano-Glo® HiBit lytic assay (Promega™). FIG. 12B is a plot of Acumen® EGFP/mCherry assay showing degradation of BRD4_{BD1}L94V-EGFP by CRBN based bifunctional compound 1 at 5 h timepoint in presence or absence of CRBN based known bifunctional compound (degrader) dTAG13. FIG. 12C is a plot of HiBit lytic assay showing degradation of FKBP12 F36V-KRAS G12C by dTAG13 in presence or absence of bifunctional compound 1. The readout was performed on the same assay plate at 7 h timepoint. FIG. 12D is a plot of Acumen® EGFP/mCherry assay showing degradation of BRD4_{BD1}L94V-EGFP. Dose response of dTAG^{v}-1 in the presence of 1 or 0.1 μM of bifunctional compound 1. FIG. 12E is a plot of HiBit lytic assay showing degradation of FKBP12 F36V-KRAS G12C by dTAG^{v}-1 in presence or absence of bifunctional compound 1. The readout was performed on the same assay plate at 7 h timepoint. FIG. 12F is a plot showing degradation of BRD4_{BD1}L94V-EGFP by bifunctional compound 1 in presence of dTAG^{v}-1. FIG. 12G is a plot showing degradation of BRD4_{BD1}L94V-EGFP by bifunctional compound 1 in presence of P-gp inhibitor Tariquidar. Data in FIG. 12D-FIG. 12G are presented as means±st. dev. of N=3 replicates. All Acumen® data were analyzed using Cellista™ software (TTP Labtech).

FIG. 13A-FIG. 13E are a set of plots of fluorescence polarization assays showing binding of JQ1-FITC probe to BRD4_{BD1} (FIG. 13A), BRD4_{BD2} (FIG. 13B) and mutants BRD4_{BD1}L94V (FIG. 13C), BRD4_{BD2}L387V (FIG. 13D), and BRD2_{BD2}L383V (FIG. 13E). Wild type and mutant proteins were titrated to 10 nM of JQ1-FITC fluorescent probe, or 2.5 nM probe for BRD4_{BD1}. Data for N=3 independent replicates are presented as means±st. dev. The apparent K_{d} values were estimated by fitting in “one site-total” binding model in GraphPad Prism v8.

FIG. 14A-FIG. 14E are a set of plots of fluorescence polarization assays showing competitive titration of compound JQ1, inventive bifunctional compounds 1 to 5, and inhibitors I-1 and I-2 to the bromodomains BRD4_{BD1} (FIG. 14A), BRD4_{BD2} (FIG. 14B) and mutants BRD4_{BD1}L94V (FIG. 14C), BRD4_{BD2}L387V (FIG. 14D), and BRD2_{BD2}L383V (FIG. 14E) containing JQ1-FITC probe at 10 nM (2.5 nM for BRD4_{BD1}). Data for N=3 independent replicates are presented as means±st. dev.

FIG. 15A-FIG. 15H are a set of plots showing simultaneous degradation of BRD4_{BD1}L94V-EGFP and HiBit-dTAG-KRAS G12C stably expressed in Flp293T cells. FIG. 15A is a plot of quantitative assessment of degradation of BRD4_{BD1}L94V-GFP by inventive bifunctional compound 1 in presence of dTAG^{v}-1 at 5 h using Acumen® (TTP Labtech). FIG. 15B is a plot of HiBit assay of the plate in FIG. A measuring degradation of HiBit-dTAG-KRAS G12C at 7 h timepoint. FIG. 15C is a plot of quantitative assessment of degradation of BRD4_{BD1}L94V-GFP by inventive bifunctional compound 5 in presence of dTAG^{v}-1 at 5 h using Acumen® (TTP Labtech). FIG. 15D is a plot of HiBit assay of the plate in FIG. 15C measuring degradation of HiBit-dTAG-KRAS G12C at 7 h timepoint. FIG. 15E is a plot of quantitative assessment of degradation of BRD4_{BD1}L94V-GFP by inventive bifunctional compound 1 in presence of dTAG13 at 5 h using Acumen® (TTP Labtech). FIG. 15F is a plot of HiBit assay of the plate in FIG. 15E measuring degradation of HiBit-dTAG-KRAS G12C at 7 h timepoint. FIG. 15G is a plot of quantitative assessment of degradation of BRD4_{BD1}L94V-GFP by inventive bifunctional compound 5 in presence of dTAG13 at 5 h using Acumen® (TTP Labtech). FIG. 15H is a plot of HiBit assay of the plate in FIG. 15G measuring degradation of HiBit-dTAG-KRAS G12C at 7 h timepoint. All of the data in the figure is a singlicate measurement from the same assay plate, N=1. Acumen® data was analyzed using CellProfiler™.

FIG. 16A-FIG. 16D are a set of plots showing simultaneous degradation of BRD4_{BD1}L94V-EGFP and HiBit-dTAG-KRAS G12C stably expressed in Flp293T cells. FIG. 16A is a plot of Acumen® GFP/mCherry assay showing degradation of BRD4_{BD1}L94V-GFP by CRBN based inventive bifunctional compound 1 at 5 h timepoint in presence or absence of CRBN based degrader dTAG13. FIG. 16B is a plot of HiBit lytic assay showing degradation of FKBP12 F36V-KRAS G12C by dTAG13 in presence or absence of inventive bifunctional compound 1. The readout was performed on the same assay plate as in FIG. 16A at 7 h timepoint. FIG. 16C is a plot of Acumen® GFP/mCherry assay showing degradation of BRD4_{BD1}L94V-GFP by CRBN based inventive bifunctional compound 1 at 5 h timepoint in presence or absence of CRBN based degrader dTAG^{v}-1. FIG. 16D is a plot of HiBit lytic assay showing degradation of FKBP12 F36V-KRAS G12C by dTAG^{v}-1 in presence or absence of inventive bifunctional compound 1. The readout was performed on the same assay plate as in FIG. 16C at 7 h timepoint. All of the data in the figure is a singlicate measurement from the same assay plate, N=1. Acumen® data was analyzed using CellProfiler™.

FIG. 17A-FIG. 17B are a set of plots showing pharmacokinetics profiles of inventive bifunctional compounds 1 and 5. FIG. 17A is plot showing mean plasma concentration-time profiles of bifunctional compound 5 after single IV or IP administration in male C57BL/6 mice (N=3/time point). FIG. 17B is plot Mean plasma concentration-time profiles of bifunctional compound 1 after single IV or IP administration in male C57BL/6 mice (N=3/time point). The corresponding BRD4_{BD1}L94V reporter line DC_{50,6h} is represented with a dashed red line.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “haloalkyl” refers to an alkyl group as defined herein that is substituted with one or more (e.g., 1, 2, 3, or 4) halo groups.

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” includes two hydrocarbyl groups covalently linked by an oxygen.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2] octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol [4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}— heterocyclyl where R^{c} is an alkylene chain.

The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered to embrace any and all such resonance structures.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

To the extent not disclosed otherwise for any particular group(s), representative examples of substituents may thus include alkyl, substituted alkyl (e.g., C₁-C₆, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g, C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the bifunctional compound and the targeted protein, which in this invention is a mutant protein bromodomain and extraterminal domain (BET) family protein (e.g., bromodomain-containing protein 2 (BRD2), BRD3, BRD4, and bromodomain testis-specific protein (BRDT))-tagged protein), typically refers to an inter-molecular interaction that is substantially specific or selective in that binding of the targeting ligand with other proteins present in the cell is functionally insignificant.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient or selective to achieve recruitment of the ligase to the targeted degradation and the selective degradation of the targeted protein.

Broadly, the present invention is directed to a bifunctional compound having a structure represented by formula I:

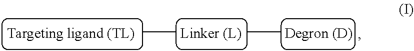

wherein the targeting ligand is represented by any one of structures TL1 to TL3:

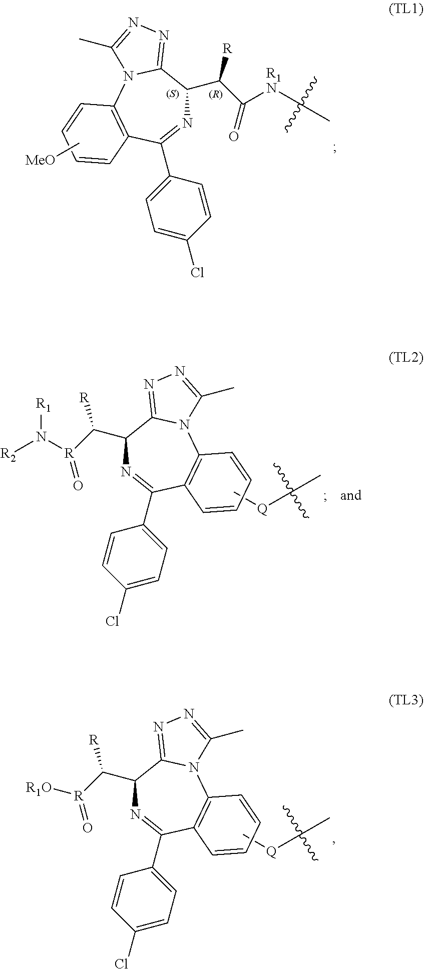

wherein
R represents methyl, ethyl, propyl, isopropyl, or allyl,
R₁ and R₂ independently represent H, methyl, or ethyl, and
Q represent a bond, CH₂, N, or O;
the linker represents a moiety that connects covalently the degron and the targeting ligand; and the degron represents a ligand that binds an E3 ubiquitin ligase, or a pharmaceutically acceptable salt or stereoisomer thereof.

Bifunctional compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present invention may be made and used in the form of individual isomers and substantially free of other isomers, or in the form of a mixture of various isomers, e.g., racemic mixtures of stereoisomers.

In some embodiments, the bifunctional compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, bifunctional compounds of formula (I) embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Linkers

The linker (“L”) provides a covalent attachment between the targeting ligand and the degron. The structure of linker may not be critical, provided it is substantially non-interfering with the activity of the targeting ligand or the degron. In some embodiments, the linker includes an alkylene chain (e.g., having 2-20 alkylene units). In other embodiments, the linker may include an alkylene chain or a bivalent alkylene chain, either of which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker may include a C₁-C₁₂ alkylene chain terminating in NH-group wherein the nitrogen is also bound to the degron.

In some embodiments, the linker includes an alkylene chain having 1-10 alkylene units that is interrupted by and/or terminating in

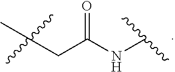 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of alkylene linkers that may be suitable for use in the present invention include the following:

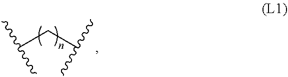

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

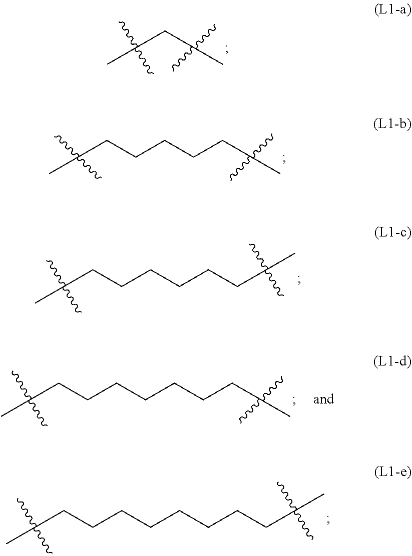

alkylene chains terminating in various functional groups (as described above), examples of which are as follows:

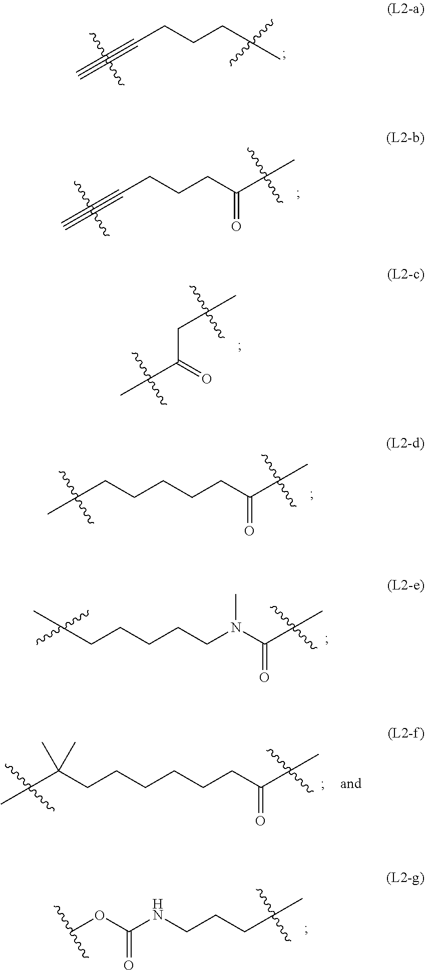

alkylene chains interrupted with various functional groups (as described above), examples of which are as follows:

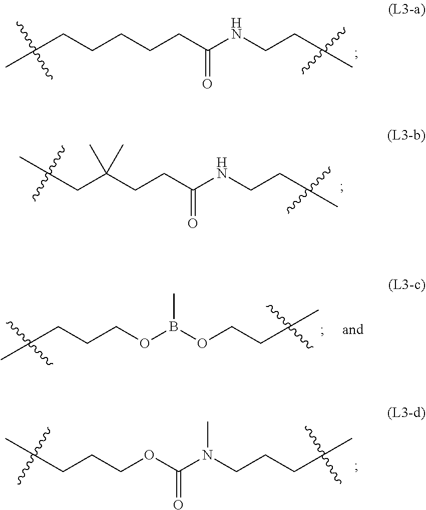 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with heterocyclene groups, e.g.,

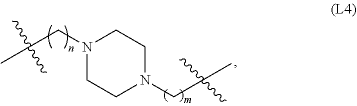

wherein m and n are independently integers of 0-10, examples of which include:

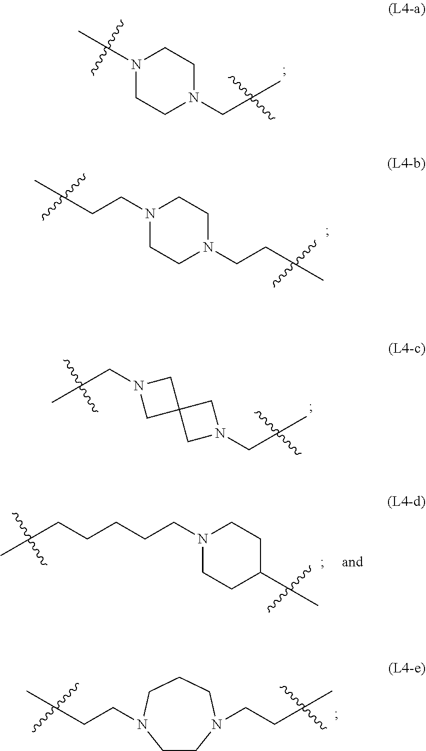 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

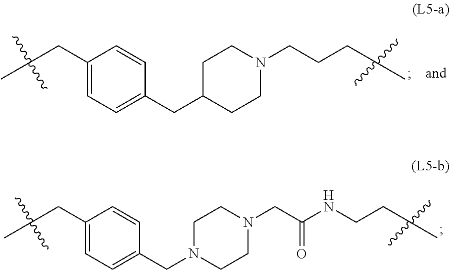 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

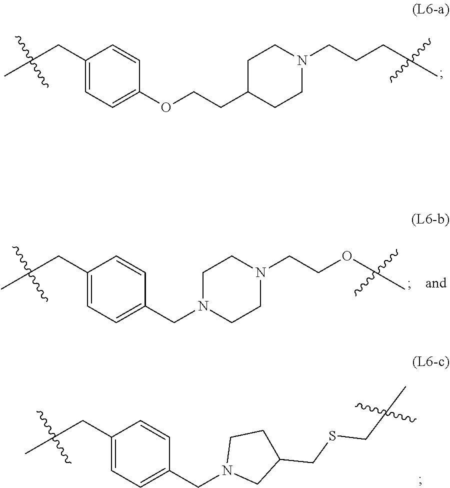 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and

alkylene chains interrupted by a heteroatom such as N, O or B, e.g.,

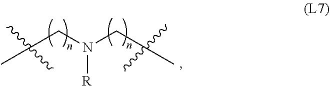

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C1 to C4 alkyl, an example of which is

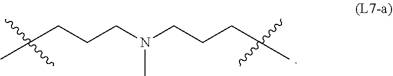 [SMILES: *CCCN(C)CCC*]

In some embodiments, the linker may include a polyethylene glycol chain which may terminate (at either or both termini) in at least one of —S—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 2-8 PEG units and terminating in

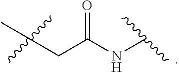 [SMILES: *CC(=O)N*]

Examples of linkers that include a polyethylene glycol chain include:

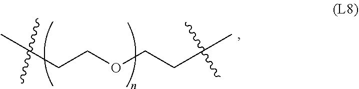

wherein n is an integer of 2-10, examples of which include:

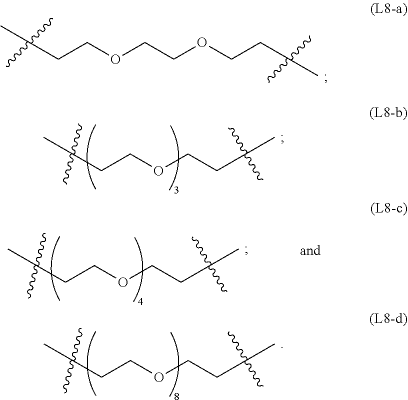

In some embodiments, the polyethylene glycol linker may terminate in a functional group, examples of which are as follows:

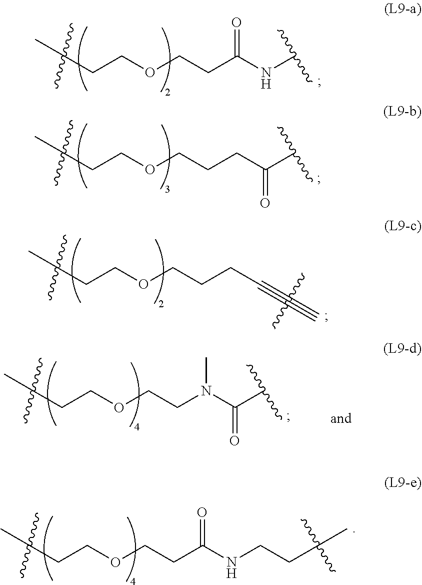

In some embodiments, the bifunctional compound of formula (I) includes a linker that is represented by structure L10:

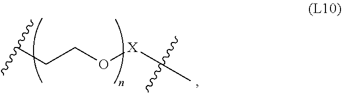

wherein n is an integer of 0-8; and
X is absent or C₁ to C₁₂ alkyl.

In some embodiments, the linker is represented by any one of structures L11 to L15:

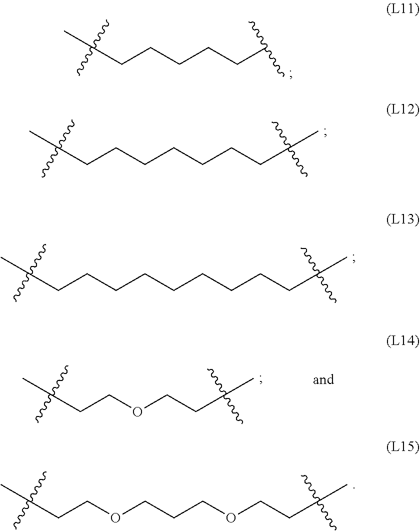 [SMILES: *CCCCC* ; *CCCCCCC* ; *CCCCCCCCC* ; *CCOCC* ; *CCOCCCOCC*]

Thus, in some embodiments, the bifunctional compounds of this invention may be represented by any one of structures I-1 to 1-6:

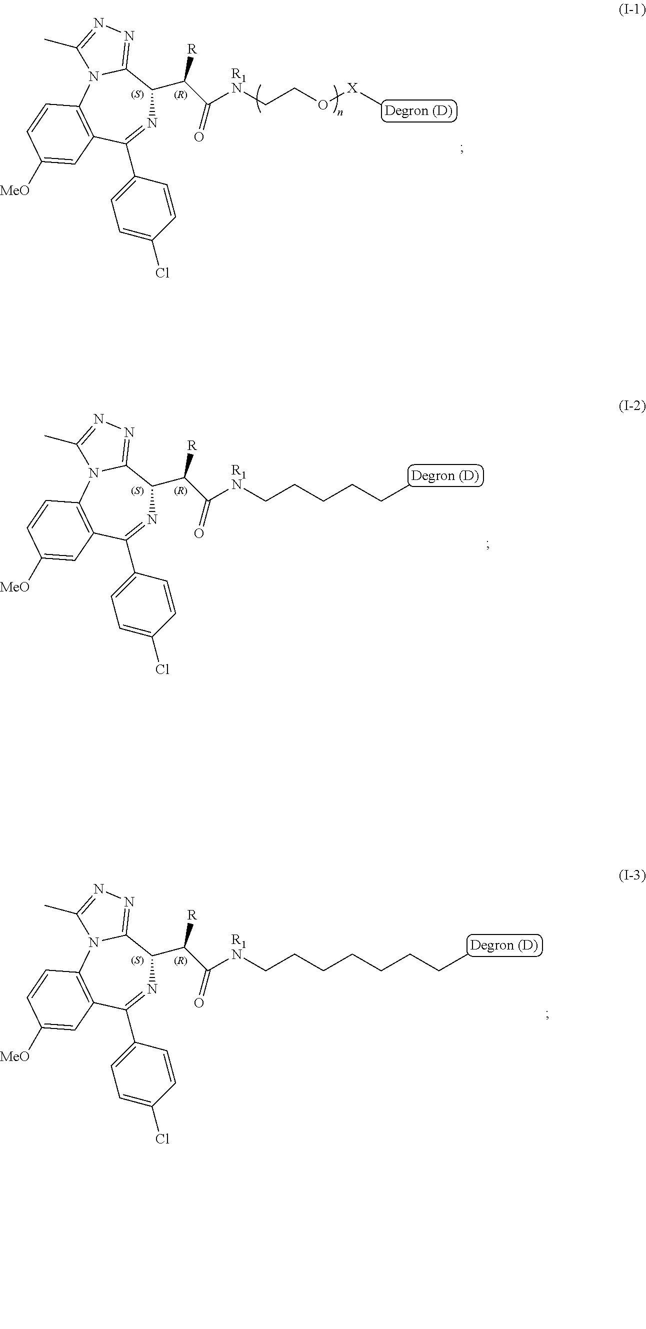

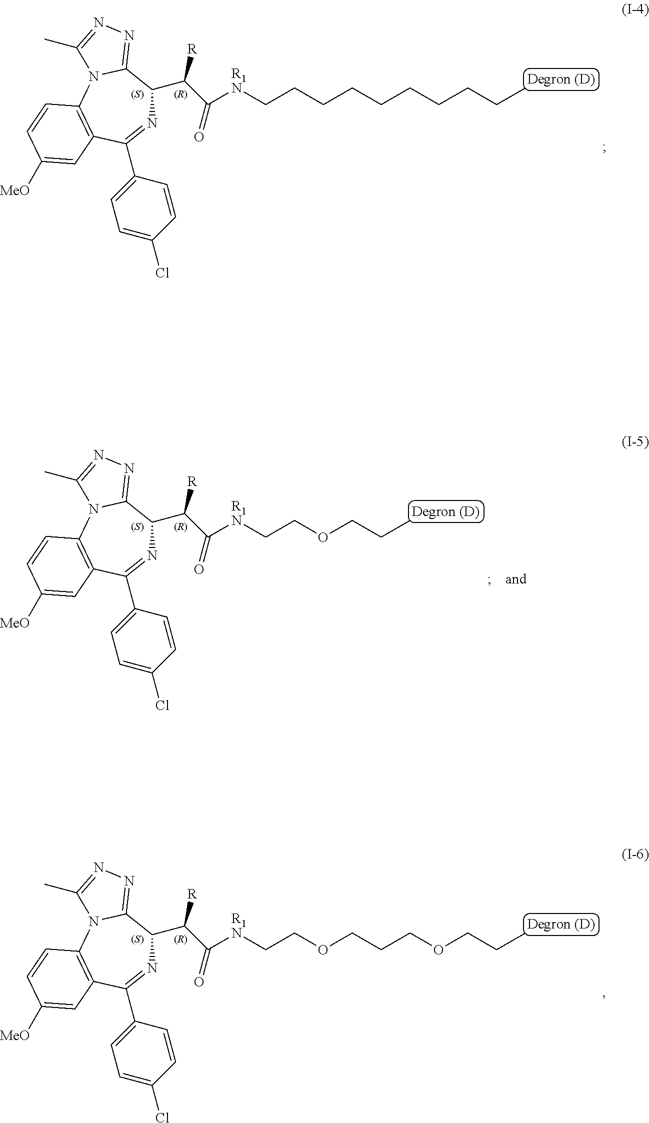

wherein n is an integer from 0-8;
R represents methyl, ethyl, propyl, isopropyl, or allyl;
R₁ represents H, methyl, or ethyl; and
X is absent or C1 to C12 alkylene,
or a pharmaceutically acceptable salt or stereoisomer thereof.

### Degrons

The degron (“D”) is a functional moiety or ligand that binds an E3 ubiquitin ligase.

In some embodiments, the bifunctional compound of formula (I) includes a degron that binds cereblon. Representative examples of degrons that bind cereblon and which may be suitable for use as degrons in the present invention are described in U.S. Patent Application Publication 2018/0015085 (e.g., the indolinones such as isoindolinones and isoindoline-1,3-diones embraced by formulae IA ad IA′ therein, and the bridged cycloalkyl compounds embraced by formulae IB and IB′ therein).

In some embodiments, the bifunctional compounds of formula (I) include a degron that binds cereblon, and is represented by any one of structures D1-a to D1-d:

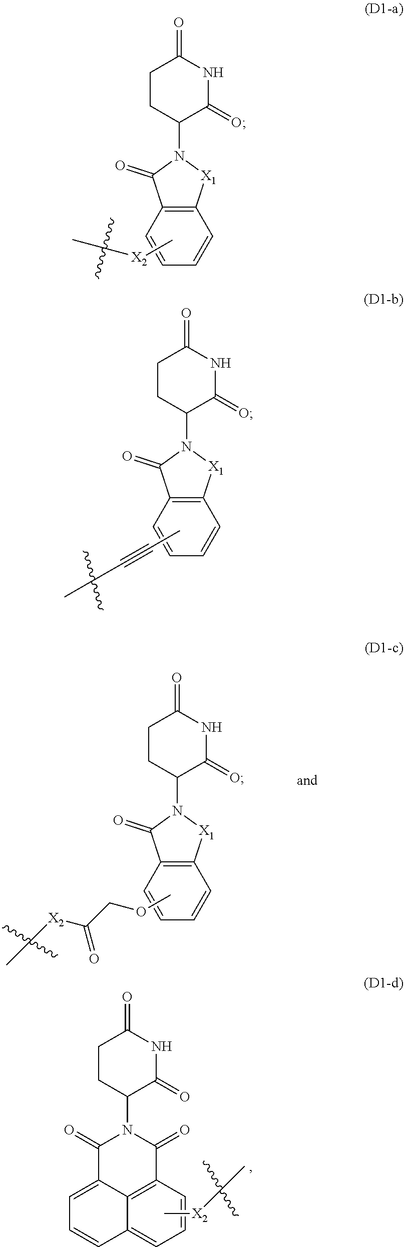

wherein X₁ is CH₂ or C(O) and X₂ is absent, CH₂, NH, or O.

In some embodiments, the degron binds a Von Hippel-Lindau (VHL) tumor suppressor. Representative examples of degrons that bind VHL are as follows:

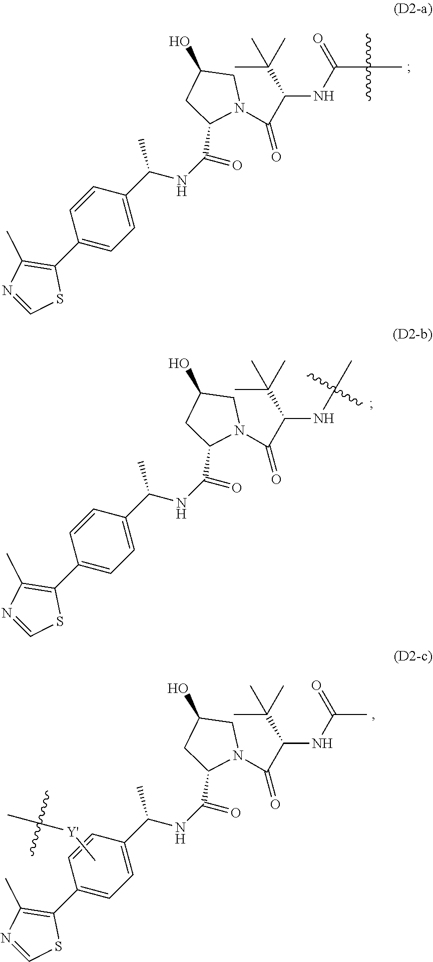

wherein Y′ is a bond, N, O or C;

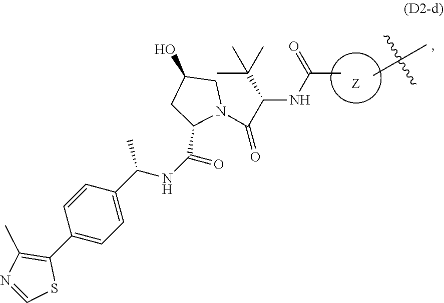

wherein Z is a C₅-C₆ carbocyclic or a

C₅-C₆ heterocyclic group;

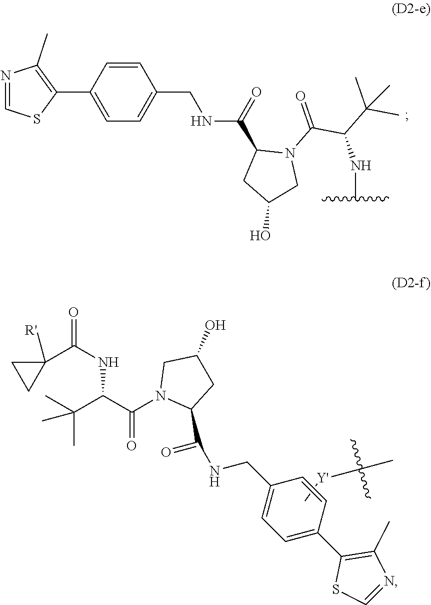

wherein F or CN, and Y′ is a bond, N, O or C; and

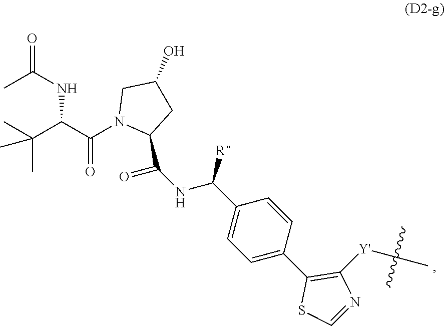

wherein R″ is H or methyl, and Y′ is a bond, N, O or C.

In some embodiments, Z is

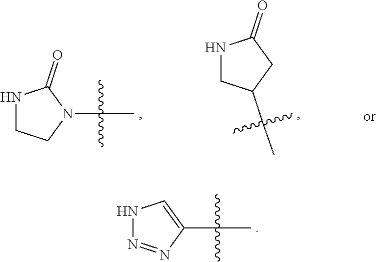 [SMILES: *N1CCNC1=O ; *C1CNC(=O)C1 ; *c1c[nH]nn1]

Yet other degrons that bind VHL and which may be suitable for use as degrons in the present invention are disclosed in U.S. Patent Application Publication 2017/0121321 A1.

Thus, in some embodiments, the bifunctional compounds of the present invention are represented by any structures generated by the combination of structures (I-1) to (I-6), L1-L15 and the structures of the degrons described herein (e.g., (D1a) to (D1d) and (D2-a) to (D2-0), or a pharmaceutically acceptable salt or stereoisomer thereof.

In some embodiments, the bifunctional compounds of the present invention are represented by any one of structures 1 to 7:

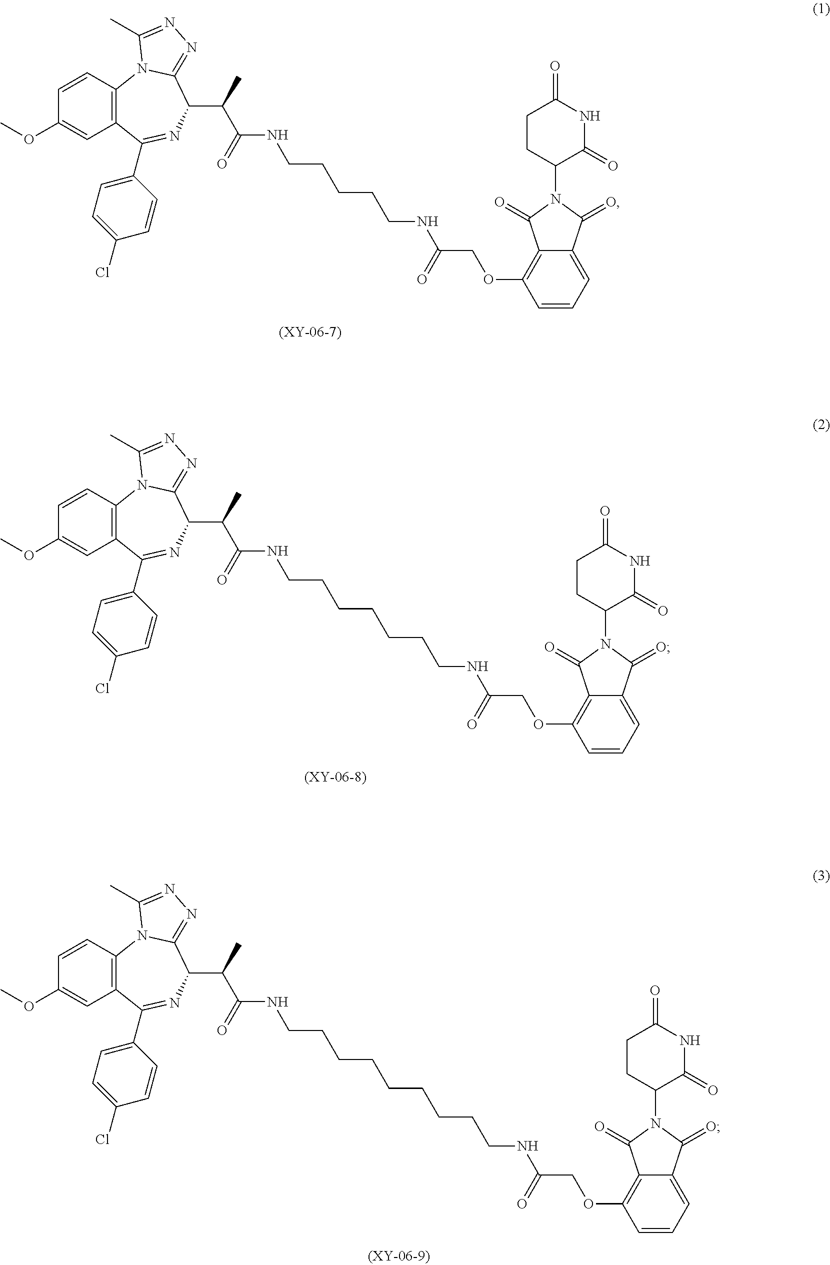 [SMILES: COc1ccc2c(c1)C(c1ccc(Cl)cc1)=N[C@@H]([C@@H](C)C(=O)NCCCCCNC(=O)COc1cccc3c1C(=O)N(C1CCC(=O)NC1=O)C3=O)c1nnc(C)n1-2 ; COc1ccc2c(c1)C(c1ccc(Cl)cc1)=N[C@@H]([C@@H](C)C(=O)NCCCCCCCNC(=O)COc1cccc3c1C(=O)N(C1CCC(=O)NC1=O)C3=O)c1nnc(C)n1-2 ; COc1ccc2c(c1)C(c1ccc(Cl)cc1)=N[C@@H]([C@@H](C)C(=O)NCCCCCCCCCNC(=O)COc1cccc3c1C(=O)N(C1CCC(=O)NC1=O)C3=O)c1nnc(C)n1-2]

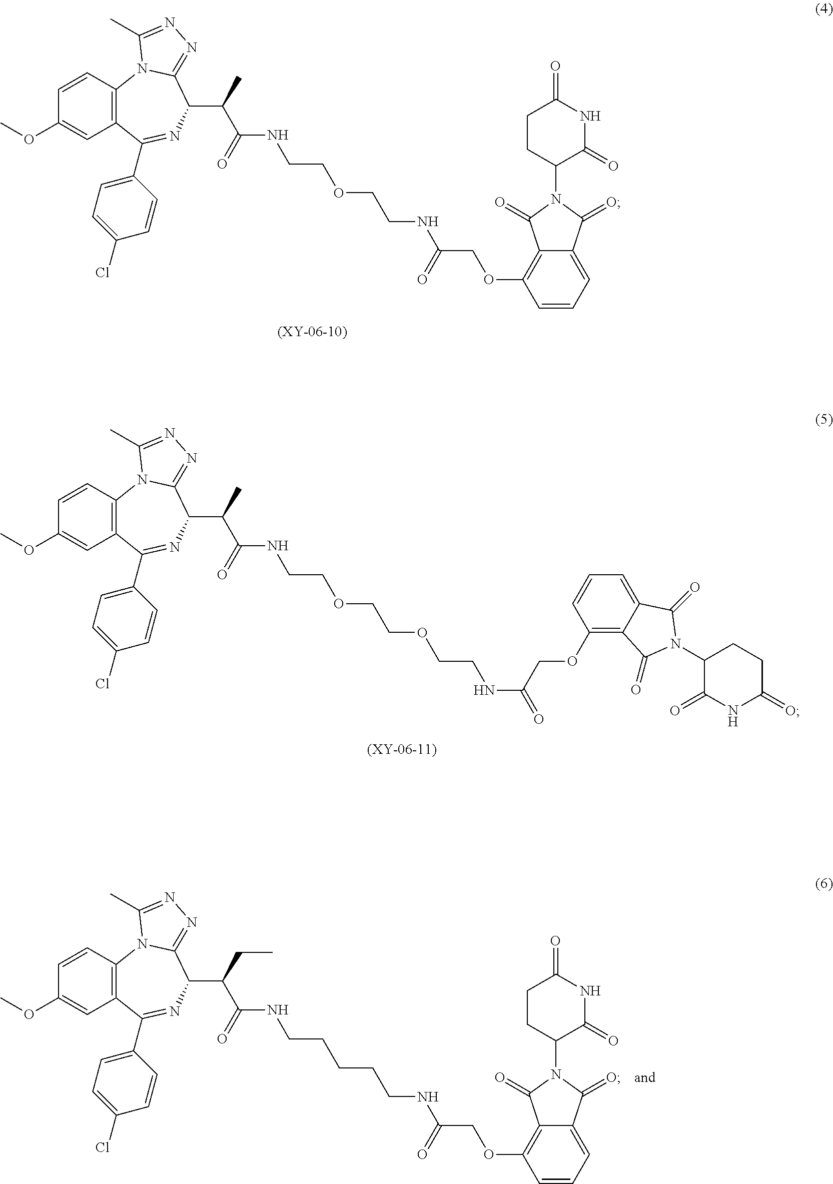 [SMILES: COc1ccc2c(c1)C(c1ccc(Cl)cc1)=N[C@@H]([C@@H](C)C(=O)NCCOCCNC(=O)COc1cccc3c1C(=O)N(C1CCC(=O)NC1=O)C3=O)c1nnc(C)n1-2 ; COc1ccc2c(c1)C(c1ccc(Cl)cc1)=N[C@@H]([C@@H](C)C(=O)NCCOCCOCCNC(=O)COc1cccc3c1C(=O)N(C1CCC(=O)NC1=O)C3=O)c1nnc(C)n1-2 ; CC[C@@H](C(=O)NCCCCCNC(=O)COc1cccc2c1C(=O)N(C1CCC(=O)NC1=O)C2=O)[C@@H]1N=C(c2ccc(Cl)cc2)c2cc(OC)ccc2-n2c(C)nnc21]

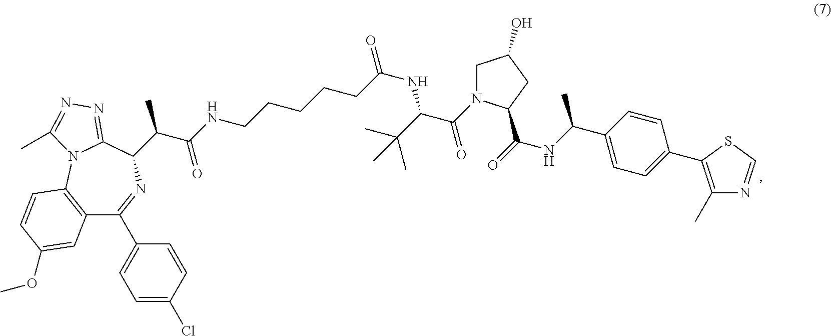 [SMILES: COc1ccc2c(c1)C(c1ccc(Cl)cc1)=N[C@@H]([C@@H](C)C(=O)NCCCCCC(=O)N[C@H](C(=O)N1C[C@H](O)C[C@H]1C(=O)N[C@@H](C)c1ccc(-c3scnc3C)cc1)C(C)(C)C)c1nnc(C)n1-2]

and pharmaceutically acceptable salts and stereoisomers thereof.

### Methods of Synthesis

Broadly, the inventive bifunctional compounds or pharmaceutically acceptable salts or stereoisomers thereof, may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

The Bifunctional Compound Targeting Protein (dTAG)

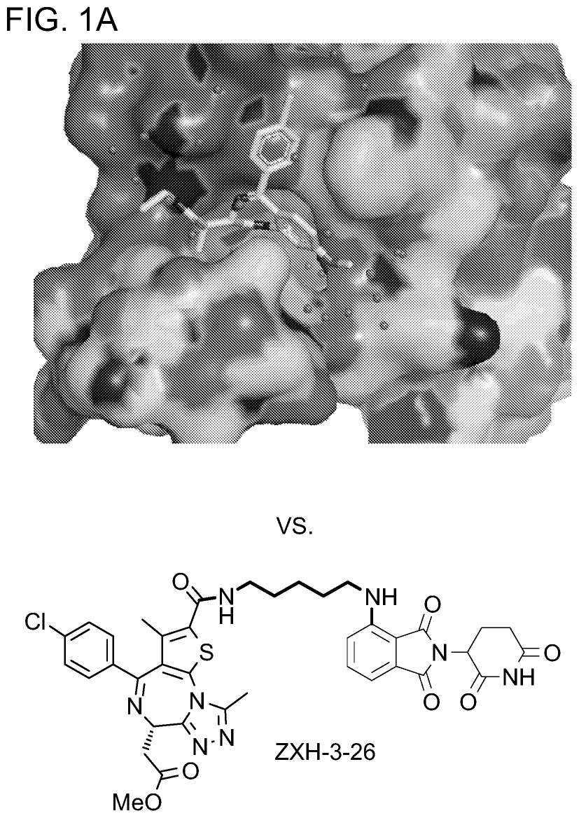

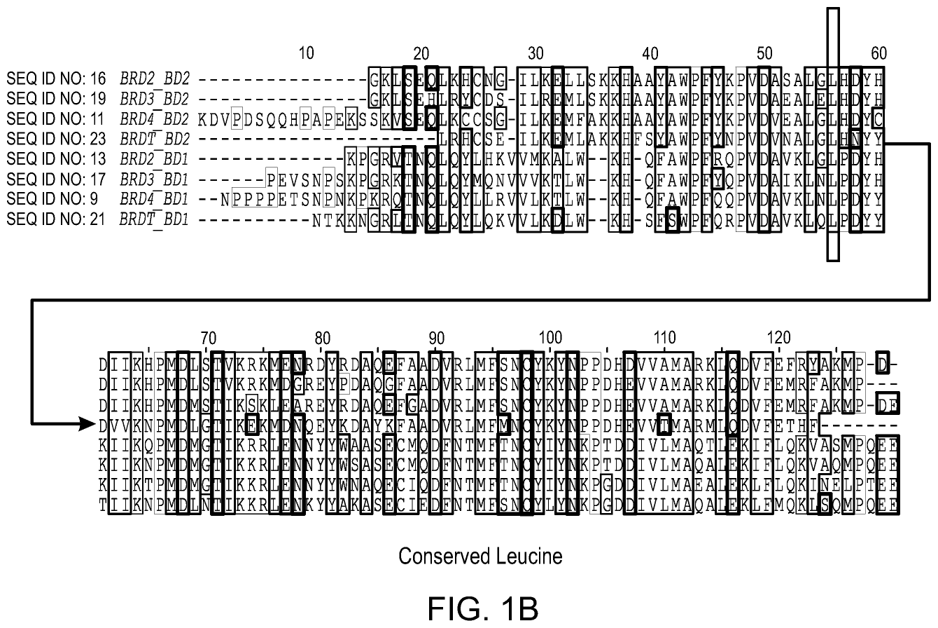

Another aspect of the present invention is directed to a degradation tag (dTAG) that is a modified or mutant BET family protein (e.g., BRD2, BRD3, BRD4, and BRDT) that binds an inventive bifunctional compound or a nucleic acid encoding the dTAG. Broadly, the dTAG is a non-naturally occurring (e.g., modified or mutant) BET family protein, or a bromodomain-containing fragment thereof (e.g., BRD4_{BD1}, BRD4_{BD2}, BRD2_{BD1}, BRD2_{BD2}, BRD3_{BD1}, BRD3_{BD2}, BRDT_{BD1}, and BRDT_{BD2}) which includes a BET-binding pocket that contains an amino acid substitution of a conserved Leu residue in the BET-binding pocket. The conserved Leu substitution serves to enlarge the binding pocket for BET ligands (FIG. 1A). A sequence alignment of BRD2/3/4/T showing the conserved Leu residues is illustrated in FIG. 1B.

By way of example, the amino acid and nucleotide sequences of native (wild-type) human BRD4, BRD3, BRD2 and BRDT are as follows:

An exemplary human BRD4 amino acid sequence, NCBI Accession No. NP 490597, version NP_490597.1, incorporated herein by reference, is set forth below:

| (SEQ ID NO: 1) | |
|---|---|
| 1 | msaesgpgtr lrnlpvmgdg letsqmsttq aqaqpqpana astnppppet snpnkpkrqt |
| 61 | nqlqyllrvv lktlwkhqfa wpfqqpvdav klnlpdyyki iktpmdmgti kkrlennyyw |
| 121 | naqeciqdfn tmftncyiyn kpgddivlma ealeklflqk inelpteete imivqakgrg |
| 181 | rgrketgtak pgvstvpntt qastppqtqt pqpnpppvqa tphpfpavtp dlivqtpvmt |
| 241 | vvppqplqtp ppvppqpqpp papapqpvqs hppiiaatpq pvktkkgvkr kadtttptti |
| 301 | dpiheppslp pepkttklgq rressrpvkp pkkdvpdsqq hpapeksskv seqlkccsgi |
| 361 | lkemfakkha ayawpfykpv dvealglhdy cdiikhpmdm stiksklear eyrdaqefga |
| 421 | dvrlmfsncy kynppdhevv amarklqdvf emrfakmpde peepvvavss pavppptkvv |
| 481 | appsssdsss dsssdsdsst ddseeeraqr laelqeqlka vheqlaalsq pqqnkpkkke |
| 541 | kdkkekkkek hkrkeeveen kkskakeppp kktkknnssn snvskkepap mkskppptye |
| 601 | seeedkckpm syeekrqlsl dinklpgekl grvvhiiqsr epslknsnpd eieidfetkk |
| 661 | pstlrelery vtsclrkkrk pqaekvdvia gsskmkgfss sesesssess ssdsedsete |
| 721 | mapkskkkgh pgreqkkhhh hhhqqmqqap apvpqqpppp pqqppppppp qqqqqppppp |
| 781 | pppsmpqqaa pamksspppf iatqvpvlep qlpgsvfdpi ghftqpilhl pqpelpphlp |
| 841 | qppehstpph lnqhavvspp alhnalpqqp srpsnraaal ppkparppav spaltqtpll |
| 901 | pqppmaqppq vlledeeppa ppltsmqmql ylqqlqkvqp ptpllpsvkv qsqpppplpp |
| 961 | pphpsvqqql qqqppppppp qpqpppqqqh qppprpvhlq pmqfsthiqq ppppqgqqpp |
| 1021 | hpppgqqppp pqpakpqqvi qhhhsprhhk sdpystghlr eapsplmihs pqmsqfqslt |
| 1081 | hqsppqqnvq pkkqelraas vvqpqplvvv keekihspii rsepfspslr peppkhpesi |
| 1141 | kapvhlpqrp emkpvdvgrp virppeqnap ppgapdkdkq kqepktpvap kkdlkiknmg |
| 1201 | swaslvqkhp ttpsstakss sdsfeqfrra arekeereka lkaqaehaek ekerlrqerm |
| 1261 | rsrededale qarraheear rrqeqqqqqr qeqqqqqqqq aaavaaaatp qaqssqpqsm |
| 1321 | ldqqrelark reqerrrrea maatidmnfq sdllsifeen if |

BRD4 bromodomain 1 (BRD4_{BD1}) and BRD4 bromodomain 2 (BRD4_{BD2}) are highlighted in bold, wherein L94 in BRD4_{BD1} and L387 in BRD4_{BD2} are underlined.

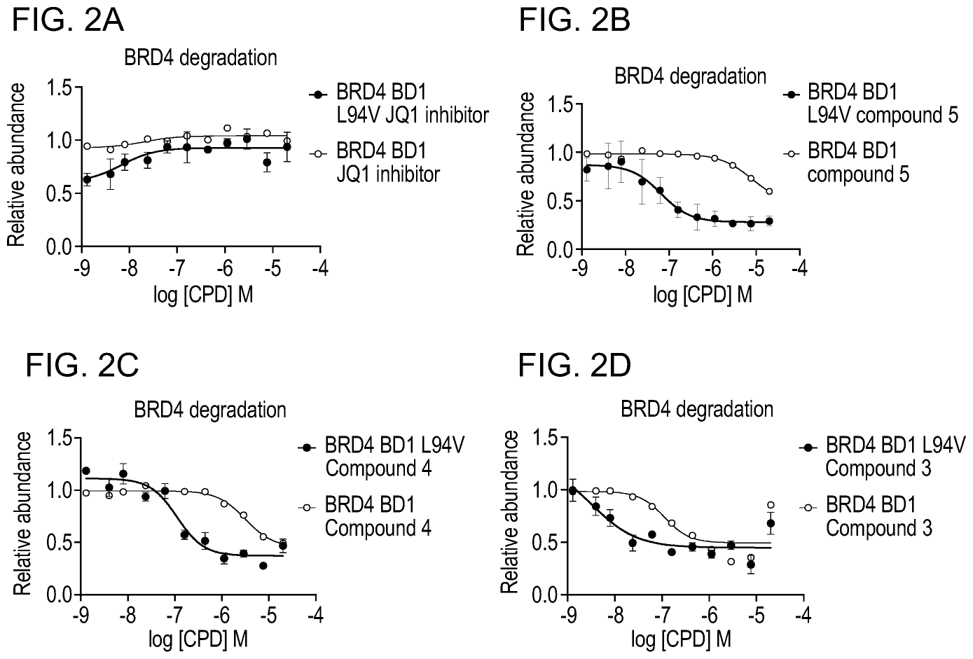

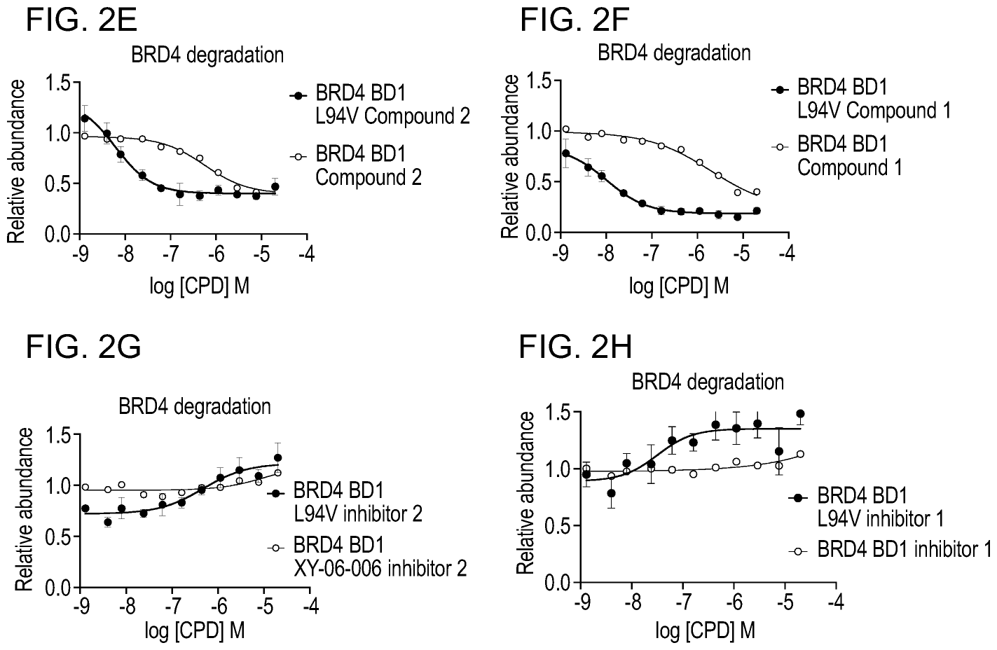

The L94V mutation of BRD4_{BD1} (BRD4_{BD1 L94V}) was used in FIG. 2 with a —C terminal GFP fusion.

An exemplary human BRD4 nucleic acid sequence, NCBI Accession No. NM_058243, version NM_058243.2, incorporated herein by reference, and which encodes SEQ ID NO:1, is set forth below:

| (SEQ ID NO: 2) | |
|---|---|
| 1 | attctttgga atactactgc tagaagtctg acttaagacc cagcttatgg gccacatggc |
| 61 | acccagctgc ttctgcagag aaggcaggcc actgatgggt acagcaaagt gtggtgctgc |
| 121 | tggccaagcc aaagacccgt gtaggatgac tgggcctctg ccccttgtgg gtgttgccac |
| 181 | tgtgcttgag tgcctggtga agaatgtgat gggatcacta gcatgtctgc ggagagcggc |
| 241 | cctgggacga gattgagaaa tctgccagta atgggggatg gactagaaac ttcccaaatg |
| 301 | tctacaacac aggcccaggc ccaaccccag ccagccaacg cagccagcac caaccccccg |
| 361 | cccccagaga cctccaaccc taacaagccc aagaggcaga ccaaccaact gcaatacctg |
| 421 | ctcagagtgg tgctcaagac actatggaaa caccagtttg catggccttt ccagcagcct |
| 481 | gtggatgccg tcaagctgaa cctccctgat tactataaga tcattaaaac gcctatggat |
| 541 | atgggaacaa taaagaagcg cttggaaaac aactattact ggaatgctca ggaatgtatc |
| 601 | caggacttca acactatgtt tacaaattgt tacatctaca acaagcctgg agatgacata |
| 661 | gtcttaatgg cagaagctct ggaaaagctc ttcttgcaaa aaataaatga gctacccaca |
| 721 | gaagaaaccg agatcatgat agtccaggca aaaggaagag gacgtgggag gaaagaaaca |
| 781 | gggacagcaa aacctggcgt ttccacggta ccaaacacaa ctcaagcatc gactcctccg |
| 841 | cagacccaga cccctcagcc gaatcctcct cctgtgcagg ccacgcctca ccccttccct |
| 901 | gccgtcaccc cggacctcat cgtccagacc cctgtcatga cagtggtgcc tccccagcca |
| 961 | ctgcagacgc ccccgccagt gcccccccag ccacaacccc cacccgctcc agctccccag |
| 1021 | cccgtacaga gccacccacc catcatcgcg gccaccccac agcctgtgaa gacaaagaag |
| 1081 | ggagtgaaga ggaaagcaga caccaccacc cccaccacca ttgaccccat tcacgagcca |
| 1141 | ccctcgctgc ccccggagcc caagaccacc aagctgggcc agcggcggga gagcagccgg |
| 1201 | cctgtgaaac ctccaaagaa ggacgtgccc gactctcagc agcacccagc accagagaag |
| 1261 | agcagcaagg tctcggagca gctcaagtgc tgcagcggca tcctcaagga gatgtttgcc |
| 1321 | aagaagcacg ccgcctacgc ctggcccttc tacaagcctg tggacgtgga ggcactgggc |
| 1381 | ctacacgact actgtgacat catcaagcac cccatggaca tgagcacaat caagtctaaa |
| 1441 | ctggaggccc gtgagtaccg tgatgctcag gagtttggtg ctgacgtccg attgatgttc |
| 1501 | tccaactgct ataagtacaa ccctcctgac catgaggtgg tggccatggc ccgcaagctc |
| 1561 | caggatgtgt tcgaaatgcg ctttgccaag atgccggacg agcctgagga gccagtggtg |
| 1621 | gccgtgtcct ccccggcagt gccccctccc accaaggttg tggccccgcc ctcatccagc |
| 1681 | gacagcagca gcgatagctc ctcggacagt gacagttcga ctgatgactc tgaggaggag |
| 1741 | cgagcccagc ggctggctga gctccaggag cagctcaaag ccgtgcacga gcagcttgca |
| 1801 | gccctctctc agccccagca gaacaaacca aagaaaaagg agaaagacaa gaaggaaaag |
| 1861 | aaaaaagaaa agcacaaaag gaaagaggaa gtggaagaga ataaaaaaag caaagccaag |
| 1921 | gaacctcctc ctaaaaagac gaagaaaaat aatagcagca acagcaatgt gagcaagaag |
| 1981 | gagccagcgc ccatgaagag caagccccct cccacgtatg agtcggagga agaggacaag |
| 2041 | tgcaagccta tgtcctatga ggagaagcgg cagctcagct tggacatcaa caagctcccc |
| 2101 | ggcgagaagc tgggccgcgt ggtgcacatc atccagtcac gggagccctc cctgaagaat |
| 2161 | tccaaccccg acgagattga aatcgacttt gagaccctga agccgtccac actgcgtgag |
| 2221 | ctggagcgct atgtcacctc ctgtttgcgg aagaaaagga aacctcaagc tgagaaagtt |
| 2281 | gatgtgattg ccggctcctc caagatgaag ggcttctcgt cctcagagtc ggagagctcc |
| 2341 | agtgagtcca gctcctctga cagcgaagac tccgaaacag agatggctcc gaagtcaaaa |
| 2401 | aagaaggggc accccgggag ggagcagaag aagcaccatc atcaccacca tcagcagatg |
| 2461 | cagcaggccc cggctcctgt gccccagcag ccgcccccgc ctccccagca gcccccaccg |
| 2521 | cctccacctc cgcagcagca acagcagccg ccacccccgc ctcccccacc ctccatgccg |
| 2581 | cagcaggcag ccccggcgat gaagtcctcg cccccaccct tcattgccac ccaggtgccc |
| 2641 | gtcctggagc cccagctccc aggcagcgtc tttgacccca tcggccactt cacccagccc |
| 2701 | atcctgcacc tgccgcagcc tgagctgccc cctcacctgc cccagccgcc tgagcacagc |
| 2761 | actccacccc atctcaacca gcacgcagtg gtctctcctc cagctttgca caacgcacta |
| 2821 | ccccagcagc catcacggcc cagcaaccga gccgctgccc tgcctcccaa gcccgcccgg |
| 2881 | cccccagccg tgtcaccagc cttgacccaa acacccctgc tcccacagcc ccccatggcc |
| 2941 | caaccccccc aagtgctgct ggaggatgaa gagccacctg ccccacccct cacctccatg |
| 3001 | cagatgcagc tgtacctgca gcagctgcag aaggtgcagc cccctacgcc gctactccct |
| 3061 | tccgtgaagg tgcagtccca gcccccaccc cccctgccgc ccccacccca cccctctgtg |
| 3121 | cagcagcagc tgcagcagca gccgccacca cccccaccac cccagcccca gcctccaccc |
| 3181 | cagcagcagc atcagccccc tccacggccc gtgcacttgc agcccatgca gttttccacc |
| 3241 | cacatccaac agcccccgcc accccagggc cagcagcccc cccatccgcc cccaggccag |
| 3301 | cagccacccc cgccgcagcc tgccaagcct cagcaagtca tccagcacca ccattcaccc |
| 3361 | cggcaccaca agtcggaccc ctactcaacc ggtcacctcc gcgaagcccc ctccccgctt |
| 3421 | atgatacatt ccccccagat gtcacagttc cagagcctga cccaccagtc tccaccccag |
| 3481 | caaaacgtcc agcctaagaa acaggagctg cgtgctgcct ccgtggtcca gccccagccc |
| 3541 | ctcgtggtgg tgaaggagga gaagatccac tcacccatca tccgcagcga gcccttcagc |
| 3601 | ccctcgctgc ggccggagcc ccccaagcac ccggagagca tcaaggcccc cgtccacctg |
| 3661 | ccccagcggc cggaaatgaa gcctgtggat gtcgggaggc ctgtgatccg gcccccagag |
| 3721 | cagaacgcac cgccaccagg ggcccctgac aaggacaaac agaaacagga gccgaagact |
| 3781 | ccagttgcgc ccaaaaagga cctgaaaatc aagaacatgg gctcctgggc cagcctagtg |
| 3841 | cagaagcatc cgaccacccc ctcctccaca gccaagtcat ccagcgacag cttcgagcag |
| 3901 | ttccgccgcg ccgctcggga gaaagaggag cgtgagaagg ccctgaaggc tcaggccgag |
| 3961 | cacgctgaga aggagaagga gcggctgcgg caggagcgca tgaggagccg agaggacgag |
| 4021 | gatgcgctgg agcaggcccg gcgggcccat gaggaggcac gtcggcgcca ggagcagcag |
| 4081 | cagcagcagc gccaggagca acagcagcag cagcaacagc aagcagctgc ggtggctgcc |
| 4141 | gccgccaccc cacaggccca gagctcccag ccccagtcca tgctggacca gcagagggag |
| 4201 | ttggcccgga agcgggagca ggagcgaaga cgccgggaag ccatggcagc taccattgac |
| 4261 | atgaatttcc agagtgatct attgtcaata tttgaagaaa atcttttctg agcgcaccta |
| 4321 | ggtggcttct gactttgatt ttctggcaaa acattgactt tccatagtgt taggggcggt |
| 4381 | ggtggaggtg ggatcagcgg ccaggggatg cctcagggcc tggccctcct gcatgctatg |
| 4441 | cccggggcag gcctgacggg cagctgagga ttgcagagcc tgtctgcctt acggccagtc |
| 4501 | ggacagacgt cccgccaccc accacccctc acaggacgtc cgctcagcac acgccttgtt |
| 4561 | acgagcaagt gccggctgga cccaagccct gcatccccac atgcggggca gaggcccttc |
| 4621 | tctccgccaa atgtctacac agtatacaca ggacatcgtt gctgccgccg tgactggttt |
| 4681 | tctgtcccca agaacgtgac gttcgtgatg tcctgcccgc cgggagtctt tccccacacc |
| 4741 | ccagccatcg ccgcccgctc ccaggaggcc agggcaggcc tgcgtgggct ggaggcgggc |
| 4801 | gaggccggcc caccccctcg ctggcactga ctttgccttg aacagacccc ccgaccctcc |
| 4861 | cccacaagcc tttaattgag agccgctctc tgtaagtgtt tgcttgtgca aaagggaata |
| 4921 | gtgccgtgga ggtgtgtgtg tccatggcat ccggagcgag gcgactgtcc tgcgtgggta |
| 4981 | gccctcggcc ggggagtgag gccaccaacc aaagtcagtt ccttcccacc tgtgtttctg |
| 5041 | tttcgttttt ttttttcttt tttttctata tatatttttt gttgaattct attttatttt |
| 5101 | taattctctc ttctcctcca gacacaatgg cactgcttat ctccgaaatg gtgtgatcgt |
| 5161 | ctcctcattg agcagcggct gccaccgcgc tgtgggta |

An exemplary human BRD3 amino acid sequence, NCBI Accession No. XP_006717354, version XP_006717354.1, incorporated herein by reference, is set forth below:

| (SEQ ID NO: 3) | |
|---|---|
| 1 | mstattvapa gipatpgpvn ppppevsnps kpgrktnqlq ymqnvvvktl wkhqfawpfy |
| 61 | qpvdaiklnl pdyhkiiknp mdmgtikkrl ennyywsase cmqdfntmft ncyiynkptd |
| 121 | divlmaqale kiflqkvaqm pqeevellpp apkgkgrkpa agaqsagtqq vaavssvspa |
| 181 | tpfqsvpptv sqtpviaatp vptitanvts vpvppaaapp ppatpivpvv pptppvvkkk |
| 241 | gvkrkadttt pttsaitasr sesppplsdp kqakvvarre sggrpikppk kdledgevpq |
| 301 | hagkkgklse hlrycdsilr emlskkhaay awpfykpvda ealelhdyhd iikhpmdlst |
| 361 | vkrkmdgrey pdaqgfaadv rlmfsncyky nppdhevvam arklqdvfem rfakmpdepv |
| 421 | eapalpapaa pmvskgaess rsseesssds gssdseeera trlaelqeql kavheqlaal |
| 481 | sqapvnkpkk kkekkekekk kkdkekekek hkvkaeeekk akvappakqa qqkkapakka |
| 541 | nstttagrql kkggkqasas ydseeeeegl pmsydekrql sldinrlpge klgrvvhiiq |
| 601 | srepslrdsn pdeieidfet lkpttlrele ryvksclqkk qrkpfsasgk kqaakskeel |
| 661 | aqekkkelek rlqdvsgqls sskkparkek pgsapsggps rlsssssses gsssssgsss |
| 721 | dssdse |

BRD3 bromodomain 1 (BRD3_{BD1}) and BRD3 bromodomain 2 (BRD3_{BD2}) are highlighted in bold, wherein L70 in BRD3_{BD1} and L345 in BRD3_{BD2} are underlined.

An exemplary human BRD3 nucleic acid sequence, NCBI Accession No. XM_006717291, version XM_006717291.3, incorporated herein by reference, and which encodes SEQ ID NO: 3, is set forth below:

| (SEQ ID NO: 4) | |
|---|---|
| 1 | aggctaatta ggcgcgcggt ttgtttacaa acacgggctc ccggcaggtg cgcgccgccc |
| 61 | cgcccgtgcg cggccggggt tcgagggtgg ctcccgcggg cctcggggtg cccggacggg |
| 121 | ggctgcggtg ctggctgcgt gcccgcttct tccatgccgt cctggggcac cggaaaatcc |
| 181 | gccgccaggc gctgtccccg acacgggctg tcgcctggtt gggcccggaa atgggacgtc |
| 241 | gcgctttctc agggagcgta gaagcagcca gggcctctcc aagccgctgc tgtgacagaa |
| 301 | agtgagtgag ctgccggagg atgtccaccg ccacgacagt cgcccccgcg gggatcccgg |
| 361 | cgaccccggg ccctgtgaac ccaccccccc cggaggtctc caaccccagc aagcccggcc |
| 421 | gcaagaccaa ccagctgcag tacatgcaga atgtggtggt gaagacgctc tggaaacacc |
| 481 | agttcgcctg gcccttctac cagcccgtgg acgcaatcaa attgaacctg ccggattatc |
| 541 | ataaaataat taaaaaccca atggatatgg ggactattaa gaagagacta gaaaataatt |
| 601 | attattggag tgcaagcgaa tgtatgcagg acttcaacac catgtttaca aattgttaca |
| 661 | tttataacaa gcccacagat gacatagtgc taatggccca agctttagag aaaatttttc |
| 721 | tacaaaaagt ggcccagatg ccccaagagg aagttgaatt attaccccct gctccaaagg |
| 781 | gcaaaggtcg gaagccggct gcgggagccc agagcgcagg tacacagcaa gtggcggccg |
| 841 | tgtcctctgt ctccccagcg accccctttc agagcgtgcc ccccaccgtc tcccagacgc |
| 901 | ccgtcatcgc tgccacccct gtaccaacca tcactgcaaa cgtcacgtcg gtcccagtcc |
| 961 | ccccagctgc cgccccacct cctcctgcca cacccatcgt ccccgtggtc cctcctacgc |
| 1021 | cgcctgtcgt caagaaaaag ggcgtgaagc ggaaagcaga cacaaccact cccacgacgt |
| 1081 | cggccatcac tgccagccgg agtgagtcgc ccccgccgtt gtcagacccc aagcaggcca |
| 1141 | aagtggtggc ccggcgggag agtggtggcc gccccatcaa gcctcccaag aaggacctgg |
| 1201 | aggacggcga ggtgccccag cacgcaggca agaagggcaa gctgtcggag cacctacgct |
| 1261 | actgcgacag catcctcagg gagatgctat ccaagaagca cgcggcctac gcctggccct |
| 1321 | tctacaagcc agtggatgcc gaggccctgg agctgcacga ctaccacgac atcatcaagc |
| 1381 | acccgatgga cctcagcacc gtgaaaagga agatggatgg ccgagagtac ccagacgcac |
| 1441 | agggctttgc tgctgatgtc cggctgatgt tctcgaattg ctacaaatac aatcccccag |
| 1501 | accacgaggt tgtggccatg gcccggaagc tccaggacgt gtttgagatg aggtttgcca |
| 1561 | agatgccaga tgagcccgtg gaggcaccgg cgctgcctgc ccccgcggcc cccatggtga |
| 1621 | gcaagggcgc tgagagcagc cgtagcagtg aggagagctc ttcggactca ggcagctcgg |
| 1681 | actcggagga ggagcgggcc accaggctgg cggagctgca ggagcagctg aaggccgtgc |
| 1741 | acgagcagct ggccgccctg tctcaggccc cagtaaacaa accaaagaag aagaaggaga |
| 1801 | agaaggagaa ggagaagaag aagaaggaca aggagaagga gaaggagaag cacaaagtga |
| 1861 | aggccgagga agagaagaag gccaaggtgg ctccgcctgc caagcaggct cagcagaaga |
| 1921 | aggctcctgc caagaaggcc aacagcacga ccacggccgg cagacagctg aagaaaggcg |
| 1981 | gcaagcaggc atctgcctcc tacgactcag aggaagagga ggagggcctg cccatgagct |
| 2041 | acgatgaaaa gcgccagctt agcctggaca tcaaccggct gcccggggag aagctgggcc |
| 2101 | gggtagtgca catcatccaa tctcgggagc cctcgctcag ggactccaac cccgacgaga |
| 2161 | tagaaattga ctttgagact ctgaaaccca ccactttgcg ggaactggag agatatgtca |
| 2221 | agtcttgttt acagaaaaag caaaggaaac cgttctcagc aagcgggaag aaacaggcag |
| 2281 | ccaagtcgaa agaggagcta gctcaggaaa agaagaagga gctggaaaag cgtctgcagg |
| 2341 | atgtcagcgg gcagctgagc agcagcaaga agcccgcccg gaaagagaag cccggctcag |
| 2401 | caccctcagg gggcccgtcc aggctcagca gcagcagctc ctccgagtct gggagcagca |
| 2461 | gctccagcgg gtccagctct gacagcagtg actcagaatg aactggcttc ggacagaaca |
| 2521 | ggacagatgg atgtcgcaca cgccgagact ctgccgtacc cctctgtggt tcatattact |
| 2581 | acttctgttc catggtgtgc aggtctgctt cttaattcag tgttatgata tcttccagtt |
| 2641 | tttgctttca taggtcagag atctatcttg tgtgtgcgtt agacttgatg agaaggtgtg |
| 2701 | aactctgcag aaagtctctt cttcatcact gaattcagtc acttggagat gacaacttca |
| 2761 | aatgctaacc cgatgacccc agaaaaccgt gtgagattcg taccgaagaa ccttgtggaa |
| 2821 | tccctttgct taggcccaac ctggtcgata gctcgagaaa gaattttttc caaggaaatg |
| 2881 | tctcggatat gggtactgta tttgaaagct gttagctttg tcaacacgca ttgtccttgt |
| 2941 | catttgggcc ccgagctctg accctcgtgt ctgacgcggc cacctctttc tggaggggct |
| 3001 | gaggacagat gtgcctgctt gtggagacca ggctgggcct aagcgaaggg tcatcgcagc |
| 3061 | cccagcccgg agcgtggagc ccttgggggg tggtcgggtg ggatgtgcgt tctccgctcg |
| 3121 | tggtgatgtc aggagctcct cggagggaac agagcggctg tgtatgcagc ctgcaggttt |
| 3181 | ccatacactg aagcttttac ctcaacttta aaaaaaaaaa aagaagaaaa gatttaaaaa |
| 3241 | aa |

An exemplary human BRD2 amino acid sequence, NCBI Accession No. NP_001106653, version NP_001106653.1, incorporated herein by reference, is set forth below:

| (SEQ ID NO: 5) | |
|---|---|
| 1 | mlqnvtphnk lpgegnagll glgpeaaapg krirkpslly egfesptmas vpalqltpan |
| 61 | ppppevsnpk kpgrvtnqlq ylhkvvmkal wkhqfawpfr qpvdavklgl pdyhkiikqp |
| 121 | mdmgtikrrl ennyywaase cmqdfntmft ncyiynkptd divlmaqtle kiflqkvasm |
| 181 | pqeeqelvvt ipknshkkga klaalqgsvt sahqvpavss vshtalytpp peipttvlni |
| 241 | phpsvisspl lkslhsagpp llavtaappa qplakkkgvk rkadtttptp tailapgspa |
| 301 | sppgslepka arlppmrres grpikpprkd lpdsqqqhqs skkgklseql khcngilkel |
| 361 | lskkhaayaw pfykpvdasa lglhdyhdii khpmdlstvk rkmenrdyrd aqefaadvrl |
| 421 | mfsncykynp pdhdvvamar klqdvfefry akmpdeplep gplpvstamp pglakssses |
| 481 | sseesssess seeeeeedee deeeeesess dseeerahrl aelqeqlrav heqlaalsqg |
| 541 | piskpkrkre kkekkkkrka ekhrgragad eddkgprapr ppqpkkskka sgsgggsaal |
| 601 | gpsgfgpsgg sgtklpkkat ktappalptg ydseeeeesr pmsydekrql sldinklpge |
| 661 | klgrvvhiiq arepslrdsn peeieidfet lkpstlrele ryvlsclrkk prkpytikkp |
| 721 | vgktkeelal ekkrelekrl qdvsgqlnst kkppkkanek tesssaqqva vsrlsassss |
| 781 | sdssssssss sssdtsdsds g |

BRD2 bromodomain 1 (BRD2_{BD1}) and BRD2 bromodomain 2 (BRD2_{BD2}) are highlighted in bold, wherein L110 in BRD2_{BD1} and L383 in BRD2_{BD2} are underlined.

An exemplary human BRD2 nucleic acid sequence, NCBI Accession No. NM_001113182, version NM_001113182.3, incorporated herein by reference, and which encodes SEQ ID NO: 5, is set forth below:

| (SEQ ID NO: 6) | |
|---|---|
| 1 | gctcgccctc ctccctggcg ctgaccgatg gaccagccgc tccgtgggga ggactccgga |
| 61 | ccctggtggg ggggcggggg ggttctttcg cccccgtggc ggagggcccc tgagaggcgg |
| 121 | atacgggtgt gcctttgggg gtgatgtggc gtgtgggggg aaaggtccga gctcgcctgg |
| 181 | agggggaggg tttttccctt aagtcatccc tcccaggact tgctttttct gctctgagcc |
| 241 | ggacgccgga atggagtttg aggaagagat tcttcgctgc tgctgcctta ccgccgagaa |
| 301 | ccaccacccg ccaggcgtct tgcggccaca cccctggcgg gttcaggcag gctacgccca |
| 361 | cgcgacccct cccgtttccc tgctttggcc aatggaggag ctacgaatgg cacgacctgc |
| 421 | tcgagcttgg cagtctccag ttgggctgtg catggaagct tgggaagact ttgttggaag |
| 481 | gggaggcggg gagagagtgc tggaggctct ggggcgatgg cttccgcacc tcttccaacc |
| 541 | accctctttc cctggagtcg gcggaccaca gctcagccaa ttggcttgga gatgtggcgg |
| 601 | gttgccactt ccctgtgggt ctctgcggca ctcttctgcc tggtgactga caccttggaa |
| 661 | atgaagttta tgacgtcatc gttgcggctg gccaatagaa aaagctcccg cggagaggtg |
| 721 | ttccttcccc ttcgactcag cttcttcacc cgcgtgagcg agcgcgcgcg cgcggagggg |
| 781 | gtggggaaaa gctcaagcag ggtggcgcgc atgagcggcg aagctcctcc tccccgccta |
| 841 | tatataaagg gctggcgcgg ggctcggcgg cgccatttcg tgctggagtg gagcagcctc |
| 901 | tagaacgagc tggaggattc tgcctaccga tacagagcct tcgagtcgtc cggggccgcc |
| 961 | attacaatcc acctccatcc gettggaaat ggccttcgtc ccggcctatg actggtccca |
| 1021 | gcgggcagta cagaccccct agaagcccct ggagctcccc tttttcgggc cccgcccaat |
| 1081 | cctcggagtc tgtccacccc ctctactccg ccctcaagag gatttcaaag atggaggcgg |
| 1141 | cggctcccta aaccactttt cgtgttcatc cgcctccatc cgagatcgaa acgggacctc |
| 1201 | gtcggccccg taggggcccg acaagaagag ggaatccctg cagaccaaca gcgggctata |
| 1261 | ttgacgacgg tgtctgagat cggggaccgt cttttgaaga gtcagtccct ccttagttgc |
| 1321 | ccgcctcagc tgaggccgcc gccattttct tgctgtccgc cgtctgcaga gcgcgccaag |
| 1381 | ctgcccggag ctctccgaga ggccccaaag agactgcttt cgtgccggcc aggcaggggg |
| 1441 | tttgtcgcct ggaggcccaa gaggaacggc ctccccccaa cttagcgggt tatgctggac |
| 1501 | cgggcggtga ggggaaccga ggccacccgg actttccgcg gctgagggca gcgccggttc |
| 1561 | cttgcggtca agatgctgca aaacgtgact ccccacaata agctccctgg ggaagggaat |
| 1621 | gcagggttgc tggggctggg cccagaagca gcagcaccag ggaagaggat tcgaaaaccc |
| 1681 | tctctcttgt atgagggctt tgagagcccc acaatggctt cggtgcctgc tttgcaactt |
| 1741 | acccctgcca acccaccacc cccggaggtg tccaatccca aaaagccagg acgagttacc |
| 1801 | aaccagctgc aatacctaca caaggtagtg atgaaggctc tgtggaaaca tcagttcgca |
| 1861 | tggccattcc ggcagcctgt ggatgctgtc aaactgggtc taccggatta tcacaaaatt |
| 1921 | ataaaacagc ctatggacat gggtactatt aagaggagac ttgaaaacaa ttattattgg |
| 1981 | gctgcttcag agtgtatgca agattttaat accatgttca ccaactgtta catttacaac |
| 2041 | aagcccactg atgatattgt cctaatggca caaacgctgg aaaagatatt cctacagaag |
| 2101 | gttgcatcaa tgccacaaga agaacaagag ctggtagtga ccatccctaa gaacagccac |
| 2161 | aagaaggggg ccaagttggc agcgctccag ggcagtgtta ccagtgccca tcaggtgcct |
| 2221 | gccgtctctt ctgtgtcaca cacagccctg tatactcctc cacctgagat acctaccact |
| 2281 | gtcctcaaca ttccccaccc atcagtcatt tcctctccac ttctcaagtc cttgcactct |
| 2341 | gctggacccc cgctccttgc tgttactgca gctcctccag cccagcccct tgccaagaaa |
| 2401 | aaaggcgtaa agcggaaagc agatactacc acccctacac ctacagccat cttggctcct |
| 2461 | ggttctccag ctagccctcc tgggagtctt gagcctaagg cagcacggct tccccctatg |
| 2521 | cgtagagaga gtggtcgccc catcaagccc ccacgcaaag acttgcctga ctctcagcaa |
| 2581 | caacaccaga gctctaagaa aggaaagctt tcagaacagt taaaacattg caatggcatt |
| 2641 | ttgaaggagt tactctctaa gaagcatgct gcctatgctt ggcctttcta taaaccagtg |
| 2701 | gatgcttctg cacttggcct gcatgactac catgacatca ttaagcaccc catggacctc |
| 2761 | agcactgtca agcggaagat ggagaaccgt gattaccggg atgcacagga gtttgctgct |
| 2821 | gatgtacggc ttatgttctc caactgctat aagtacaatc ccccagatca cgatgttgtg |
| 2881 | gcaatggcac gaaagctaca ggatgtattt gagttccgtt atgccaagat gccagatgaa |
| 2941 | ccactagaac cagggccttt accagtctct actgccatgc cccctggctt ggccaaatcg |
| 3001 | tcttcagagt cctccagtga ggaaagtagc agtgagagct cctctgagga agaggaggag |
| 3061 | gaagatgagg aggacgagga ggaagaagag agtgaaagct cagactcaga ggaagaaagg |
| 3121 | gctcatcgct tagcagaact acaggaacag cttcgggcag tacatgaaca actggctgct |
| 3181 | ctgtcccagg gtccaatatc caagcccaag aggaaaagag agaaaaaaga gaaaaagaag |
| 3241 | aaacggaagg cagagaagca tcgaggccga gctggggccg atgaagatga caaggggcct |
| 3301 | agggcacccc gcccacctca acctaagaag tccaagaaag caagtggcag tgggggtggc |
| 3361 | agtgctgctt taggcccttc tggctttgga ccttctggag gaagtggcac caagctcccc |
| 3421 | aaaaaggcca caaagacagc cccacctgcc ctgcctacag gttatgattc agaggaggag |
| 3481 | gaagagagca ggcccatgag ttacgatgag aagcggcagc tgagcctgga catcaacaaa |
| 3541 | ttacctgggg agaagctggg ccgagttgtg catataatcc aagccaggga gccctcttta |
| 3601 | cgtgattcaa acccagaaga gattgagatt gattttgaaa cactcaagcc atccacactt |
| 3661 | agagagcttg agcgctatgt cctttcctgc ctacgtaaga aaccccggaa gccctacacc |
| 3721 | attaagaagc ctgtgggaaa gacaaaggag gaactggctt tggagaaaaa gcgggaatta |
| 3781 | gaaaagcggt tacaagatgt cagcggacag ctcaattcta ctaaaaagcc ccccaagaaa |
| 3841 | gcgaatgaga aaacagagtc atcctctgca cagcaagtag cagtgtcacg ccttagcgct |
| 3901 | tccagctcca gctcagattc cagctcctcc tcttcctcgt cgtcgtcttc agacaccagt |
| 3961 | gattcagact caggctaagg ggtcaggcca gatggggcag gaaggctccg caggaccgga |
| 4021 | cccctagacc accctgcccc acctgcccct tccccctttg ctgtgacact tcttcatctc |
| 4081 | accccccccc gcccccctct aggagagctg gctctgcagt gggggaggga tgcagggaca |
| 4141 | tttactgaag gagggacatg gacaaaacaa cattgaattc ccagccccat tggggagtga |
| 4201 | tctcttggac acagagcccc cattcaaaat ggggcagggc aagggtggga gtgtgcaaag |
| 4261 | ccctgatctg gagttacctg aggccacagc tgccctattc acttctaagg gccctgtttt |
| 4321 | gagattgttt gttctaattt attttaagct aggtaaggct ggggggaggg tggggccgtg |
| 4381 | gtcccctcag cctccatggg gagggaagaa gggggagctc tttttttacg ttgatttttt |
| 4441 | tttttctact ctgttttccc tttttccttc cgctccattt ggggccctgg gggtttcagt |
| 4501 | catctcccca tttggtcccc tggactgtct ttgttgattc taacttgtaa ataaagaaaa |
| 4561 | tattattcaa gttttgagtt accttaatat ttgcttttgt agtgtttcaa aaggaacatc |
| 4621 | ataagaattg tcttgataat tttgagggaa atattactgc agtgagaaaa ggcaatagct |
| 4681 | aacctataat tggattgtct taatttttaa accagtaggc ttttgctgtg tttttaataa |
| 4741 | agtaaatatg acttttgtaa a |

An exemplary human BRDT amino acid sequence, NCBI Accession No. NP_001229734, version NP_001229734.2, incorporated herein by reference, is set forth below:

| (SEQ ID NO: 7) | |
|---|---|
| 1 | mslpsrqtai ivnppppeyi ntkkngrltn qlqylqkvvl kdlwkhsfsw pfqrpvdavk |
| 61 | lqlpdyytii knpmdlntik krlenkyyak aseciedfnt mfsncylynk pgddivlmaq |
| 121 | aleklfmqkl sqmpqeeqvv gvkerikkgt qqniavssak eksspsatek vfkqqeipsv |
| 181 | fpktsispln vvqgasvnss sqtaaqvtkg vkrkadtttp atsavkasse fsptfteksv |
| 241 | alppikenmp knvlpdsqqq ynvvktvkvt eqlrhcseil kemlakkhfs yawpfynpvd |
| 301 | vnalglhnyy dvvknpmdlg tikekmdnqe ykdaykfaad vrlmfmncyk ynppdhevvt |
| 361 | marmlqdvfe thfskipiep vesmplcyik tditettgre ntneassegn ssddsederv |
| 421 | krlaklqeql kavhqqlqvl sqvpfrklnk kkekskkekk kekvnnsnen prkmceqmrl |
| 481 | kekskrnqpk krkqqfiglk sedednakpm nydekrqlsl ninklpgdkl grvvhiiqsr |
| 541 | epslsnsnpd eieidfetlk astlreleky vsaclrkrpl kppakkimms keelhsqkkq |
| 601 | elekrlldvn nqlnsrkrqt ksdktqpska venvsrlses sssssssses essssdlsss |
| 661 | dssdsesemf pkftevkpnd spskenvkkm knecippegr tgvtqigycv qdttsanttl |
| 721 | vhqttpshvm ppnhhqlafn yqelehlqtv knisplqilp psgdseqlsn gitvmhpsgd |
| 781 | sdttmlesec qapvqkdiki knadswkslg kpvkpsgvmk ssdelfnqfr kaaiekevka |
| 841 | rtqelirkhl eqntkelkas qenqrdlgng ltvesfsnki qnkcsgeeqk ehqqsseaqd |
| 901 | ksklwllkdr dlarqkeqer rrreamvgti dmtlqsdimt mfennfd |

BRDT bromodomain 1 (BRDT_{BD1}) and BRDT bromodomain 2 (BRDT_{BD2}) are highlighted in bold, wherein L63 in BRDT_{BD1} and L306 in BRDT_{BD2} are underlined.

An exemplary human BRDT nucleic acid sequence, NCBI Accession No. NM_001242805, version NM_001242805.2, incorporated herein by reference, and which encodes SEQ ID NO: 7, is set forth below:

| (SEQ ID NO: 8) | |
|---|---|
| 1 | tataaaagcg cctgtcgcgc gaccgccatt acaacaaaaa ctggcggcga ggaactgcgg |
| 61 | agaactgttg ccctgcaccg cttcattttg tgcagcctga aaggggcaat cacataaggt |
| 121 | cggttttgta atatacaggt aacatacagg taatgtaccc tccacagggg gtgctgcctt |
| 181 | aggccggccc cgaggtgcgg ccttgggtgg gaacaaccgt tttttccccc ctctcccctc |
| 241 | cccgacccca tcaacatcgt gcgacagtcc gggcagttga gaaggccacc ccgcttcgtg |
| 301 | cctgcagccc agttcctctt aggtcttgga ctggtgtgga tcgctgctgc ctgtactgta |
| 361 | attttggaaa tgcaggagaa agacatgtac ttgtagacag gattcaaagc agttaagaat |
| 421 | gtctctgcca agtcgacaaa cagctattat tgttaaccct cctccaccag aatatataaa |
| 481 | tactaagaaa aatgggcgat tgacaaatca acttcagtat ctacaaaaag ttgtcctaaa |
| 541 | ggatttatgg aagcatagtt tttcatggcc ctttcaacgt cctgtggatg ctgtgaaact |
| 601 | acagttgcct gattattata ccattataaa aaacccaatg gatttaaata caattaagaa |
| 661 | gcgcttggag aataaatatt atgcgaaggc ttcagaatgt atagaagact tcaatacaat |
| 721 | gttctcaaat tgttatttat ataacaagcc tggagatgac attgttctta tggcacaagc |
| 781 | tctagagaag ctgtttatgc agaaattatc tcagatgcca caagaagagc aagttgtggg |
| 841 | tgttaaggaa agaatcaaga aaggcactca acagaatata gctgtttctt ctgctaaaga |
| 901 | aaaatcatca cccagcgcaa cagaaaaagt atttaagcag caagaaattc cttctgtatt |
| 961 | tcctaagaca tctatttctc ccttgaacgt ggtacaggga gcttcagtca actccagttc |
| 1021 | acaaactgcg gcccaagtta caaaaggtgt gaagaggaaa gcagatacaa caactcctgc |
| 1081 | aacttcagca gttaaagcaa gtagtgaatt ttctccaaca ttcacagaaa aatcagtggc |
| 1141 | actgccacct ataaaagaaa atatgccaaa gaatgttttg ccagattctc agcaacaata |
| 1201 | taatgttgtg aagactgtta aagtaactga acaattaagg cactgtagtg agattcttaa |
| 1261 | agaaatgctt gcaaagaaac atttttcata tgcatggccc ttttataatc ctgttgacgt |
| 1321 | taatgctttg ggactccata actactatga cgttgtcaaa aatccgatgg atcttggaac |
| 1381 | tattaaggag aaaatggata accaagaata taaggatgca tacaaatttg cggcagatgt |
| 1441 | tagattaatg ttcatgaatt gctacaagta caatcctcca gatcacgaag ttgtgacaat |
| 1501 | ggcaagaatg cttcaggatg ttttcgaaac gcatttttca aagatcccga ttgaacctgt |
| 1561 | tgagagtatg cctttatgtt acatcaaaac agatatcaca gaaaccactg gtagagagaa |
| 1621 | cactaatgaa gcctcctctg aagggaactc ttctgatgat tctgaagatg agcgagttaa |
| 1681 | gcgtcttgca aagcttcagg agcagcttaa agctgtacat caacagctcc aggttttgtc |
| 1741 | ccaagtacct ttccgtaagc taaataaaaa gaaagagaag tctaaaaagg aaaagaaaaa |
| 1801 | agaaaaggtt aataacagca atgaaaatcc aagaaaaatg tgtgagcaaa tgaggctaaa |
| 1861 | ggaaaagtcc aagagaaatc agccaaagaa aaggaaacaa cagttcattg gtctaaaatc |
| 1921 | tgaagatgaa gataatgcta aacctatgaa ctatgatgag aaaaggcagt taagtctgaa |
| 1981 | tataaacaaa ctccctggag ataaacttgg gcgagtagtt cacataatac aatcaagaga |
| 2041 | gccttctctg agcaattcca atcctgatga gatagagata gactttgaaa cactgaaagc |
| 2101 | atcaacacta agagaattag aaaaatatgt ttcggcatgt ctaagaaaga gaccattaaa |
| 2161 | acctcctgct aagaaaataa tgatgtccaa agaagaactt cactcacaga aaaaacagga |
| 2221 | attggaaaag cggttactgg atgttaataa tcagttaaat tctagaaaac gtcaaacaaa |
| 2281 | atctgataaa acgcaaccat ccaaagctgt tgaaaatgtt tcccgactga gtgagagcag |
| 2341 | cagcagcagc agcagctcat cagagtctga aagtagcagc agtgacttaa gctcttcaga |
| 2401 | cagcagtgat tctgaatcag aaatgttccc taagtttaca gaagtaaaac caaatgattc |
| 2461 | tccttctaaa gagaatgtaa agaaaatgaa gaatgaatgc ataccgcctg aaggaagaac |
| 2521 | aggcgtcaca cagataggat attgtgtgca agacacaacc tctgccaata ctacccttgt |
| 2581 | tcatcagacc acaccttcac atgtaatgcc accaaatcac caccaattag catttaatta |
| 2641 | tcaagaatta gaacatttac agactgtgaa aaacatttca cctttacaaa ttctgcctcc |
| 2701 | ctcaggtgat tctgaacagc tctcaaatgg cataactgtg atgcatccat ctggtgatag |
| 2761 | tgacacaacg atgttagaat ctgaatgtca agctcctgta cagaaggata taaagattaa |
| 2821 | gaatgcagat tcatggaaaa gtttaggcaa accagtgaaa ccatcaggtg taatgaaatc |
| 2881 | ctcagatgag ctcttcaacc aatttagaaa agcagccata gaaaaggaag taaaagctcg |
| 2941 | gacacaggaa ctcatacgga agcatttgga acaaaataca aaggaactaa aagcatctca |
| 3001 | agaaaatcag agggatcttg ggaatggatt gactgtagaa tctttttcaa ataaaataca |
| 3061 | aaacaagtgc tetggagaag agcagaaaga acatcagcag tcatcagaag ctcaagataa |
| 3121 | atccaaactc tggcttctca aagaccgtga tttagcaagg cagaaagaac aagagaggag |
| 3181 | gaggagagaa gcaatggtgg gtaccattga tatgaccctt caaagtgaca ttatgacaat |
| 3241 | gtttgaaaac aactttgatt aaaactcagt ttttaaatta accatcaact taaaatgaat |
| 3301 | ggtaaaagat caaaatgcat atggtaaaat gattgctttc agataacaag ataccaatct |
| 3361 | tatattgtat tttgactgct ctaaaatgat taaacagttt tcacttacat ttttaaaaaa |
| 3421 | aaaaaaaaaa |

Referring to SEQ ID NO: 1, substitution of a conserved leucine residue L94 and/or L387 (both in bold and underlined) in BRD4 yields non-naturally occurring mutants that may be suitable as dTAGs. In some embodiments, mutants contain conservative substitutions at either or both of these positions, examples of which are L94V, L94G, L94A, L941, L387V, L387G, L387A, L3871, and combinations of such conservative mutations at both positions, e.g., L94V, L387V. Representative examples of dTAGs containing these substitutions and nucleic acids encoding them are as follows:

BRD4_{BD1} 44-168:
(SEQ ID NO: 9)
NPPPPETSNPNKPKRQTNQLQYLLRVVLKTLWKHQFAWPFQQPVDAVKLN
LPDYYKIIKTPMDMGTIKKRLENNYYWNAQECIQDFNTMFTNCYIYNKPG
DDIVLMAEALEKLFLQKINELPTEE;
dTAG BRD4_{BD1} 44-168:
(SEQ ID NO: 10)
NPPPPETSNPNKPKRQTNQLQYLLRVVLKTLWKHQFAWPFQQPVDAVKLN
VPDYYKIIKTPMDMGTIKKRLENNYYWNAQECIQDFNTMFTNCYIYNKPG
DDIVLMAEALEKLFLQKINELPTEE;
BRD4_{BD2} 333-460
(SEQ ID NO: 11)
KDVPDSQQHPAPEKSSKVSEQLKCCSGILKEMFAKKHAAYAWPFYKPVDV
EALGLHDYCDIIKHPMDMSTIKSKLEAREYRDAQEFGADVRLMFSNCYKY
NPPDHEVVAMARKLQDVFEMRFAKMPDE;
dTAGBRD4_{BD2} 333-460:
(SEQ ID NO: 12)
KDVPDSQQHPAPEKSSKVSEQLKCCSGILKEMFAKKHAAYAWPFYKPVDV
EALGVHDYCDIIKHPMDMSTIKSKLEAREYRDAQEFGADVRLMFSNCYKY
NPPDHEVVAMARKLQDVFEMRFAKMPDE;

Referring to SEQ ID NO: 5, substitution of a conserved leucine residue L110 in BRD2_{BD1} or L383 in BRD2_{BD2} or L70 in BRD3_{BD1} or L345 in BRD3_{BD2} or L63 in BRDT_{BD1} or L306 in BRDT_{BD2} yields non-naturally occurring mutants that may be suitable as dTAGs. In some embodiments, mutants contain conservative substitutions at any of these forementioned positions which are L→V, L→G, L→A, and L→I. Representative examples of dTAGs containing L→V substitutions are as follows:

BRD2_{BD1} 71-194
(SEQ ID NO: 13)
KPGRVTNQLQYLHKVVMKALWKHQFAWPFRQPVDAVKLGLPDYHKIIKQP
MDMGTIKRRLENNYYWAASECMQDFNTMFTNCYIYNKPTDDIVLMAQTLE
KIFLQKVASMPQEE;
dTAG BRD2_{BD1} 71-194:
(SEQ ID NO: 14)
KPGRVTNQLQYLHKVVMKALWKHQFAWPFRQPVDAVKLGVPDYHKIIKQP
MDMGTIKRRLENNYYWAASECMQDFNTMFTNCYIYNKPTDDIVLMAQTLE
KIFLQKVASMPQEE;
BRD2_{BD2} 344-455:
(SEQ ID NO: 15)
GKLSEQLKHCNGILKELLSKKHAAYAWPFYKPVDASALGLHDYHDIIKHP
MDLSTVKRKMENRDYRDAQEFAADVRLMFSNCYKYNPPDHDVVAMARKLQ
DVFEFRYAKMPD;
and
dTAG BRD2_{BD2} 344-455:
(SEQ ID NO: 16)
GKLSEQLKHCNGILKELLSKKHAAYAWPFYKPVDASALGVHDYHDIIKHP
MDLSTVKRKMENRDYRDAQEFAADVRLMFSNCYKYNPPDHDVVAMARKLQ
DVFEFRYAKMPD.

Additional dTAG sequences that may be used in the present invention include:

BRD3_{BD1} 24-144:
(SEQ ID NO: 17)
PEVSNPSKPGRKTNQLQYMQNVVVKTLWKHQFAWPFYQPVDAIKLNLPDY
HKIIKNPMDMGTIKKRLENNYYWSASECMQDFNTMFTNCYIYNKPTDDIV
LMAQALEKIFLQKVAQMPQEE;
dTAG BRD3_{BD1} 24-144:
(SEQ ID NO: 18)
PEVSNPSKPGRKTNQLQYMQNVVVKTLWKHQFAWPFYQPVDAIKLNVPDY
HKIIKNPMDMGTIKKRLENNYYWSASECMQDFNTMFTNCYIYNKPTDDIV
LMAQALEKIFLQKVAQMPQEE;
BRD3_{BD2} 306-416:
(SEQ ID NO: 19)
GKLSEHLRYCDSILREMLSKKHAAYAWPFYKPVDAEALELHDYHDIIKHP
MDLSTVKRKMDGREYPDAQGFAADVRLMFSNCYKYNPPDHEVVAMARKLQ
DVFEMRFAKMP;
dTAG BRD3_{BD2} 306-416:
(SEQ ID NO: 20)
GKLSEHLRYCDSILREMLSKKHAAYAWPFYKPVDAEALEVHDYHDIIKHP
MDLSTVKRKMDGREYPDAQGFAADVRLMFSNCYKYNPPDHEVVAMARKLQ
DVFEMRFAKMP;
BRDT_{BD1} 21-137:
(SEQ ID NO: 21)
NTKKNGRLTNQLQYLQKVVLKDLWKHSFSWPFQRPVDAVKLQLPDYYTII
KNPMDLNTIKKRLENKYYAKASECIEDFNTMFSNCYLYNKPGDDIVLMAQ
ALEKLFMQKLSQMPQEE;
dTAG BRDT_{BD1} 21-137:
(SEQ ID NO: 22)
NTKKNGRLTNQLQYLQKVVLKDLWKHSFSWPFQRPVDAVKLQVPDYYTII
KNPMDLNTIKKRLENKYYAKASECIEDFNTMFSNCYLYNKPGDDIVLMAQ
ALEKLFMQKLSQMPQEE;
BRDT_{BD2} 267-377:
(SEQ ID NO: 23)
LRHCSEILKEMLAKKHFSYAWPFYNPVDVNALGLHNYYDVVKNPMDLGTI
KEKMDNQEYKDAYKFAADVRLMFMNCYKYNPPDHEVVTMARMLQDVFETH
F;
and
dTAG BRDT_{BD2} 267-377:
(SEQ ID NO: 24)
LRHCSEILKEMLAKKHFSYAWPFYNPVDVNALGVHNYYDVVKNPMDLGTI
KEKMDNQEYKDAYKFAADVRLMFMNCYKYNPPDHEVVTMARMLQDVFETH
F.

### Methods of Use

The dTAG system, e.g., the dTAG and the bifunctional compound can be utilized to produce a stably expressed fusion protein that includes the dTAG and a protein of interest, which may be an endogenous protein, a mutant form of an endogenous protein or an exogenous (e.g., a therapeutic protein). The fusion protein may be expressed in a cell in vivo, or as the case may be ex vivo or in vitro. As contemplated herein, the 5′- or 3′ in-frame insertion of a nucleic acid sequence encoding a dTAG results, upon expression of the resultant nucleic acid sequence, a protein-dTAG hybrid protein that becomes a target for selective degradation upon contact with (e.g., administration of) a bifunctional compound.

### Proteins of Interest

In some aspects, the methods of the present invention are conducted in vitro. In some embodiments, the method may include genetically modifying a cell by introducing an exogenous nucleic acid comprising a sequence that encodes a dTAG at a genetic locus of an endogenous protein, wherein the thus modified locus expresses the protein with the dTAG; contacting the modified cells with the bifunctional compound of formula I; and detecting a change in a property of the modified cell relative to an unmodified cell to identify protein function.

In some other embodiments, the method includes genetically modifying a cell by introducing an exogenous nucleic acid comprising a sequence that encodes the dTAG fused to the N-terminus or the C-terminus of a mutated version of the endogenous protein; contacting the modified cells with the bifunctional compound of formula I; and determining a change in a property of the modified cell before and after said contacting with the bifunctional compound.

In some embodiments, a nucleic acid encoding a dTAG can be genomically inserted in-frame with a gene encoding a protein or mutant form thereof that is involved in a disease or disorder. Representative examples of particular proteins involved in disorders that may be targeted for dTAG fusion (at the genetic level) include alpha-1 antitrypsin (A1AT), apolipoprotein B (APOB), angiopoietin-like protein 3 (ANGPTL3), proprotein convertase subtilisin/kexin type 9 (PCSK9), apolipoprotein C₃ (APOC3), catenin (CTNNB1), low density lipoprotein receptor (LDLR), C-reactive protein (CRP), apolipoprotein a (Apo(a)), Factor VII, Factor XI, antithrombin III (SERPINC1), phosphatidylinositol glycan class A (PIG-A), C₅, alpha-1 antitrypsin (SERPINA1), hepcidin regulation (TMPRSS6), (delta-aminolevulinate synthase 1 (ALAS-1), acylCaA:diacylglycerol acyltransferase (DGAT), miR-122, miR-21, miR-155, miR-34a, prekallikrein (KLKB1), connective tissue growth factor (CCN2), intercellular adhesion molecule 1 (ICAM-1), glucagon receptor (GCGR), glucocorticoid receptor (GCCR), protein tyrosine phosphatase (PTP-1B), c-Raf kinase (RAF1), fibroblast growth factor receptor 4 (FGFR4), vascular adhesion molecule-1 (VCAM-1), very late antigen-4 (VLA-4), transthyretin (TTR), survival motor neuron 2 (SMN2), growth hormone receptor (GHR), dystrophia myotonic protein kinase (DMPK), cellular nucleic acid-binding protein (CNBP or ZNF9), clusterin (CLU), eukaryotic translation initiation factor 4E (eIF-4e), MDM2, MDM4, heat shock protein 27 (HSP 27), signal transduction and activator of transcription 3 protein (STAT3), vascular endothelial growth factor (VEGF), kinesin spindle protein (KIF11), hepatitis B genome, the androgen receptor (AR), Atonal homolog 1 (ATOH1), vascular endothelial growth factor receptor 1 (FLT1), retinoschism 1 (RS1), retinal pigment epithelium-specific 65 kDa protein (RPE65), Rab escort protein 1 (CHM), and the sodium channel, voltage gated, type X, alpha subunit (PN3 or SCN10A). Additional proteins of interest that may be targeted by dTAG insertion include proteins associated with gain of function mutations, such as for example, cancer causing proteins.

In vivo methods may include genetically modifying a cell by introducing an exogenous nucleic acid comprising a sequence that encodes a dTAG, wherein the thus modified locus expresses the protein with the dTAG as an in-frame N-terminal or C-terminal fusion; introducing the thus modified cells into a non-human animal (e.g., a rodent (e.g., a mouse)); administering to the non-human animal the bifunctional compound of formula I; and detecting a change in a property of the non-human animal relative to an unmodified non-human animal. Thus, the methods described herein may be used to validate a potential protein being targeted as associated with a disease state.

In some embodiments, the cell may be a human cell, e.g., a human cancer cell line or a non-cancerous cell line (for other non-cancerous conditions).

In some embodiments, the methods of the present invention may also be used to provide tunable control of constitutively expressed Clustered Regularly Interspaced Short Palindromic Repeats (CRISPR) associated protein 9 (Cas9) via the degradation of the Cas9-dTAG fused proteins with the bifunctional compound of formula (I). In-frame insertion of the nucleic acid sequence encoding the dTAG with a gene expressing an endogenous protein of interest can be performed or achieved by any known and effective genomic editing processes. In one aspect, the present invention utilizes the CRISPR-Cas9 system to produce knock-in endogenous protein-dTAG fusion proteins that are produced from the endogenous locus and are readily degraded in a ligand-dependent, reversible, and dose-responsive, fashion. In certain embodiments, the CRISPR-Cas9 system is employed in order to insert an expression cassette for dTAG present in a homologous recombination (HR) “donor” sequence with the dTAG nucleic acid sequence serving as a “donor” sequence inserted into the genomic locus of a protein of interest during homologous recombination following CRISPR-Cas endonucleation. The HR targeting vector contains homology arms at the 5′ and 3′ end of the expression cassette homologous to the genomic DNA surrounding the targeting gene of interest locus. By fusing the nucleic acid sequence encoding the dTAG in frame with the target gene of interest, the resulting fusion protein contains a dTAG that is targeted by a bifunctional compound.

The present invention provides for insertion of an exogenous dTAG sequence (also called a “donor sequence” or “donor” or “transgene”) in-frame with the target gene of interest, and the resulting fusion protein contains a dTAG that is targeted by a bifunctional compound. The donor sequence need not be identical to the genomic sequence where it is placed. A donor sequence can contain a non-homologous sequence flanked by two regions of homology to allow for efficient HR at the location of interest. Additionally, donor sequences can contain a vector molecule containing sequences that are not homologous to the region of interest in cellular chromatin. A donor molecule can contain several, discontinuous regions of homology to cellular chromatin. For example, for targeted insertion of sequences not normally present in a region of interest, for example, the dTAGs of the present invention, the sequences can be present in a donor nucleic acid molecule and flanked by regions of homology to sequence in the region of interest. Alternatively, a donor molecule may be integrated into a cleaved target locus via non-homologous end joining (NHEJ) mechanisms. See, e.g., U.S. Patent Application Publications 2011/0207221 and 2013/0326645.

The donor dTAG encoding sequence for insertion can be DNA or RNA, single-stranded and/or double-stranded and can be introduced into a cell in linear or circular form. See, e.g., U.S. Patent Application Publications 2010/0047805 A1, 2011/0281361 A1, and 2011/0207221 A1. The donor sequence may be introduced into the cell in circular or linear form. If introduced in linear form, the ends of the donor sequence can be protected (e.g., from exonucleolytic degradation) by methods known to those of skill in the art.

The donor polynucleotide encoding a dTAG can be introduced into a cell as part of a vector molecule having additional sequences such as, for example, CRISPR-Cas sequences, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The present invention takes advantage of well-characterized insertion strategies, for example the CRISPR-Cas9 system. In general, the “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. (See, e.g., Ruan et al., Sci. Rep. 5:14253 (2015); Park et al., PLoS ONE 9(4): e95101(2014)).

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains). Further included is the donor nucleotide encoding a dTAG for in-frame insertion into the genomic locus of the protein of interest.

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and gRNA (including a fusion of crRNA specific for the target sequence and fixed tracrRNA), and a donor sequence encoding a dTAG are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site, followed by homologous recombination of the donor sequence encoding a dTAG into the genomic locus of a protein of interest, as discussed herein. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced.

In general, a CRISPR system is characterized by elements that promote the formation of a CRISPR complex at the site of a target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex, and wherein insertion of the donor sequence encoding a dTAG is to take place. Full complementarity is not necessarily required, provided there is sufficient complementarity to cause hybridization and promote formation of a CRISPR complex.

Typically, in the context of an endogenous CRISPR system, formation of the CRISPR complex (comprising the guide sequence hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, the tracr sequence, which may comprise or consist of all or a portion of a wildtype tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex.

As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector includes a regulatory element operably linked to an enzyme-coding sequence encoding a CRISPR RNA-guided endonuclease. In some embodiments, a vector includes a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, Cpf1, homologs thereof, or modified versions thereof (See, WO 2015/200334, incorporated herein by reference). These enzymes are known; for example, the amino acid sequence of S. pyogenes Cas9 protein may be found in the SwissProt database under accession number Q99ZW2.

Cas proteins generally comprise at least one RNA recognition or binding domain. Such domains can interact with guide RNAs (gRNAs, described in more detail below). Cas proteins can also comprise nuclease domains, for example endonuclease domains (e.g., DNase or RNase domains), DNA binding domains, helicase domains, protein-protein interaction domains, dimerization domains, and other domains. A nuclease domain possesses catalytic activity for nucleic acid cleavage. Cleavage includes the breakage of the covalent bonds of a nucleic acid molecule. Cleavage can produce blunt ends or staggered ends, and it can be single-stranded or double-stranded.

Any Cas protein that induces a nick or double-strand break into a desired recognition site can be used in the methods and compositions disclosed herein.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina®, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell, and in particular, a protein of interest targeted for controlled degradation through the engineering of an endogenous protein-dTAG hybrid. Exemplary target sequences include those that are unique in the target genome which provide for insertion of the dTAG donor nucleic acid in an in-frame orientation. In some embodiments, a guide sequence is selected to reduce the degree of secondary structure within the guide sequence. Secondary structure may be determined by any suitable polynucleotide folding algorithm.

As described herein, the CRISPR-Cas system is used to insert a nucleic acid sequence encoding a dTAG in-frame with the genomic sequence encoding a protein of interest in a eukaryotic, for example, human cell. In some embodiments, the method entails allowing the CRISPR complex to bind to the genomic sequence of the targeted protein of interest to effect cleavage of the genomic sequence, wherein the CRISPR complex includes the CRISPR enzyme complexed with a guide sequence hybridized to a target sequence within said target polynucleotide, wherein said guide sequence is linked to a tracr mate sequence which in turn hybridizes to a tracr sequence.

In some aspects, the methods include modifying expression of a polynucleotide in a eukaryotic cell by introducing a nucleic acid encoding a dTAG.

In some aspects, the polypeptides of the CRISPR-Cas system and donor sequence are administered or introduced to the cell. The nucleic acids typically are administered in the form of an expression vector, such as a viral expression vector. In some aspects, the expression vector is a retroviral expression vector, an adenoviral expression vector, a DNA plasmid expression vector, or an AAV expression vector. In some aspects, one or more polynucleotides encoding CRISPR-Cas system and donor sequence delivered to the cell. In some aspects, the delivery is by delivery of more than one vectors.

Methods of delivering nucleic acid sequences to cells as described herein are described, for example, in U.S. Pat. Nos. 8,586,526, 6,453,242, 6,503,717, 6,534,261, 6,599,692, 6,607,882, 6,689,558, 6,824,978; 6,933,113, 6,979,539, 7,013,219, and 7,163,824.

The various polynucleotides as described herein may also be delivered using vectors containing sequences encoding one or more of compositions described herein. Any vector systems may be used including plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261, 6,607,882, 6,824,978, 6,933,113, 6,979,539, 7,013,219, and 7,163,824.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787, and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 1991/17424 and WO 1991/16024. Delivery can be to cells (e.g., in vitro or ex vivo administration) or target tissues (e.g., in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRS are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., J. Virol. 176:58-69 (1992); Wilson et al., J. Virol. 63:2374-2378 (1989); Miller et al., J. Virol. 65:2220-2224 (1991); and WO 1994/026877).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 1993/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Trashcan ditties et al., Mol. Cell. Biol. 5:3251-3260 (1985); Tratschin, et al., Mol. Cell. Biol. 4:2072-2081 (1984); Hermonat & Muzyczka, Proc. Natl. Acad. Sci. 81:6466-6470 (1984); and Samulski et al., J. Virol. 63:3822-3828 (1984).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-1023 (1995); Malech et al., Proc. Natl. Acad. Sci. 94(22):12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol. Immunother. 44(1):10-20 (1997); Dranoff et al., Hum. Gene Ther. 1:111-112 (1997)).

Vectors suitable for introduction of polynucleotides described herein also include non-integrating lentivirus vectors (IDLV). See, for example, Naldini et al., Proc. Natl. Acad. Sci. 93:11382-11388 (1996); Dull et al., J. Virol. 72:8463-8471 (1998); Zuffery et al., J. Virol. 72:9873-9880 (1998); Follenzi et al., Nat. Genet. 25:217-222 (2000); and U.S. Patent Application Publication 2009/0117617.

Recombinant adeno-associated virus vectors (rAAV) may also be used to deliver the compositions described herein. All vectors are derived from a plasmid that retains only the AAV inverted terminal repeats flanking the transgene expression cassette. Efficient gene transfer and stable transgene delivery are key features for this vector system. (Wagner et al., Lancet 351(9117): 1702-3 (1998) and Kearns et al., Gene Ther. 9:748-55 (1996)). Other AAV serotypes, including AAV1, AAV3, AAV4, AAV5, AAV6, AAV8, AAV9 and AAVrh10, pseudotyped AAV such as AAV2/8, AAV2/5 and AAV2/6 and all variants thereof, can also be used in accordance with the present invention.

Replication-deficient recombinant adenoviral vectors (Ad) can be produced at high titer and readily infect a number of different cell types. Most adenovirus vectors are engineered such that a transgene replaces the Ad Ela, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-1089 (1998)). Additional examples of the use of adenovirus vectors for gene transfer in clinical trials include Rosenecker et al., Infection 24(1):5-10 (1996); Sterman et al., Hum. Gene Ther. 9(7):1083-1089 (1998); Welsh et al., Hum. Gene Ther. 2:205-218 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and w2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

The vector can be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. 92:9747-9751(1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., antigen-binding fragment (Fab) or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Vectors can be delivered in vivo by administration to an individual subject, typically by systemic administration (e.g., intravenous, intraperitoneal, intramuscular, intrathecal, intratracheal, subdermal, or intracranial infusion) or topical application, as described below. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Vectors (e.g., retroviruses, adenoviruses, liposomes, etc.) containing nucleases and/or donor constructs can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

In some embodiments, the polypeptides of the CRISPR-Cas system are synthesized in situ in the cell as a result of the introduction of polynucleotides encoding the polypeptides into the cell. In some aspects, the polypeptides of the CRISP-Cas system could be produced outside the cell and then introduced thereto. Methods for introducing a CRISPR-Cas polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods, as described herein. Preferably, the CRISPR-Cas polynucleotide is transiently expressed and not integrated into the genome of the cell. In some embodiments, the CRISPR-Cas polynucleotides may be introduced into the cell by for example, recombinant viral vectors (e.g., retroviruses, adenoviruses), liposome and the like. For example, in some aspects, transient transformation methods include microinjection, electroporation, or particle bombardment. In some embodiments, the CRISPR-Cas polynucleotides may be included in vectors, more particularly plasmids or virus, in view of being expressed in the cells.

In some embodiments, non-CRISPR-CAS viral and non-viral based gene transfer methods can be used to insert nucleic acids encoding a dTAG in frame in the genomic locus of a protein of interest in mammalian cells or target tissues. Such methods can be used to administer nucleic acids encoding components of a zing finger protein a zing finger nuclease (ZFN), transcription activator-like effector protein (TALE), and/or transcription activator-like effector nuclease (TALEN) system to cells in culture, or in a host organism including a donor sequence encoding a dTAG for in-frame insertion into the genomic locus of a protein of interest.

Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11: 162-166 (1993); Dillon, TIBTECH 11: 167-173 (1993); Miller, Nature 357:455-460 (1992); Van Brunt, Biotechnology 6(10):1149-1154 (1988); Vigne, Restor. Neurol. Neurosci. 8:35-36 (1995); Kremer & Perricaudet, British Medical Bulletin 51(1):31-44 (1995); and Yu et al., Gene Therapy 1:13-26 (1994).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); and U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGenelC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see, MacDiarmid et al., Nat. Biotechnol. 27(7):643 (2009).

### Chimeric Antigen Receptor Protein (CAR)-dTAG Fusion Proteins

In some embodiments, the methods may be used in connection with immunotherapy (e.g., clinically), and include administering a subject with immune effector cells such as autologous or allogeneic T-cells (CAR-T cells) which have been genetically modified to express a CAR-dTAG fusion protein. In the event the patient experiences an adverse immune response (e.g., cytokine release syndrome or neurotoxicity) as a result of the therapy, the patient may then be administered the bifunctional compound which will result in degradation of the CAR-dTAG fusion protein.

Genetically modified T cells expressing chimeric antigen receptors (CAR-T therapy) have shown to have therapeutic efficacy in a number of cancers, including lymphoma (Till et al., Blood 119:3940-50 (2012)), chronic lymphocytic leukemia (Porter et al., N. Engl. J. Med. 365:725-33 (2011)), acute lymphoblastic leukemia (Grupp et al., N. Engl. J. Med. 368:1509-18 (2013)) and neuroblastoma (Louis et al., Blood 118:6050-56 (2011)). Five autologous CAR-T cell therapies (Kymriah™, Yescarta™, Abecma®, Breyanzi®, and Tecartus™) have been approved by the FDA.

CAR-T therapy is not, however, without significant side effects. Although most adverse events with CAR-T are tolerable and acceptable, the administration of CAR-T cells has, in a number of cases, resulted in severe systemic inflammatory reactions, including cytokine release syndrome and tumor lysis syndrome (Xu et al., Leukemia Lymphoma 54:255-60 (2013)).

Cytokine release syndrome (CRS) is an inflammatory response clinically manifesting with fever, nausea, headache, tachycardia, hypotension, hypoxia, as well as cardiac and/or neurologic manifestations. Severe cytokine release syndrome is described as a cytokine storm, and can be fatal. CRS is believed to be a result of the sustained activation of a variety of cell types such as monocytes and macrophages, T cells and B cells, and is generally characterized by an increase in levels of TNFα and IFNγ within 1 to 2 hours of stimulus exposure, followed by increases in interleukin (IL)-6 and IL-10 and, in some cases, IL-2 and IL-8 (Doessegger et al., Nat. Clin. Transl. Immuno. 4:e39 (2015)).

Tumor lysis syndrome (TLS) is a metabolic syndrome that is caused by the sudden killing of tumor cells with chemotherapy, and subsequent release of cellular contents with the release of large amounts of potassium, phosphate, and nucleic acids into the systemic circulation. Catabolism of the nucleic acids to uric acid lease to hyperuricemia; the marked increase in uric acid excretion can result in the precipitation of uric acid in the renal tubules and renal vasoconstriction, impaired autoregulation, decreased renal flow, oxidation, and inflammation, resulting in acute kidney injury. Hyperphosphatemia with calcium phosphate deposition in the renal tubules can also cause acute kidney injury. High concentrations of both uric acid and phosphate potentiate the risk of acute kidney injury because uric acid precipitates more readily in the presence of calcium phosphate and vice versa that results in hyperkalemia, hyperphosphatemia, hypocalcemia, uremia, and acute renal failure. It usually occurs in patients with bulky, rapidly proliferating, treatment-responsive tumors (Wintrobe et al., “Complications of hematopoietic neoplasms,” in Wintrobe's Clinical Hematology, 11^{th} ed., Lippincott Williams & Wilkins, Vol. II, 1919-44 (2003)).

The dramatic clinical activity of CAR-T cell therapy necessitates the need to implement safety strategies to rapidly reverse or abort the T cell responses in patients undergoing CRS or associated adverse events.

Accordingly, the present invention includes fusion proteins that are CARs containing the dTAG. The CARs of the present invention further include an extracellular ligand binding domain capable of binding to an antigen, a transmembrane domain, and an intracellular domain in this order from the N-terminal side, wherein the intracellular domain includes at least one signaling domain. The dTAG may be located at the N-terminus or between the extracellular binding domain and the transmembrane domain, provided that there is no disruption to antigen binding or insertion into the membrane. Similarly, the dTAG can be located at the C-terminus, between the transmembrane domain and the intracellular domain or between signaling domains when more than one is present, provided that there is no disruption to intracellular signaling or insertion into the membrane. The dTAG is preferably located at the C-terminus.

In some embodiments, the fusion protein is the CAR used in tisagenlecleucel (Kymriah™) immunotherapy plus the dTAG described herein. Tisagenlecleucel is genetically modified, antigen-specific, autologous T cells that target CD19. The extracellular domain of the CAR is a murine anti-CD19 single chain antibody fragment (scFv) from murine monoclonal FMC63 hybridoma. The intracellular domain of the CAR is a T cell signaling domain derived from human CD3ξ and a co-stimulatory domain derived from human 4-1BB (CD137). The transmembrane domain and a spacer, located between the scFv domain and the transmembrane domain, are derived from human CD8α. Kymriah™ (tisagenlecleucel) is approved for the treatment of patients up to 25 years of age with B-cell precursor acute lymphoblastic leukemia (ALL) that is refractory or in relapse (R/R) and for the treatment of adults with R/R diffuse large B-cell lymphoma (DLBCL), the most common form of non-Hodgkin's lymphoma, as well as high grade B-cell lymphoma and DLBCL arising from follicular lymphoma.

In some embodiments, the fusion protein is the CAR used in axicabtagene ciloleucel (Yescarta™) immunotherapy plus the dTAG described herein. Axicabtagene ciloleucel is genetically modified, antigen-specific, autologous T cells that target CD19. The extracellular domain of the CAR is a murine anti-CD19 single chain antibody fragment (scFv). The intracellular domain of the CAR is two signaling domains, one derived from human CD3ξ and one derived from human CD28. Yescarta™ (axicabtagene ciloleucel) is approved for the treatment of adults with R/R large B cell lymphoma including DLBCL not otherwise specified, primary mediastinal large B-cell lymphoma, high grade B-cell lymphoma, and DLBCL arising from follicular lymphoma.

In some embodiments, the fusion protein is the CAR used in idecabtagene vicleucel (Abecma®) immunotherapy plus the dTAG described herein. Idecabtagene vicleucel is genetically modified, antigen-specific, autologous T cells that target B-cell maturation antigen (BCMA). The extracellular domain of the CAR is a murine anti-BCMA single chain antibody fragment (scFv). The intracellular domain of the CAR is two signaling domains, one derived from human CD3ξ and one derived from human CD28. Abecma® (idecabtagene vicleucel) is approved for the treatment of adults with relapsed or refractory multiple myeloma after four or more prior lines of therapy, including an immunomodulatory agent, a proteasome inhibitor, and an antidCD38 monoclonal antibody.

In some embodiments, the fusion protein is the CAR used in lisocabtagene maraleucel (Breyanzi®) immunotherapy plus the dTAG described herein. Lisocabtagene maraleucel is genetically modified, antigen-specific, autologous T cells that target CD19. The extracellular domain of the CAR is a murine anti-CD19 single chain antibody fragment (scFv). The intracellular domain of the CAR is two signaling domains, one derived from human CD3 and one derived from human CD28. Breyanzi® (lisocabtagene maraleucel) is approved for the treatment of adults with relapsed or refractory (R/R) large B-cell lymphoma after two or more lines of systemic therapy, including diffuse large B-cell lymphoma (DLBCL) not otherwise specified (including DLBCL arising from indolent lymphoma), high-grade B-cell lymphoma, primary mediastinal large B-cell lymphoma, and follicular lymphoma grade 3B.

In some embodiments, the fusion protein is the CAR used in brexucabtagene autoleucel (Tecartus™) immunotherapy plus the dTAG described herein. Brexucabtagene autoleucel is genetically modified, antigen-specific, autologous T cells that target CD19. The extracellular domain of the CAR is a murine anti-CD19 single chain antibody fragment (scFv). The intracellular domain of the CAR is two signaling domains, one derived from human CD3ξ and one derived from human CD28. Tecartus™ (brexucabtagene autoleucel) is approved for the treatment of adults with relapsed or refractory mantle cell lymphoma (MCL).

The nucleic acid encoding the CAR can be easily prepared from an amino acid sequence of the specified CAR by a conventional method. A base sequence encoding an amino acid sequence can be readily obtained from, for example, the aforementioned amino acid sequences or publicly available reference sequences, for example, NCBI RefSeq IDs or accession numbers of GenBank, for an amino acid sequence of each domain, and the nucleic acid of the present invention can be prepared using a standard molecular biological and/or chemical procedure. RefSeq IDs for commonly used CAR domains are known in the art, for example, U.S. Pat. No. 9,175,308 discloses a number of specific amino acid sequences particularly used as CAR transmembrane and intracellular signaling domains. As one example, based on the base sequence, a nucleic acid can be synthesized, and the nucleic acid of the present invention can be prepared by combining DNA fragments which are obtained from a cDNA library using a polymerase chain reaction (PCR).

Immune effector cells expressing the CAR of the present invention can be engineered by introducing the nucleic acid encoding a CAR described above into a cell. In one embodiment, the step is carried out ex vivo. For example, a cell can be transformed ex vivo with a vector carrying the nucleic acid of the present invention to produce a cell expressing the CAR of the present invention.

Representative examples of immune effector cells as described herein include cytotoxic lymphocytes, T-cells, cytotoxic T-cells, T helper cells, Th17 T-cells, natural killer (NK) cells, natural killer T (NKT) cells, mast cells, dendritic cells, killer dendritic cells, or B cells derived from a mammal, for example, a human cell, or a cell derived from a non-human mammal such as a monkey, a mouse, a rat, a pig, a horse, or a dog. For example, a cell collected, isolated, purified or induced from a body fluid, a tissue or an organ such as blood (peripheral blood, umbilical cord blood etc.) or bone marrow can be used. A peripheral blood mononuclear cell (PBMC), an immune cell (a dendritic cell, a B cell, a hematopoietic stem cell, a macrophage, a monocyte, a NK cell or a hematopoietic cell (a neutrophil, a basophil)), an umbilical cord blood mononuclear cell, a fibroblast, a precursor adipocyte, a hepatocyte, a skin keratinocyte, a mesenchymal stem cell, an adipose stem cell, various cancer cell strains, or a neural stem cell can be used. In the present invention, use of a T-cell, a precursor cell of a T-cell (a hematopoietic stem cell, a lymphocyte precursor cell etc.) or a cell population containing them is preferable. Representative examples of T-cells include CD8-positive T-cells, CD4-positive T-cells, regulatory T-cells, cytotoxic T-cells, and tumor infiltrating lymphocytes. The cell population containing a T-cell and a precursor cell of a T-cell includes a PBMC. The aforementioned cells may be collected from a living body, obtained by expansion culture of a cell collected from a living body, or established as a cell strain. When transplantation of the produced CAR-expressing cell or a cell differentiated from the produced CAR-expressing cell into a living body is desired, it is preferable to introduce the nucleic acid into a cell collected from the living body itself or a conspecific living body thereof. Thus, the immune effector cells may be autologous or allogeneic.

The cell expressing the CAR can be used as a therapeutic agent for a disease. The therapeutic agent can be the cell expressing the CAR as an active ingredient, and may further include a suitable excipient. The disease against which the cell expressing the CAR is administered is not limited as long as the disease shows sensitivity to the cell. Representative examples of diseases treatable with cells expressing CARs of the present invention include a cancer (blood cancer (leukemia), solid tumor, etc.), an inflammatory disease/autoimmune disease (asthma, eczema), hepatitis, and an infectious disease, the cause of which is a virus such as influenza and HIV, a bacterium, or a fungus, for example, tuberculosis, MRSA, VRE, and deep mycosis. The cell expressing the CAR of the present invention that binds to an antigen possessed by a cell that is desired to be decreased or eliminated for treatment of the aforementioned diseases, that is, a tumor antigen, a viral antigen, a bacterial antigen or the like is administered for treatment of these diseases. The cell of the present invention can also be utilized for prevention of an infectious disease after bone marrow transplantation or exposure to radiation, donor lymphocyte transfusion for the purpose of remission of recurrent leukemia, and the like. The therapeutic agent including the cell expressing the CAR as an active ingredient can be administered intradermally, intramuscularly, subcutaneously, intraperitoneally, intranasally, intraarterially, intravenously, intratumorally, or into an afferent lymph vessel, by parenteral administration, for example, by injection or infusion, although the administration route is not limited.

In some embodiments, the antigen binding moiety portion of the CAR of the invention is designed to treat a particular cancer (e.g., hematological cancer). For example, a CAR designed to target CD19 can be used to treat cancers and disorders including pre-B ALL (pediatric indication), adult ALL, mantle cell lymphoma, diffuse large B-cell lymphoma, and salvage post allogenic bone marrow transplantation.

In some embodiments, the antigen binding moiety portion of the CAR of the invention may be used in the treatment of solid tumors (e.g., sarcomas, carcinomas, and lymphomas).

When “an immunologically effective amount”, “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). In some embodiments, the CAR expressing cells described herein may be administered at a dosage of 10⁴ to 10⁹ cells/kg body weight, preferably 10⁵ to 10⁶ cells/kg body weight, including all integer and non-integer values within those ranges. T-cell compositions may also be administered multiple times at these dosages. The cells can be administered by using infusion techniques that are commonly known in immunotherapy (see, e.g., Rosenberg et al., New Eng. J. Med. 319:1676 (1988)). The optimal dosage and treatment regime for a particular patient can readily be determined by one skilled in the art of medicine by monitoring the patient for signs of disease and adjusting the treatment accordingly.

The administration of the CAR expressing cells may be carried out in any convenient manner, including by aerosol inhalation, injection, ingestion, transfusion, implantation or transplantation. The CAR expressing cells described herein may be administered to a patient subcutaneously, intradermally, intratumorally, intranodally, intramedullary, intramuscularly, by intravenous (i.v.) injection, or intraperitoneally. In one embodiment, the CAR expressing cells of the present invention are administered to a patient by intradermal or subcutaneous injection. In another embodiment, the CAR expressing cells of the present invention are preferably administered by i. v. injection. The CAR expressing cells may be injected directly into a tumor, lymph node, or site of infection.

Further features of CAR proteins, nucleic acids encoding CAR proteins, immune effector cells expressing CARs and methods of using CAR expressing cells for the treatment of diseases are disclosed in U.S. Patent Application Publication 2018/0169109.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

The modes of administration (e.g., oral, parenteral) may also be determined in accordance with the standard medical practice.

### Dosage Amounts of Bifunctional Compounds of Formula (I)

For purposes of pre-clinical studies, amounts of the bifunctional compounds may be readily determined by persons skilled in the art to obtain the desired therapeutic effect. In general, amounts range from about 0.1 nM to about 1 mM, and in some embodiments from about 0.1 nM to about 100 μM, and in some embodiments, from about 0.01 nM to about 10 μM, and in some embodiments, from about 1 nM to about 1 μM, and in some embodiments from about 10 nM to about 1 μM. In some embodiments, the amounts range from 1 nM to about 1 μM.

With respect to clinical uses, as used herein, the term, “therapeutically effective amount” or “effective amount” refers to an amount of the bifunctional compound of formula I or a pharmaceutically acceptable salt or a stereoisomer thereof; or a composition including the bifunctional compound of formula I or a pharmaceutically acceptable salt or a stereoisomer thereof, effective in producing the desired therapeutic response. The term “therapeutically effective amount” includes the amount of the bifunctional compound or a pharmaceutically acceptable salt or a stereoisomer thereof, when administered, may induce selective degradation of an endogenous or exogenous protein of interest. In the context of CAR-T therapy, the amount of the bifunctional compound is effective to degrade the CAR in the immune effector cells which in turn, may reduce or alleviate to some extent an adverse immune response, e.g., cytokine release syndrome (CRS) or a metabolic syndrome, e.g., tumor lysis syndrome (TLS).

In respect of the therapeutic amount of the bifunctional compound of formula I, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed and the particular pharmaceutically acceptable carrier utilized.

The total daily dosage of the bifunctional compounds of this invention and usage thereof may be decided in accordance with standard medical practice, e.g., by the attending physician using sound medical judgment. The specific therapeutically effective dose for any particular subject will depend upon a variety of factors including the disease or disorder being treated and the severity thereof (e.g., its present status); the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, The Pharmacological Basis of Therapeutics, 10th ed., A. Gilman, J. Hardman and L. Limbird, eds., McGraw-Hill Press, 155-173, 2001).

In some embodiments, the bifunctional compound may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. Dosages smaller than 0.001 mg/kg or greater than 50 mg/kg (for example 50-100 mg/kg) may be administered to a subject. In some embodiments, the bifunctional compound may be administered at dosage levels of about 100 mg/kg.

### Pharmaceutical Kits

The present compositions and genetically modified cells may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a bifunctional compound of the present invention or a pharmaceutical composition. The kits may include the bifunctional compound of formula I or a pharmaceutically acceptable salt or stereoisomer thereof, optionally with nucleic acid encoding the dTAG in a separate container. The kits or pharmaceutical systems of the invention may also include printed instructions for using the bifunctional compounds, compositions and nucleic acids encoding the dTAGs.

These and other aspects of the present invention will be further appreciated upon consideration of the following Examples, which are intended to illustrate certain particular embodiments of the invention but are not intended to limit its scope, as defined by the claims.

### EXAMPLES

### Example 1: Design of Selective Degrader Molecules Using the Bump-and-Hole Strategy

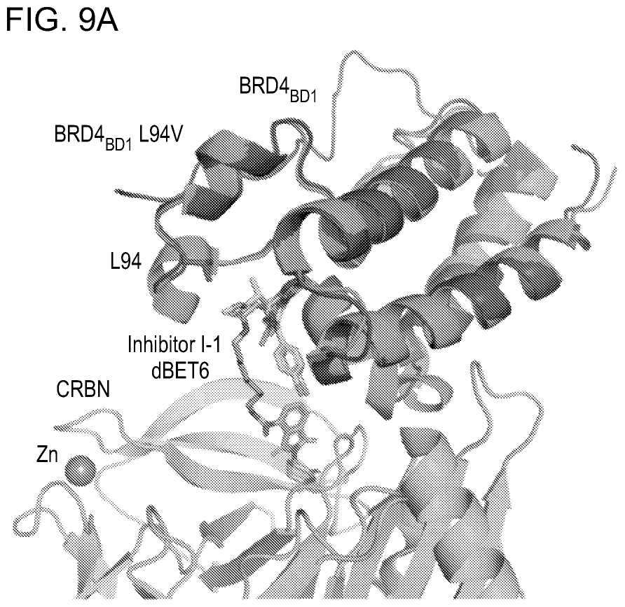

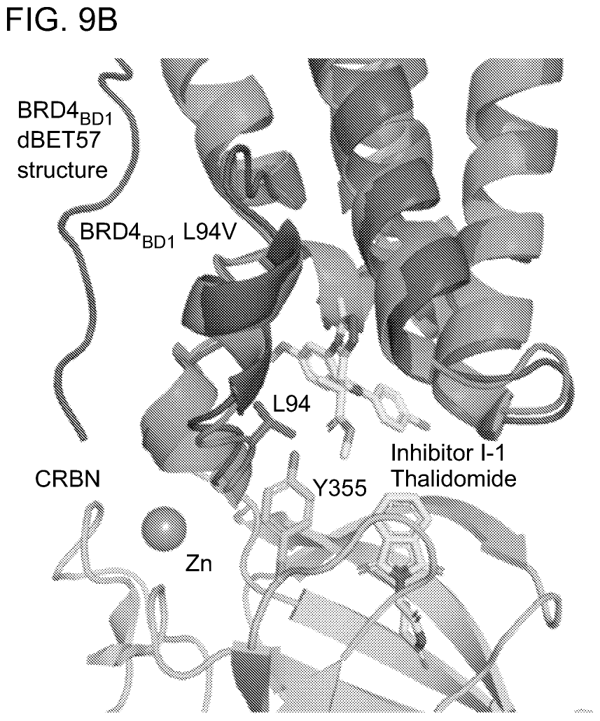

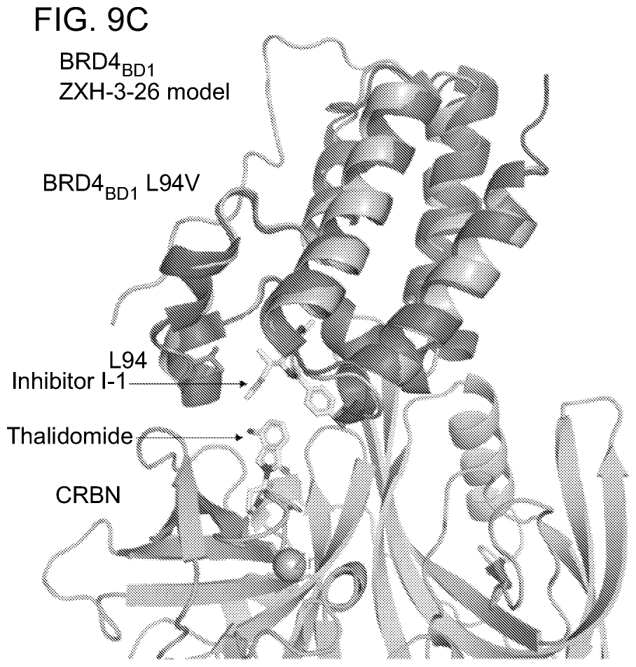

In order to maximize chances of generating an active bifunctional compound or degrader, the designs were based on existing structure-activity relationship (SAR) of CRBN based BET bromodomain of known degraders dBET6, dBET57 and ZXH-3-2622 (FIG. 1A) (Nowak et al., Nat Chem Biol 14 (7):706-714 (2018)). These three degraders utilized the same target binder for BET bromodomain and CRBN, with different linker and exit vector designs. The crystal structure of BRD4_{BD1}L94V in complex with the ester analog I-1 was aligned to the corresponding bromodomain in the ternary complex structure of DDB1ΔB-CRBN-dBET6-BRD4_{BD1}, in which dBET6 and I-1 show close overlap (FIG. 9A). In addition, L94V, conserved across BET family of bromodomains, was positioned far from the potential dimerization interface (FIG. 9C, FIG. 1B). Degrader dBET57 recruits BRD4 with a distinct binding mode and structural alignment of BRD4_{BD1}L94V, indicating that L94V would place itself in close proximity to the CRBN Y355 (FIG. 1C). The putative model for the conformation of the selective BRD4_{BD1} degrader ZXH-3-26 was also aligned, in which no dimerization interface was shown to interfere with BRD4_{BD1}L94V (FIG. 9C). Therefore, it was determined that designs based on dBET6 analogs would be the most promising starting points. Based on the structure alignments and synthetic tractability, a series of molecules were synthesized with linker lengths ranging from C5 to C9, as well as PEG1 and PEG2 (bifunctional compounds 1-5) as the candidate degraders for the BRD4_{BD1}L94V degradation tag. See, examples 2-6.

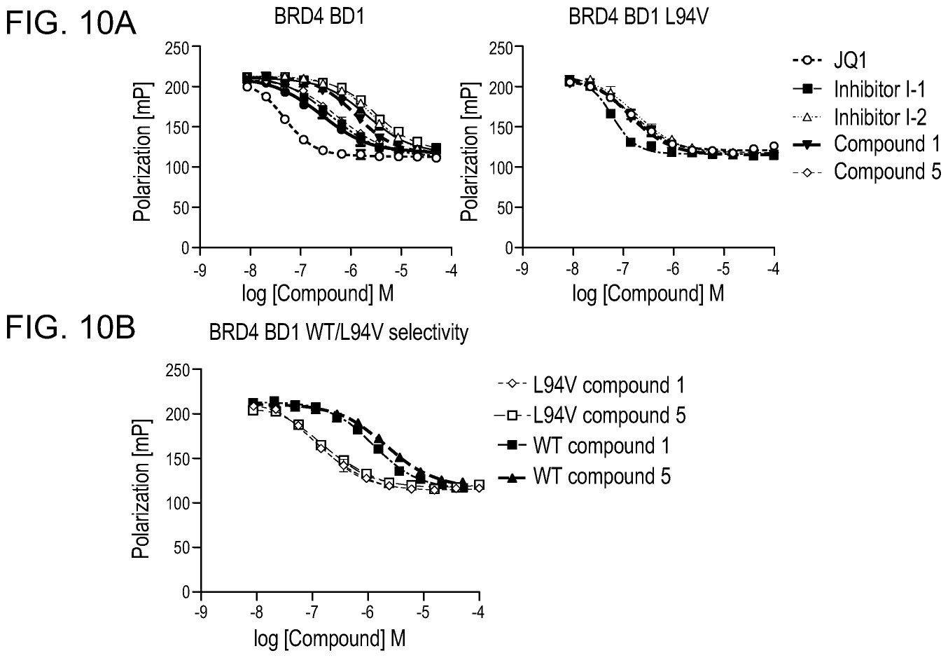

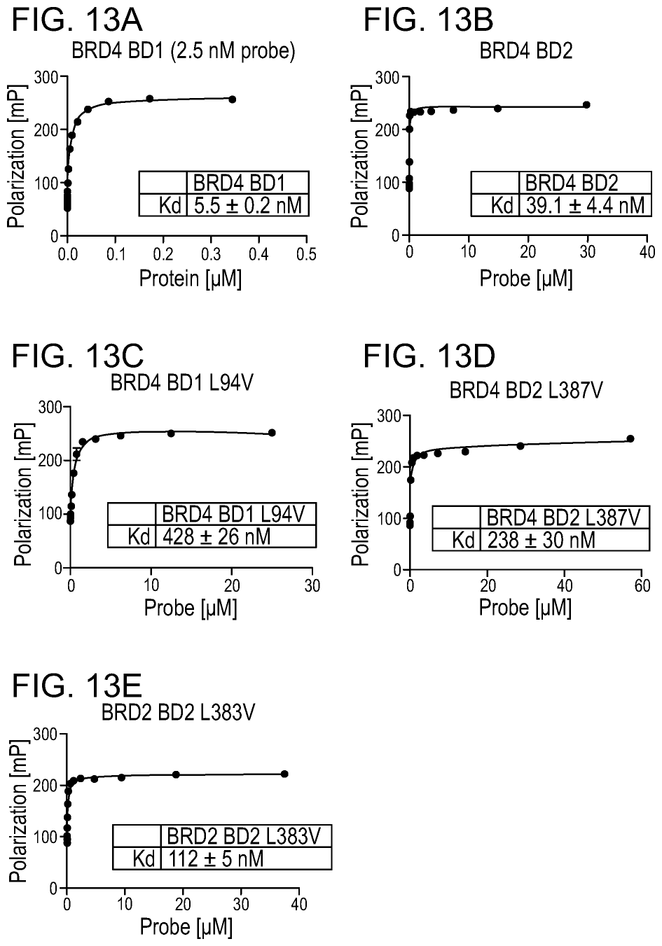

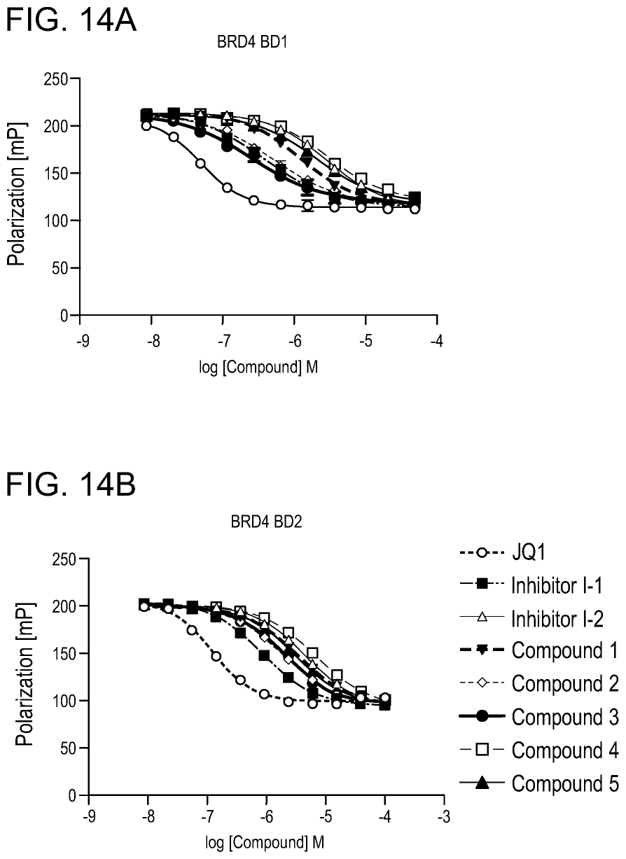

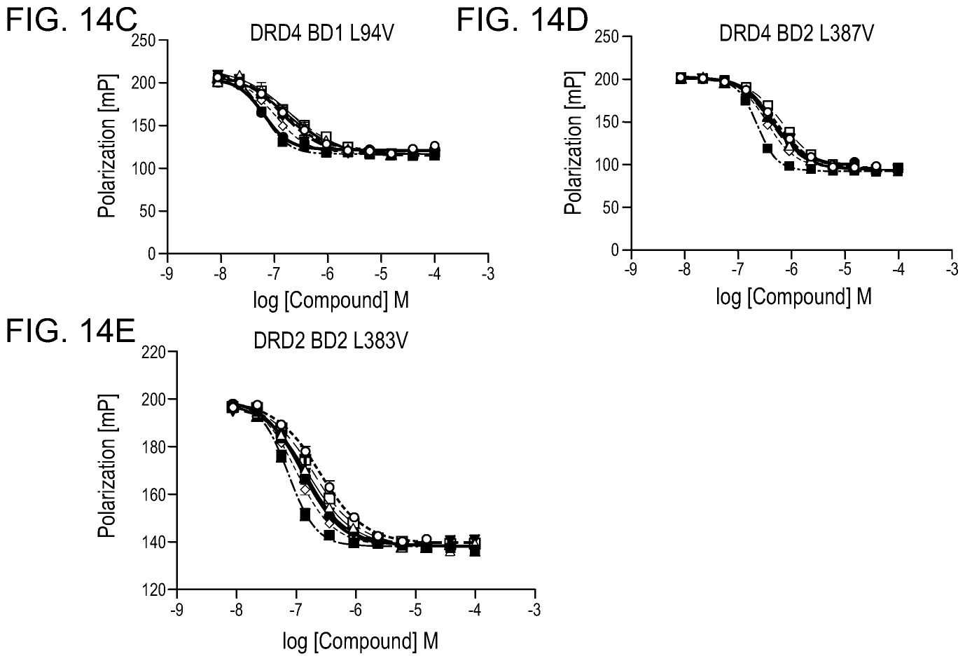

In order to assess the ability of parental compounds and their degrader analogs to bind to wild type BRD4 as well as the BRD4 mutants, a competitive fluorescence polarization binding assay based on displacement of a JQ1-fluorescein isothiocyanate (FITC) probe was established. BRD4_{BD2} and their corresponding hole-modified mutants BRD4_{BD1}L94V, BRD4_{BD2}L387V, and BRD2_{BD2}L383V were titrated into 10 nM JQ1-FITC fluorescent probe. The binding assay for BRD4_{BD1} required lowering the JQ1-FITC probe concentration to 2.5 nM to increase sensitivity range. These binding assays resulted in apparent dissociation constants (K_{d,app}) of 5.5±0.2 nM for BRD4_{BD1}, 428±126 nM for BRD4_{BD1}L94V mutant, 39±4.4 nM for BRD4_{BD2}, and 238±30 nM for BRD4_{BD2}L387V, indicating that mutant versions of the BRD4 bromodomain have approximately a 70-fold loss in affinity as compared to the JQ1-FITC probe (FIG. 13A-FIG. 13E). We next performed competitive titration of parental inhibitors as well as degraders to preformed complexes of bromodomains and JQ1-FITC probe was then (Table 1, FIG. 10A-FIG. 10B, FIG. 14A-FIG. 14E). The results show potent binding of JQ1 and bumped analogs (inhibitors I-1 and 12), as well as bifunctional degraders (Table 1, Supp. FIG. 2C). Notably, inhibitor I-1 was 3-fold more selective in binding BRD2_{BD2} L383V than JQ1 (Table 1, FIG. 14A-FIG. 14E). Degraders derived from inhibitors I-1 and I-2 showed more potent binding to BRD2_{BD2}L383V than the JQ1. Both inhibitors I-1 and I-2 have significantly reduced affinity to wild type BRD4_{BD2} with IC₅₀ values ranging from 0.8 to 4.5 μM (Table 1, FIG. 14A-FIG. 14E). The same was observed for the degrader molecules exemplified in the inventive bifunctional compounds with IC₅₀ values ranging from 2.0 μM for compound 2 to 7.0 μM for compound 4. As expected, JQ1 was able to potently bind BRD4_{BD2} with an IC₅₀ value of about 123 nM. In conclusion, significantly weaker binding of bumped ligands to the wild type bromodomains was observed, in line with previously described SAR (Baud et al., Science 346(6209):638-641 (2014); Baud et al., J Med Chem 59(4):1492-500 (2016); Runcie et al., Chem Sci 9(9):2452-2468 (2018).

The structures of known degraders dBET6 and dBET57, and inhibitors I-1 and I-2 are shown below:

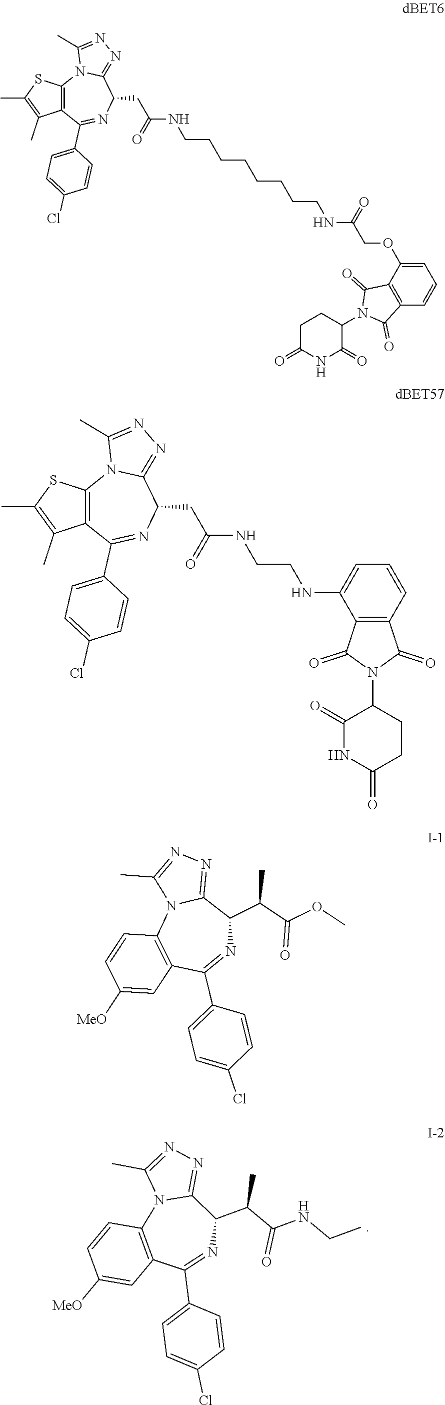 [SMILES: Cc1sc2c(c1C)C(c1ccc(Cl)cc1)=N[C@@H](CC(=O)NCCCCCCCCNC(=O)COc1cccc3c1C(=O)N(C1CCC(=O)NC1=O)C3=O)c1nnc(C)n1-2 ; Cc1sc2c(c1C)C(c1ccc(Cl)cc1)=N[C@@H](CC(=O)NCCNc1cccc3c1C(=O)N(C1CCC(=O)NC1=O)C3=O)c1nnc(C)n1-2 ; COC(=O)[C@H](C)[C@@H]1N=C(c2ccc(Cl)cc2)c2cc(OC)ccc2-n2c(C)nnc21 ; CCNC(=O)[C@H](C)[C@@H]1N=C(c2ccc(Cl)cc2)c2cc(OC)ccc2-n2c(C)nnc21]

FITC-conjugated JQ1 (FITC-JQ1) (10 nM final concentration) in assay buffer containing 50 mM Tris pH 7.5, 200 mM NaCl, 0.1% Pluronic® F-68 solution (Sigma) was mixed with increasing concentration of purified his₆-BRD4_{BD2}, his₆-BRD4_{BD1}L94V, BRD4_{BD2}L387V or BRD2_{BD2}L383V (2-fold, 23-point dilution and DMSO control) in 384-well microplates (Corning®, 4514) and incubated for 15 min at RT. Titration for his₆-BRD4_{BD1} was performed as described in example 7, below, but using buffer containing 2.5 nM final concentration of the FITC-conjugated JQ1 probe. The change in fluorescence polarization was monitored using a PHERAstar® FS microplate reader (BMG Labtech). The K_{d} were obtained from a fit of ‘one-site total’ model in GraphPad Prism 9 from three independent replicates (N=3).

Compounds in FITC-conjugated JQ1 displacement assay were dispensed in a 384-well microplate (Corning™, 4514) using D300e Digital Dispenser (HP) normalized to 1% DMSO into 10 nM FITC-JQ1, 50 mM Tris pH 7.5, 200 mM NaCl, 0.1% Pluronic® F-68 solution (Sigma®) with 400 nM BRD4_{BD1}L94V or 250 nM BRD4_{BD2}L387V or 150 nM BRD2_{BD2}L383V or 50 nM BRD4_{BD2}. Assay for his₆-BRD4_{BD1} was performed as described above, but with 10 nM his₆-BRD4_{BD1} protein and 2.5 nM FITC-JQ1 probe. The change in fluorescence polarization was monitored using a PHERAstar® FS microplate reader (BMG Labtech) until a stable signal was observed. Dose response data (N=3) was plotted and IC₅₀ values were estimated using variable slope equation in GraphPad Prism 9.

TABLE 1
IC₅₀ values in nM from competitive binding assays.

| | JQ1 | I-1 | I-2 | 1 | 2 | 3 | 4 | 5 |
|---|---|---|---|---|---|---|---|---|
| BRD4 | 49 ± 4 | 39 ± 24 | 2870 ± 140 | 1260 ± 60 | 525 ± 39 | 260 ± 32 | 3130 ± 160 | 2080 ± 140 |
| BD1 | | | | | | | | |
| BRD4 | 149 ± 13 | 62 ± 4 | 187 ± 20 | 132 ± 10 | 98 ± 6 | 63 ± 4 | 218 ± 19 | 168 ± 18 |
| BD1 | | | | | | | | |
| L94V | | | | | | | | |
| BRD4 | 523 ± 15 | 231 ± 4 | 495 ± 11 | 438 ± 8 | 354 ± 9 | 464 ± 26 | 686 ± 12 | 464 ± 12 |
| BD2 | | | | | | | | |
| L387V | | | | | | | | |
| BRD2 | 246 ± 15 | 74 ± 3 | 160 ± 9 | 145 ± 7 | 103 ± 6 | 130 ± 6 | 196 ± 9 | 143 ± 6 |
| BD2 | | | | | | | | |
| L383V | | | | | | | | |
| BRD4 | 123 ± 7 | 863 ± 19 | 4650 ± 180 | 3505 ± 110 | 1990 ± 86 | 2240 ± 200 | 6970 ± 300 | 3040 ± 89 |
| BD2 | | | | | | | | |

Compounds were titrated into FITC-JQ1 probe and wild type or mutant protein, N=3. Values are shown as IC₅₀±S.E. (standard error).

### Example 2: Synthesis of (2R)-2-((4S)-6-(4-chlorophenyl)-8-methoxy-1-methyl-4H-benzo[f][1,2,4]triazolo [4,3-a][1,4]diazepin-4-yl)-N-(5-(2-((2-(2,6-dioxopiperidin-3-yl)-1,3-dioxoisoindolin-4-yl)oxy)acetamido)pentyl)propanamide (1)TFA salt

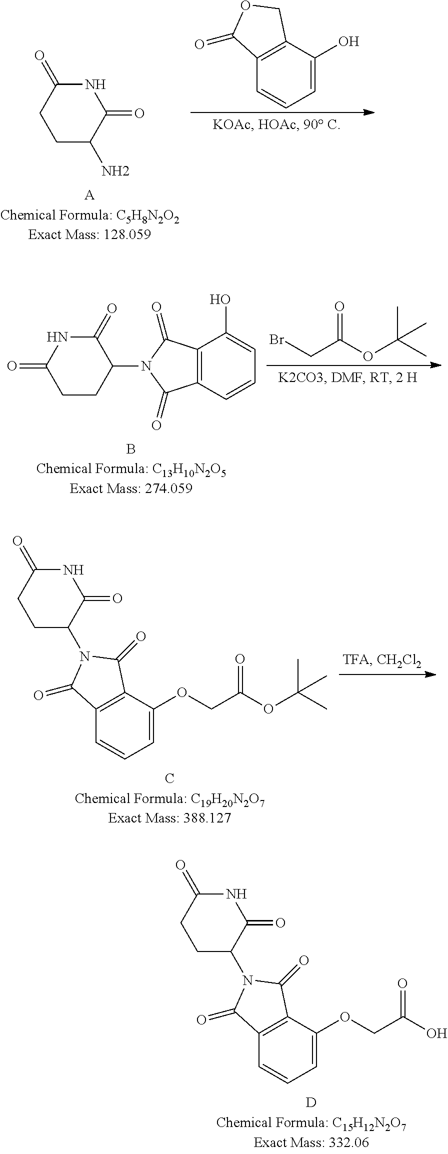

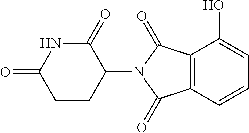 [SMILES: O=C1CCC(N2C(=O)c3cccc(O)c3C2=O)C(=O)N1]

### 2-(2,6-dioxopiperidin-3-yl)-4-hydroxyisoindoline-1,3-dione (B)

A mixture of 3-aminopiperidine-2,6-dione hydrochloride (8.0 g, 48.7 mmol), 4-hydroxyisobenzofuran-1,3-dione (8.0 g, 48.7 mmol) and CH₃COOK (14.3 g, 146.3 mmol) in CH₃COOH (30.0 mL) was stirred at 90° C. for 16 hours. The reaction mixture was poured into H₂O (200 mL), and the resulting solid was collected and dried under vacuum to afford compound B as a gray solid (10.0 g, yield 77%).

LC-MS (ESI) m/z: 275.2 [M+H]⁺.

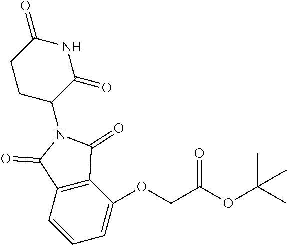 [SMILES: CC(C)(C)OC(=O)COc1cccc2c1C(=O)N(C1CCC(=O)NC1=O)C2=O]

### tert-Butyl 2-((2-(2,6-dioxopiperidin-3-yl)-1,3-dioxoisoindolin-4-yl)oxy)acetate (C)

To a mixture of compound B (7.0 g, 25.5 mmol) and K₂CO₃ (5.27 g, 38.25 mmol) in DMF (30 mL) was added dropped tert-butyl 2-bromoacetate (4.98 g, 25.5 mmol). The resulting mixture was stirred at room temperature for 4 hours, diluted with EtOAc (300 mL), washed with brine (50 mL×2), and dried over anhydrous Na₂SO₄. After filtration and concentration in vacuo, the resulting residue was slurried in a mixture of EtOAc and petroleum ether (100 mL, 1:8 in volume), filtered and dried in vacuo to afford compound C as a white solid (8.7 g, yield 87.8%).

LC-MS (ESI) m/z: 333.0 [M-56+H]⁺.

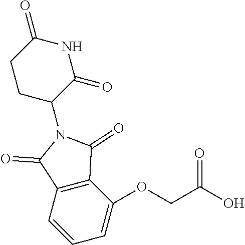 [SMILES: O=C(O)COc1cccc2c1C(=O)N(C1CCC(=O)NC1=O)C2=O]

### 2-((2-(2,6-dioxopiperidin-3-yl)-1,3-dioxoisoindolin-4-yl)oxy)acetic acid (D)

A mixture of compound C (7.7 g, 19.8 mmol) and TFA (20.0 mL) in DCM (20.0 mL) was stirred at room temperature for 2 hours, the mixture was concentrated to afford compound D as a white solid (6.0 g, yield 92%).

LC-MS (ESI) m/z: 333.3 [M+1-1]⁺.

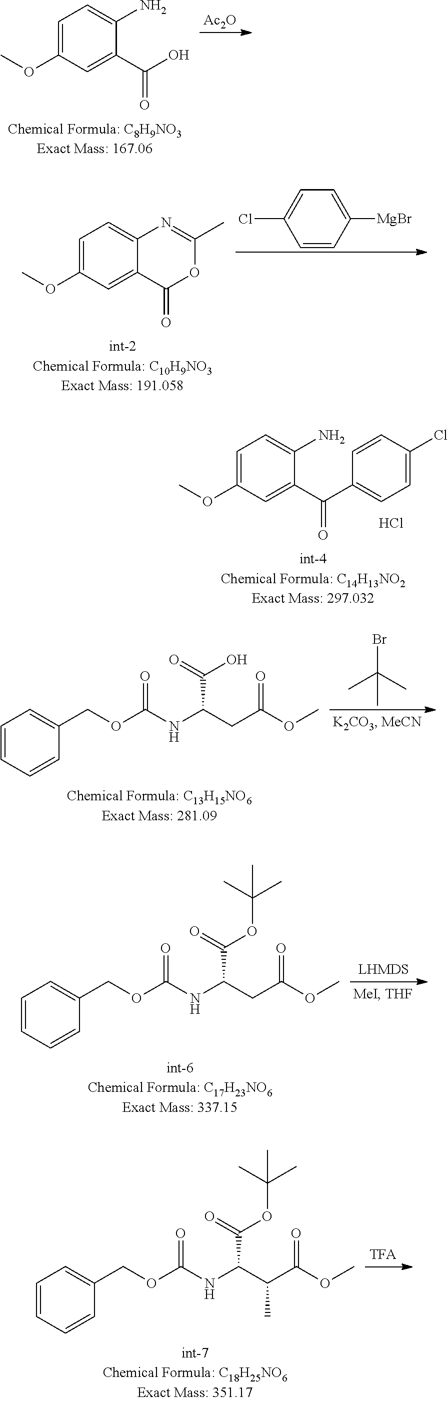

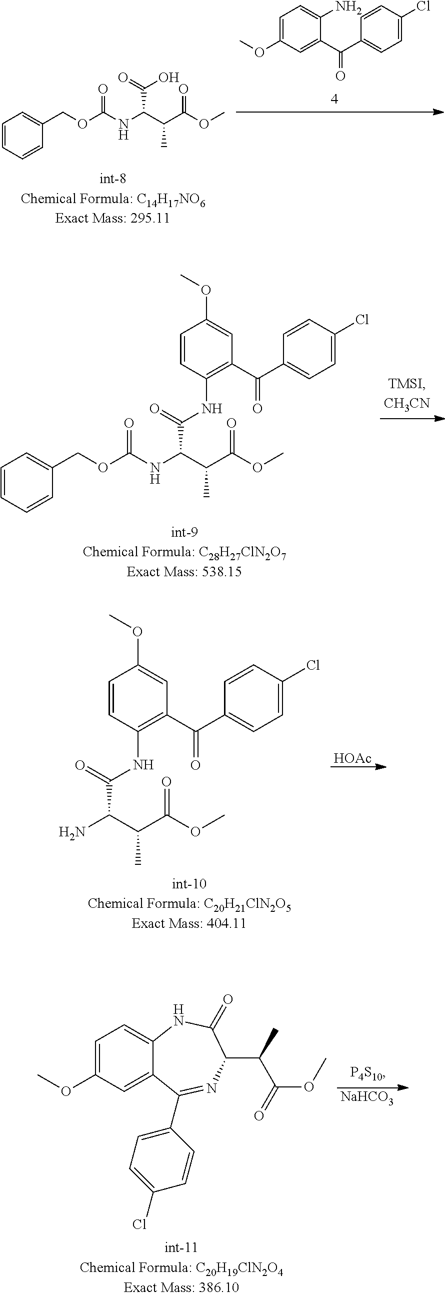

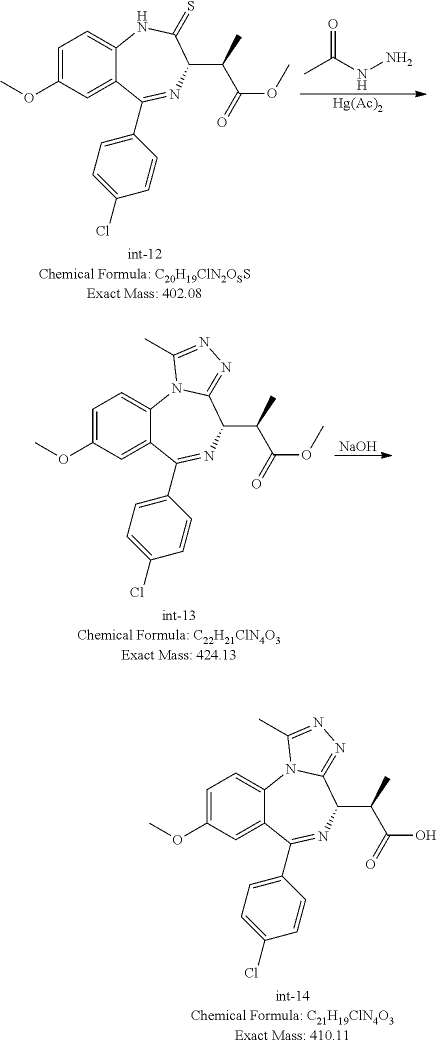

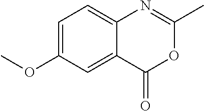

### 6-methoxy-2-methyl-4H-benzo[d][1,3]oxazin-4-one (Int-2)

A solution of 2-amino-5-methoxybenzoic acid (15 g, 89.8 mmol) in acetic anhydride (60 mL) was stirred at 130° C. for 2 hours. The reaction mixture was concentrated in vacuo to afford a crude product, which was washed with n-hexane (40 mL×2) to furnish pure intermediate 2 (int-2) as a white solid (16 g, 93.2% yield).

LC-MS (ESI) m/z: 192[M+H]⁺. ¹H-NMR (CDCl₃, 400 MHz): δ (ppm) 2.46 (s, 3H), 3.90 (dd, J=8.6 Hz, 3H), 7.36 (dd, J=8.8 Hz, 1H), 7.48 (d, J=8.8 Hz, 1H), 7.57 (s, 1H).

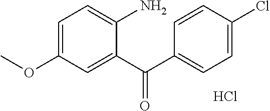 [SMILES: COc1ccc(N)c(C(=O)c2ccc(Cl)cc2)c1 ; Cl]

### (2-amino-5-methoxyphenyl)(4-chlorophenyl)methanone hydrochloride (int-4)

To a solution of int-2 (5 g, 26.2 mmol) in toluene (60 mL) and ether (30 mL) was added dropwise a solution of 4-chlorophenyl magnesium bromide (1 M in THF, 26.2 mL, 26.2 mmol) at 0° C. The reaction mixture was stirred at room temperature for 1 hour and then was quenched with HCl solution (1 N, 25 mL). The mixture was extracted with ethyl acetate (50 mL×3), washed with brine (50 mL), and dried over anhydrous Na₂SO₄. After filtration and concentration in vacuo, the resulting residue was diluted with ethanol (50 mL) and HCl solution (6 N, 20 mL) and then heated to reflux for 2 hours. The mixture was concentrated in vacuo to remove two-thirds of solvents, and the resulting solid was collected, washed with petroleum ether and dried in vacuo to afford int-4 as a white solid (2.1 g, 30.7% yield).

LC-MS (ESI) m/z: 262[M+H]⁺.

¹H-NMR (DMSO-d₆, 400 MHz): δ (ppm) 3.65 (s, 3H), 6.88 (s, 1H), 7.15-7.20 (m, 2H), 7.61 (d, J=8.8 Hz, 2H), 7.70 (d, J=8.4 Hz, 2H).

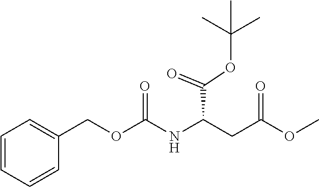 [SMILES: COC(=O)C[C@H](NC(=O)OCc1ccccc1)C(=O)OC(C)(C)C]

### 1-(tert-Butyl) 4-methyl ((benzyloxy)carbonyl)-L-aspartate (int-6)

To a solution of (S)-2-(((benzyloxy)carbonyl)amino)-4-methoxy-4-oxobutanoic acid (15 g, 53.4 mmol) in acetonitrile (180 mL) were added potassium carbonate (47.97 g, 347.1 mmol) and BnEt₃NCl (12.4 g, 54.5 mmol). After stirring at room temperature for 5 hours, 2-bromo-2-methylpropane (73.17 g, 534 mmol) was added to the reaction mixture. The resulting mixture was stirred at 50° C. for 26 hours, cooled down to room temperature, diluted with water (200 mL), and extracted with ethyl acetate (200 mL×3). The combined organic layers were washed with brine (150 mL), dried over anhydrous Na₂SO₄, and concentrated in vacuo. The resulting crude product was purified by flash column chromatography on silica gel (ethyl acetate in petroleum ether, 20% v/v) to afford int-6 as a colorless oil (12 g, yield 66.5%).

LC-MS (ESI) m/z: 238[M-Boc+H]⁺.

¹H-NMR (CDCl₃, 400 MHz): δ (ppm) 1.45 (s, 9H), 2.82 (dd, J=16.6 Hz, 1H), 2.98 (dd, J=16.6 Hz, 1H), 3.66 (s, 3H), 4.50-4.54 (m, 1H), 5.12 (s, 2H), 5.73 (d, J=8.0 Hz, 1H), 7.27-7.36 (m, 5H).

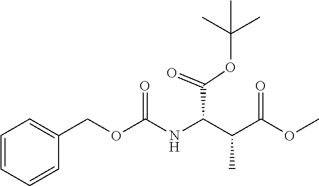 [SMILES: COC(=O)[C@H](C)[C@H](NC(=O)OCc1ccccc1)C(=O)OC(C)(C)C]

### 1-(tert-Butyl) 4-methyl (2S,3R)-2-(((benzyloxy)carbonyl)amino)-3-methylsuccinate (int-7)

A solution of int-6 (8.8 g, 26.1 mmol) in anhydrous THF (105 mL) was cooled to −78° C., and then LiHMDS solution (1 M in THF, 54.8 mL, 54.8 mmol) was added dropwise. The mixture was stirred at −78° C. under nitrogen atmosphere for 45 minutes, and iodomethane (11.1 g, 78.3 mmol) was added dropwise. The resulting mixture was stirred at −78° C. for 3 hours and quenched with saturated NH₄Cl solution (50 mL). The resulting mixture was extracted with ethyl acetate (100 mL×3). The combined organic layers were dried over anhydrous Na₂SO₄ and concentrated in vacuo. The resulting crude product was purified by flash column chromatography on silica gel (ethyl acetate in petroleum ether, 20% v/v) to afford int-7 as a colorless oil (8.8 g, yield 96%).

LC-MS (ESI) m/z: 252[M-Boc+H]⁺.

¹H-NMR (CDCl₃, 400 MHz): δ (ppm) 1.21 (d, J=7.6 Hz 3H), 1.46 (s, 9H), 3.17-3.23 (m, 1H), 3.69 (s, 3H), 4.57 (dd, J=9.0 Hz, 1H), 5.13 (s, 2H), 5.64 (d, J=8.4 Hz, 1H), 7.27-7.37 (m, 5H).

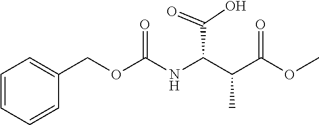 [SMILES: COC(=O)[C@H](C)[C@H](NC(=O)OCc1ccccc1)C(=O)O]

### (2S,3R)-2-(((Benzyloxy)carbonyl)amino)-4-methoxy-3-methyl-4-oxobutanoic acid (Int-8)

To a solution of int-7 (8.8 g, 25.07 mmol) in dichloromethane (120 mL) was added TFA (40 mL). The reaction mixture was stirred at room temperature overnight and concentrated in vacuo to afford crude int-8 as oil (9 g, crude), which was used directly in the next step without further purification.

LC-MS (ESI) m/z: 296[M+H]⁺.

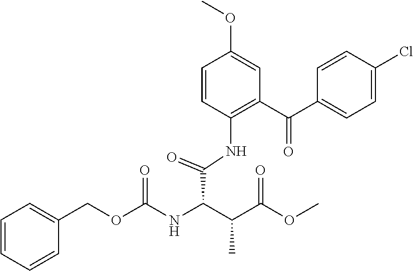 [SMILES: COC(=O)[C@H](C)[C@H](NC(=O)OCc1ccccc1)C(=O)Nc1ccc(OC)cc1C(=O)c1ccc(Cl)cc1]

### Methyl (2R,3S)-3-(((benzyloxy)carbonyl)amino)-4-((2-(4-chlorobenzoyl)-4-methoxyphenyl)amino)-2-methyl-4-oxobutanoate (int-9)

To a solution of int-8 (9 g crude, 25.07 mmol) in dichloromethane (180 mL) were added HATU (19.06 g, 50.14 mmol), DIPEA (12.96 g, 100.28 mmol) and int-4 (7.44 g, 25.07 mmol) at 0° C. The reaction mixture was stirred at room temperature for 2 hours, diluted with dichloromethane (120 mL), washed with brine (100 mL×3), dried over anhydrous Na₂SO₄, filtered and concentrated in vacuo. The resulting residue was purified by flash column chromatography on silica gel (ethyl acetate in petroleum ether, 30% v/v) to afford int-9 as a light yellow solid (10 g, yield 74.1%).

LC-MS (ESI) m/z: 539[M+H]⁺.

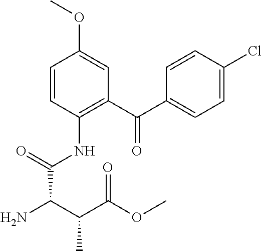 [SMILES: COC(=O)[C@H](C)[C@H](N)C(=O)Nc1ccc(OC)cc1C(=O)c1ccc(Cl)cc1]

### Methyl (2R,3S)-3-amino-4-((2-(4-chlorobenzoyl)-4-methoxyphenyl)amino)-2-methyl oxobutanoate (int-10)

To a solution of int-9 (10 g, 18.6 mmol) in acetonitrile (200 mL) was added iodotrimethylsilane (8.23 g, 41.1 mmol) at room temperature. The reaction mixture was stirred at room temperature for 2 hours and then was diluted with ethyl acetate (100 mL). The resulting mixture was washed with saturated NaHSO₃ solution (100 mL×2) and brine (100 mL), dried over Na₂SO₄, filtered and concentrated in vacuo to afford crude int-10 as oil (12 g, crude), which was used directly in the next step without further purification.

LC-MS (ESI) m/z: 405[M+H]⁺.

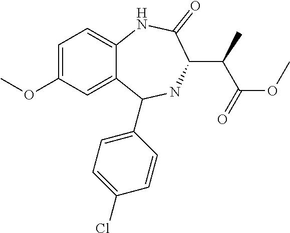

### Methyl (R)-2-(S)-5-(4-chlorophenyl)-7-methoxy-2-oxo-2,3-dihydro-1H-benzo[e][1,4]diazepin-3-yl)propanoate (int-11)

To a solution of int-10 (10 g, 15.5 mmol) in 1,2-dichloroethane (116 mL) was added acetic acid (10.5 g, 173 mmol) at room temperature. The reaction mixture was stirred at 60° C. for 2 hours and then concentrated in vacuo. The resulting residue was dissolved in dichloromethane (150 mL), washed with brine (50 mL×2), dried over anhydrous Na₂SO₄ and concentrated in vacuo. The resulting crude product was purified by flash column chromatography on silica gel (ethyl acetate in petroleum ether, 50% v/v) to afford int-11 as a light solid (3.5 g, yield 36.6%).

LC-MS (ESI) m/z: 387[M+H]⁺.

¹H-NMR (CDCl₃, 400 MHz): δ (ppm) 1.44 (d, J=7.2 Hz, 3H), 3.53-3.58 (m, 1H), 3.74 (s, 3H), 3.76 (s, 3H), 3.84 (d, J=10.4 Hz, 1H), 6.74 (s, 1H), 7.08-7.11 (m, 2H), 7.35 (d, J=8.4 Hz, 2H), 7.52 (d, J=8.8 Hz, 2H), 9.23 (s, 1H).

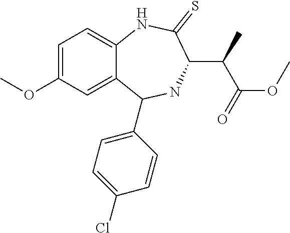

### Methyl (R)-2-(S)-5-(4-chlorophenyl)-7-methoxy-2-thioxo-2,3-dihydro-1H-benzo[e][1,4]diazepin-3-yl)propanoate (int-12)

A mixture of P₄S₁₀ (2.07 g, 4.66 mmol) and NaHCO₃ (0.786 g, 9.36 mmol) in 1,2-dichloroethane (40 mL) was stirred at room temperature for 2 hours, and then int-11 (1 g, 2.6 mmol) was added. The resulting mixture was stirred at 70° C. for 2 hours and then was concentrated in vacuo. The resulting residue was partitioned between dichloromethane (50 mL) and water (50 mL). The organic layer was separated, washed with brine (50 mL), dried over anhydrous Na₂SO₄ and concentrated in vacuo. The resulting crude product was purified by flash column chromatography on silica gel (ethyl acetate in petroleum ether, 30% v/v) to afford int-12 as a light yellow solid (0.8 g, yield 76.8%).

LC-MS (ESI) m/z: 403[M+H]⁺.

¹H-NMR (CDCl₃, 400 MHz): δ (ppm) 1.38 (d, J=6.8 Hz, 3H), 3.76 (s, 3H), 3.80 (s, 3H), 3.90 (d, J=9.6 Hz, 1H), 4.05-4.15 (m, 1H), 6.78 (s, 1H), 7.08-7.14 (m, 2H), 7.35 (d, J=8.8 Hz, 2H), 7.52 (d, J=8.8 Hz, 2H), 9.23 (s, 1H).

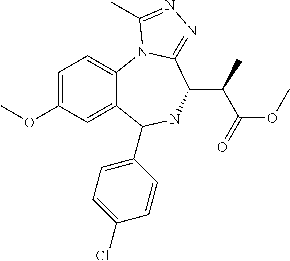

### Methyl (R)-2-((S)-6-(4-chlorophenyl)-8-methoxy-1-methyl-4H-benzo[f][1,2,4]triazolo[4,3-a][1,4]diazepin-4-yl)propanoate (int-13)

A suspension of int-12 (2.5 g, 6.21 mmol) and acetyl hydrazide (1.38 g, 18.6 mmol) in THF (37.5 mL) and acetic acid (25 mL) was allowed to cool to 0° C. Mercury acetate (2.97 g, 9.32 mmol) was added portionwise to the mixture in order to maintain the reaction temperature below 5° C. The reaction mixture was stirred at 0° C. for 2 hours and then was stirred at room temperature overnight. The resulting mixture was partitioned between ethyl acetate (50 mL) and then was saturated NaHCO₃ solution (50 mL). The organic layer was separated, and the aqueous layer was re-extracted with ethyl acetate (50 mL×3). The combined organic layers were dried over Na₂SO₄, filtered and concentrated in vacuo. The resulting residue was purified by flash column chromatography on silica gel (methanol in dichloromethane, 10% v/v) to give int-13 as a light yellow solid (1.5 g, yield 56.9%).

LC-MS (ESI) m/z: 425[M+H]⁺.

¹H-NMR (CDCl₃, 400 MHz): δ (ppm) 1.49 (d, J=6.8 Hz, 3H), 2.61 (s, 3H), 3.82 (s, 3H), 3.83 (s, 3H), 4.06-4.15 (m, 1H), 4.25 (d, J=10.8 Hz, 1H), 6.88 (s, 1H), 7.23 (dd, J=9.0 Hz, 1H), 7.32 (d, J=6.8 Hz, 2H), 7.42 (q, J=8.4 Hz, 3H).

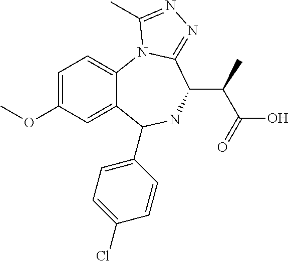

(R)-2-(S)-6-(4-Chlorophenyl)-8-methoxy-1-methyl-4H-benzo[f][1,2,4]triazolo[4,3-a][1,4]diazepin-4-yl)propanoic acid (int-14)

To a solution of int-13 (0.2 g, 0.471 mmol) in THF (3 mL) was added NaOH solution (1 M, 0.943 mL, 0.943 mmol). The reaction mixture was stirred at room temperature for 48 hours. The mixture was adjusted to pH 3˜4 with 1 M HCl, and the resulting mixture was extracted with ethyl acetate (10 mL×3). The combined organic layers were dried over anhydrous Na₂SO₄, filtered and concentrated in vacuo to afford crude int-14 as solid (0.15 g, yield 78%), which was used directly in the next step without further purification.

LC-MS (ESI) m/z: 411[M+H]⁺.

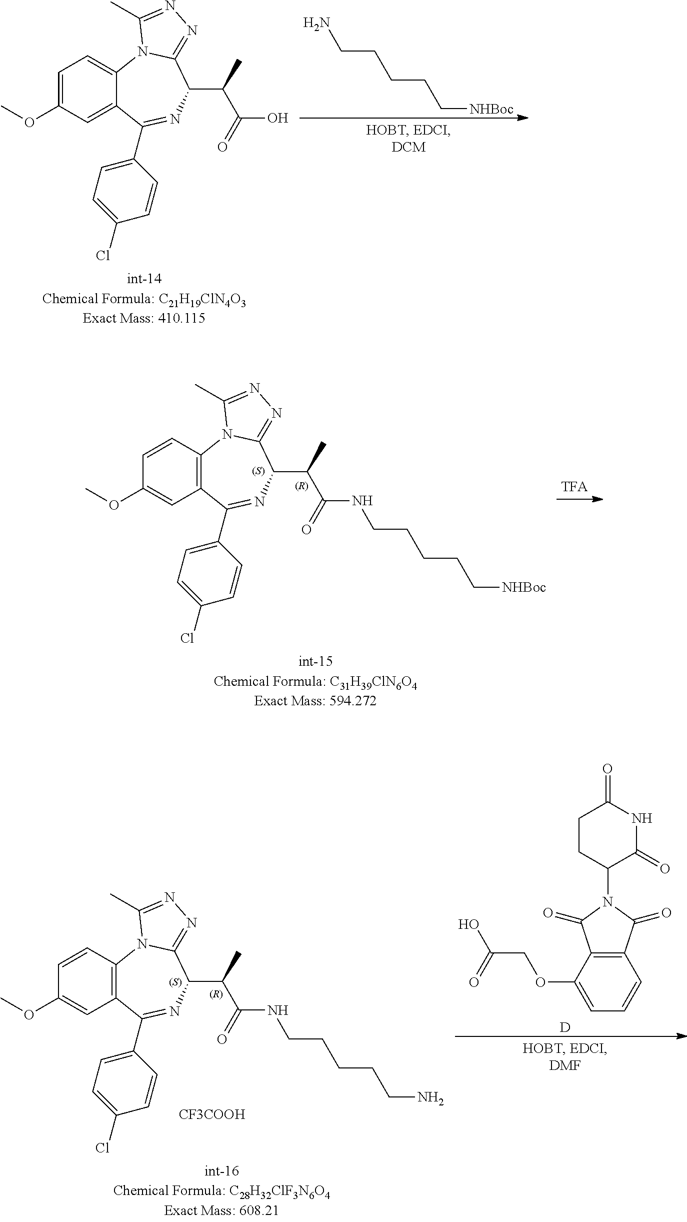

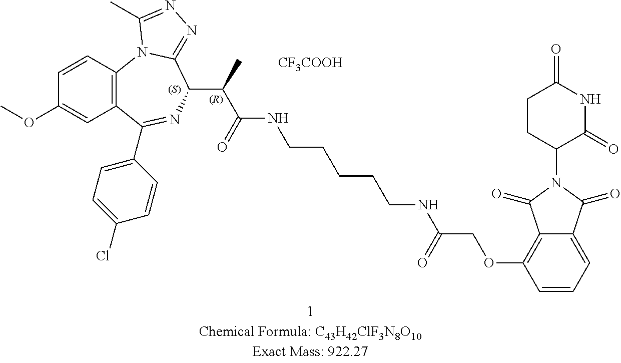

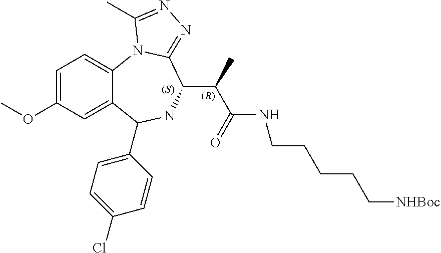

### tert-Butyl (5-((2R)-2-((4S)-6-(4-chlorophenyl)-8-methoxy-1-methyl-4H-benzo[f][1,2,4]triazolo[4,3-a][1,4]diazepin-4-yl)propanamido)pentyl) carbamate (int-15)

To a solution of int-14 (0.1 g, 0.244 mmol) and N-Boc-1,5-diaminopentane (99 mg, 0.488 mmol) in dichloromethane (5 mL) were added HOBt (39 mg, 0.293 mmol) and 1-ethyl-3-(3′-dimethylaminopropyl)carbodiimide (EDC)·HCl (56.2 mg, 0.293 mmol) at 0° C. The reaction mixture was stirred at room temperature under nitrogen for 2 hours, diluted with dichloromethane (30 mL) and washed with brine (10 mL×2). The combined organic layers were dried over anhydrous Na₂SO₄, filtered and concentrated in vacuo. The resulting crude product was purified by flash column chromatography on silica gel (methanol in dichloromethane, 15% v/v) to afford int-15 as a white solid (0.12 g, 82.7% yield).

LC-MS (ESI) m/z: 595[M+H]⁺.

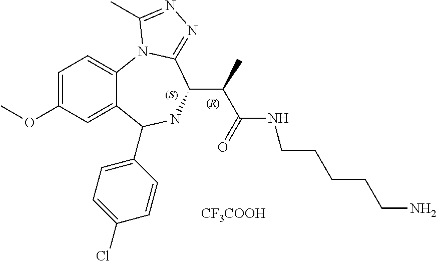

### (2R)—N-(5-aminopentyl)-2-((4S)-6-(4-chlorophenyl)-8-methoxy-1-methyl-4H-benzo[f][1,2,4]triazolo[4,3-a][1,4]diazepin-4-yl)propanamide TFA salt (Int-16)

To a solution of int-15 (0.15 g, 0.252 mmol) in dichloromethane (3 mL) was added TFA (1 mL). The reaction mixture was stirred at room temperature for 30 minutes and concentrated in vacuo. The resulting residue was diluted with dichloromethane (30 mL), washed with saturated NaHCO₃ solution (10 mL×3) and brine (10 mL). The aqueous layers were combined and re-extracted with 10% methanol in dichloromethane (30 mL×2). The combined organic layers were dried over anhydrous Na₂SO₄, filtered and concentrated in vacuo to afford crude int-16 as oil (0.11 g, crude), which was used directly in the next step without further purification.

LC-MS (ESI) m/z: 495[M+H]⁺.

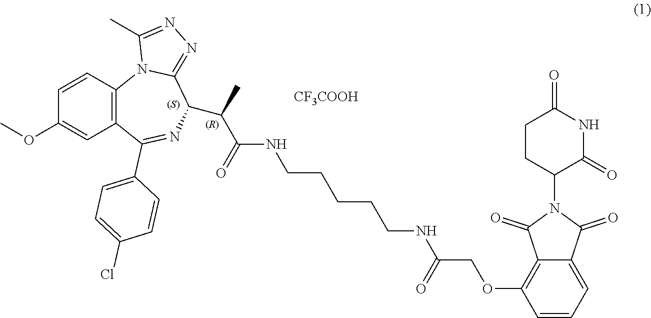

To a solution of int-16 (90 mg crude, 0.148 mmol) and compound D (72.6 mg, 0.218 mmol) in anhydrous DMF (4.5 mL) were added HOBt (29.5 mg, 0.218 mmol) and EDC·HCl (41.8 mg, 0.218 mmol) at 0° C. The reaction mixture was stirred at room temperature under nitrogen for 2 hours, diluted with water (30 mL) and extracted with ethyl acetate (30 mL×3). The combined organic layers were washed with brine (30 mL), dried over anhydrous Na₂SO₄, filtered and concentrated in vacuo, the residue was purified by preparative HPLC (MeCN—H₂O-TFA, 35%-70% gradient) to afford compound 1 as a white solid (102 mg, 74.7% yield).

LC-MS (ESI) m/z: 809[M+H]⁺.

¹H NMR (500 MHz, DMSO-d₆) δ 11.10 (s, 1H), 8.31-8.19 (m, 1H), 7.97 (t, J=5.7 Hz, 1H), 7.81 (d, J=8.9 Hz, 1H), 7.81 (t, J=8.4 Hz, 1H), 7.48 (d, 1H), 7.41-7.36 (m, 2H), 6.87 (dd, J=2.9, 1.0 Hz, 1H), 5.12 (ddd, J=12.8, 5.4, 1.7 Hz, 1H), 4.77 (s, 2H), 4.11 (d, J=10.8 Hz, 1H), 3.79 (s, 3H), 3.65 (dq, J=10.7, 6.7 Hz, 1H), 3.19 (dq, J=17.6, 6.7 Hz, 3H), 3.07 (dq, J=12.8, 6.6 Hz, 1H), 2.88 (ddd, J=17.4, 14.1, 5.5 Hz, 1H), 2.64-2.46 (m, 2H), 2.54 (s, 3H), 2.04 (dt, J=12.9, 3.1 Hz, 1H), 1.59-1.45 (m, 4H), 1.42-1.30 (m, 2H), 1.21 (d, J=6.7 Hz, 3H).

¹³C NMR (126 MHz, DMSO) δ 173.62, 172.75, 169.86, 166.72, 166.66, 165.51, 164.73, 158.32, 158.03, 157.65, 155.05, 154.82, 150.78, 137.00, 136.92, 135.45, 133.02, 131.00, 129.27, 128.17, 126.00, 125.84, 120.40, 117.92, 116.83, 116.04, 114.89, 67.67, 59.26, 55.85, 48.80, 41.94, 38.38, 38.26, 30.93, 29.06, 28.72, 23.72, 21.99, 15.57, 11.40.

### Example 3: Synthesis of (2R)-2-((4S)-6-(4-chlorophenyl)-8-methoxy-1-methyl-4H-benzo[f][1,2,4]triazolo[4,3-a][1,4]diazepin-4-yl)-N-(7-(2-((2-(2,6-dioxopiperidin-3-yl)-1,3-dioxoisoindolin-4-yl)oxy)acetamido)heptyl)propanamide (2) TFA salt

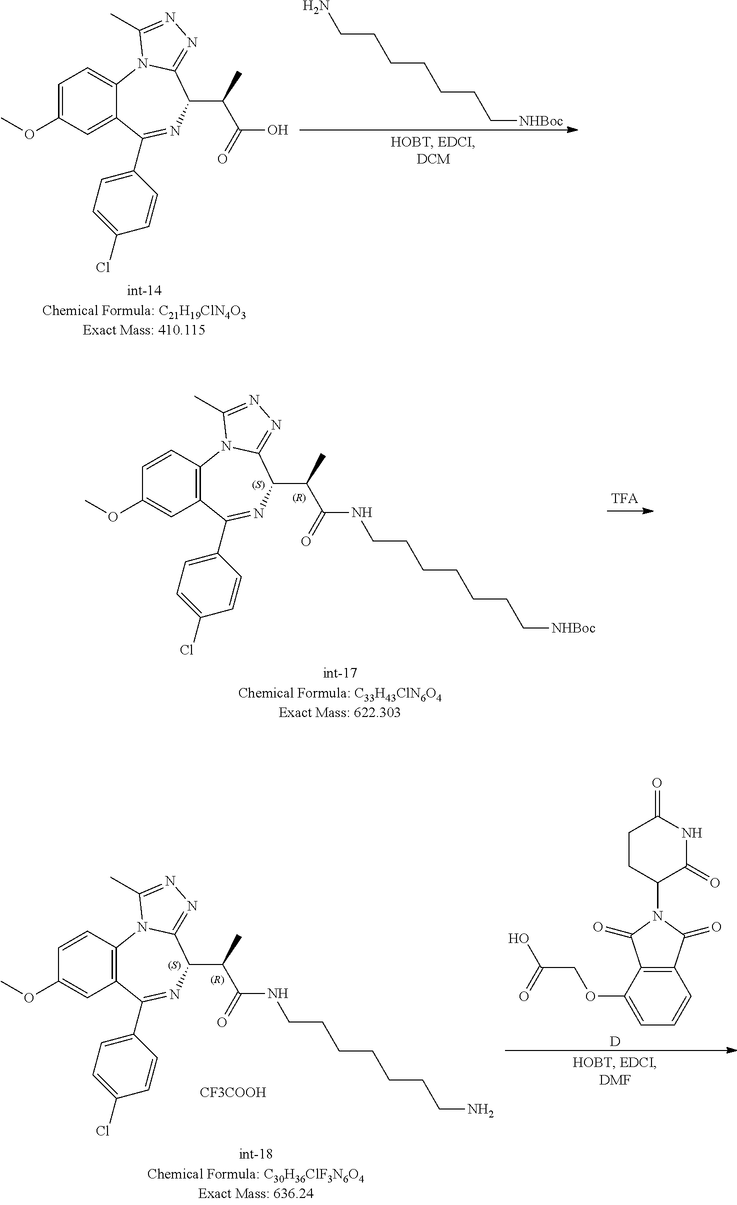

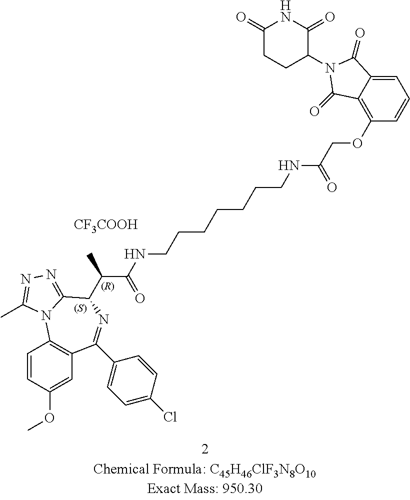

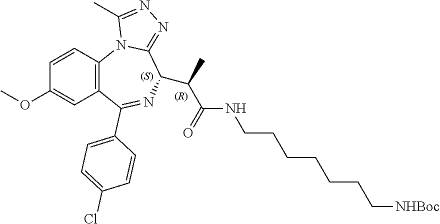

### tert-Butyl (7-((2R)-2-((4S)-6-(4-chlorophenyl)-8-methoxy-1-methyl-4H-benzo[f][1,2,4]triazolo[4,3-α][1,4]diazepin-4-yl)propanamido)heptyl) carbamate (int-17)

To a solution of int-14 (60 mg, 0.146 mmol) and 1-Boc-1,7-diaminoheptane (40.45 mg, 0.175 mmol) in dichloromethane (2 mL) were added HOBt (23.6 mg, 0.175 mmol) and EDC·HCl (33.5 mg, 0.175 mmol) at 0° C. The reaction mixture was stirred at room temperature under nitrogen for 2 hours, diluted with dichloromethane (30 mL) and washed with brine (10 mL×2). The combined organic layers were dried over anhydrous Na₂SO₄, filtered and concentrated in vacuo. The resulting residue was purified by flash column chromatography on silica gel (methanol in dichloromethane, 15% v/v) to afford int-17 as a white solid (65 mg, 71.4% yield).

LC-MS (ESI) m/z: 623[M+H]⁺.

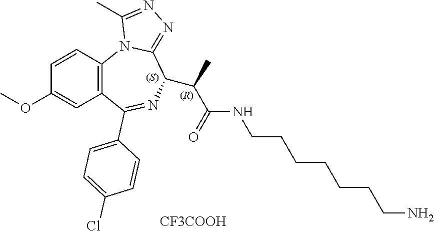

### (2R)—N-(7-Aminoheptyl)-2-44S)-6-(4-chlorophenyl)-8-methoxy-1-methyl-4H-benzo[f][1,2,4]triazolo[4,3-α][1,4]diazepin-4-yl)propanamide TFA salt (int-18)

To a solution of int-17 (65 mg, 0.104 mmol) in dichloromethane (1.5 mL) was added TFA (0.5 mL). The reaction mixture was stirred at room temperature for 30 minutes and concentrated in vacuo. The resulting residue was diluted with dichloromethane (30 mL), washed with saturated NaHCO₃ solution (10 mL×3) and brine (10 mL). The aqueous layers were combined and re-extracted with 10% methanol in dichloromethane (30 mL×2). The combined organic layers were dried over anhydrous Na₂SO₄, filtered and concentrated in vacuo to afford crude int-18 as oil (65 mg, crude), which was used directly for the next step without further purification.

LC-MS (ESI) m/z: 523[M+H]⁺.

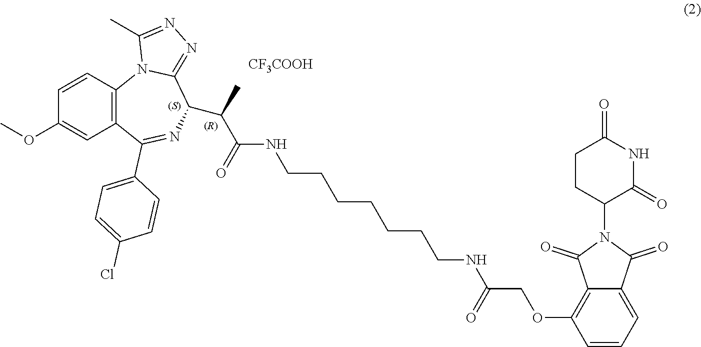

To a solution of int-18 (65 mg crude, 0.102 mmol) and compound D (47 mg, 0.142 mmol) in anhydrous DMF (2 mL) were added HOBt (19.2 mg, 0.142 mmol) and EDC·HCl (27.2 mg, 0.142 mmol) at 0° C. The reaction mixture was stirred at room temperature under nitrogen for 2 hours, diluted with water (10 mL) and extracted with ethyl acetate (30 mL×3). The combined organic layers were washed with brine (30 mL), dried over anhydrous Na₂SO₄, filtered and concentrated in vacuo. The resulting residue was purified by preparative HPLC (MeCN—H₂O-TFA, 35%-70% gradient) to afford compound 2 as a white solid (46 mg, 47.4% yield).

LC-MS (ESI) m/z: 837[M+H]⁺.

¹H NMR (500 MHz, DMSO-d₆) δ 11.11 (s, 1H), 8.24 (t, J=5.6 Hz, 1H), 7.93 (t, J=5.8 Hz, 1H), 7.81 (d, J=9.1 Hz, 1H), 7.80 (t, J=8.5 Hz, 1H), 7.51-7.43 (m, 5H), 7.39 (dd, J=8.8, 3.3 Hz, 2H), 6.86 (d, J=2.9 Hz, 1H), 5.11 (dd, J=12.7, 5.4 Hz, 1H), 4.76 (s, 2H), 4.11 (d, J=10.7 Hz, 1H), 3.79 (s, 3H), 3.65 (dq, J=10.8, 6.7 Hz, 1H), 3.25-3.01 (m, 4H), 2.89 (ddd, J=16.7, 13.7, 5.4 Hz, 1H), 2.63-2.51 (m, 2H), 2.54 (s, 3H), 2.04 (ddd, J=13.1, 5.8, 3.5 Hz, 1H), 1.56-1.38 (m, 4H), 1.31 (d, J=5.2 Hz, 6H), 1.21 (d, J=6.7 Hz, 3H).

¹³C NMR (126 MHz, DMSO) δ 174.06, 173.23, 170.33, 167.20, 167.08, 165.98, 165.15, 158.78, 158.49, 158.10, 155.53, 155.30, 151.24, 137.50, 137.38, 135.91, 133.50, 131.50, 129.72, 128.63, 126.52, 126.33, 120.85, 118.35, 117.29, 116.51, 115.37, 68.12, 59.75, 56.32, 49.28, 42.44, 38.90, 38.78, 31.41, 29.84, 29.47, 28.97, 26.93, 26.80, 22.47, 16.05, 11.89.

### Example 4: Synthesis of (2R)-2-((4S)-6-(4-chlorophenyl)-8-methoxy-1-methyl-4H-benzo[f][1,2,4]triazolo[4,3-α][1,4]diazepin-4-yl)-N-(9-(2-((2-(2,6-dioxopiperidin-3-yl)-1,3-dioxoisoindolin-4-yl)oxy)acetamido)nonyl)propanamide (3) TFA salt

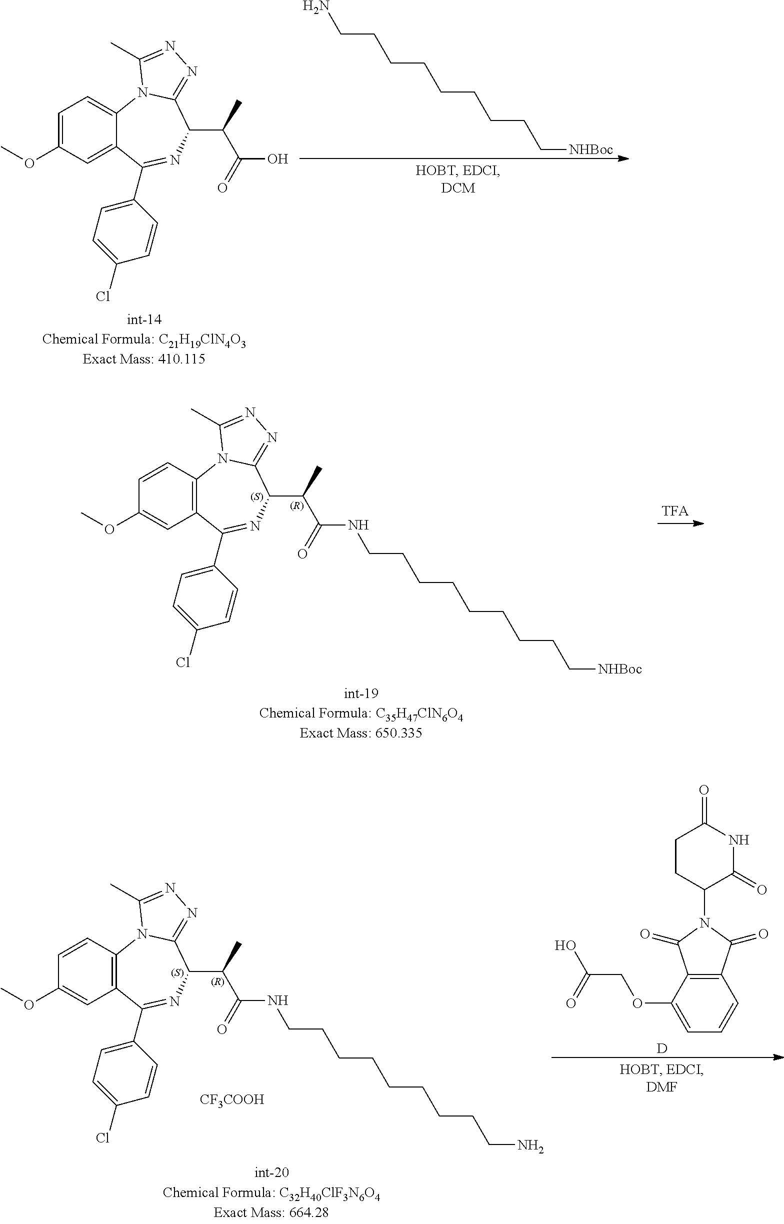

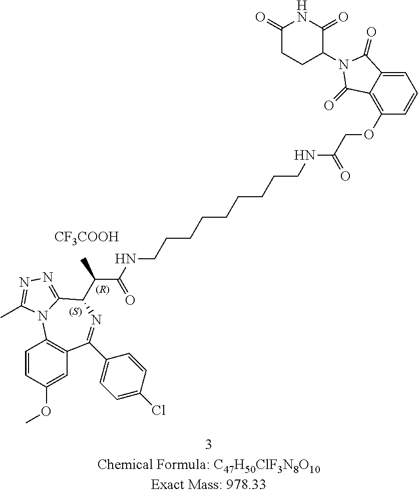

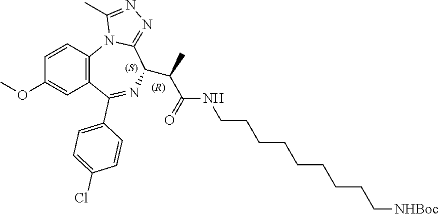

### tert-Butyl (9-((2R)-2-((4S)-6-(4-chlorophenyl)-8-methoxy-1-methyl-4H-benzo[f][1,2,4]triazolo[4,3-α][1,4]diazepin-4-yl)propanamido)nonyl) carbamate (int-19)

To a solution of int-14 (60 mg, 0.146 mmol) and 1-Boc-1,9-diaminononane (45.2 mg, 0.175 mmol) in dichloromethane (2 mL) were added HOBt (23.6 mg, 0.175 mmol) and EDC·HCL (33.5 mg, 0.175 mmol) at 0° C. The reaction mixture was stirred at room temperature under nitrogen for 2 hours, diluted with dichloromethane (30 mL) and washed with brine (10 mL×2). The combined organic layers were dried over anhydrous Na₂SO₄, filtered and concentrated in vacuo. The resulting crude product was purified by flash column chromatography on silica gel (methanol in dichloromethane, 15% v/v) to afford int-19 as a white solid (70 mg, 73.5% yield).

LC-MS (ESI) m/z: 651[M+H]⁺.

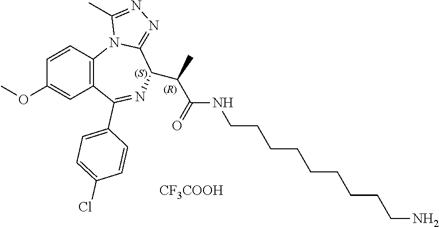

### (2R)—N-(9-Aminononyl)-2-((4S)-6-(4-chlorophenyl)-8-methoxy-1-methyl-4H-benzo[f][1,2,4]triazolo[4,3-α][1,4]diazepin-4-yl)propanamide TFA salt (int-20)

To a solution of int-19 (70 mg, 0.108 mmol) in dichloromethane (1.5 mL) was added TFA (0.5 mL). The reaction mixture was stirred at room temperature for 30 minutes and concentrated in vacuo. The resulting residue was diluted with dichloromethane (30 mL), washed with saturated NaHCO₃ solution (10 mL×3) and brine (10 mL). The aqueous layers were combined and re-extracted with 10% methanol in dichloromethane (30 mL×2). The combined organic layers were dried over anhydrous Na₂SO₄, filtered and concentrated in vacuo to afford crude int-20 as oil (65 mg, crude), which was used directly for the next step without further purification.

LC-MS (ESI) m/z: 551[M+H]⁺.

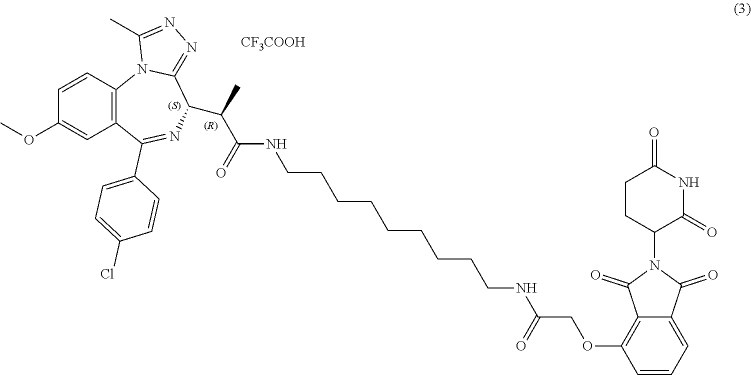

To a solution of int-20 (65 mg crude, 0.978 mmol) and compound D (47 mg, 0.142 mmol) in anhydrous DMF (2 mL) were added HOBt (19.2 mg, 0.142 mmol) and EDC·HCl (27.2 mg, 0.142 mmol) at 0° C. The reaction mixture was stirred at room temperature under nitrogen for 2 hours, diluted with water (10 mL) and extracted with ethyl acetate (20 mL×3). The combined organic layers were washed with brine (20 mL), dried over anhydrous Na₂SO₄, filtered and concentrated in vacuo. The resulting residue was purified by preparative HPLC (MeCN—H₂O-TFA, 35%-70% gradient) to afford compound 3 as a white solid (43 mg, 44.9% yield).

LC-MS (ESI) m/z: 865[M+H]⁺.

¹H-NMR (DMSO-d₆, 400 MHz) δ (ppm) 11.12 (s, 1H), 8.24 (brs, 1H), 7.93 (brs, 1H), 7.80 (dd, J=12.5, 6.8 Hz, 2H), 7.50-7.35 (m, 7H), 6.84 (d, J=2.4 Hz, 1H), 5.13-5.05 (m, 1H), 4.75 (s, 2H), 4.10 (d, J=10.9 Hz, 1H), 3.78 (s, 3H), 3.27-3.02 (m, 4H), 2.95-2.81 (m, 1H), 2.60-2.49 (m, 6H), 2.08-1.98 (m, 1H), 1.52-1.44 (m, 4H), 1.28-1.15 (m, 14H).

### Example 5: Synthesis of (2R)-2-((4S)-6-(4-chlorophenyl)-8-methoxy-1-methyl-4H-benzo[f][1,2,4]triazolo[4,3-α][1,4]diazepin-4-yl)-N-(2-(2-(2-((2-(2,6-dioxopiperidin-3-yl)-1,3-dioxoisoindolin-4-yl)oxy)acetamido)ethoxy)ethyl)propanamide (4) TFA salt

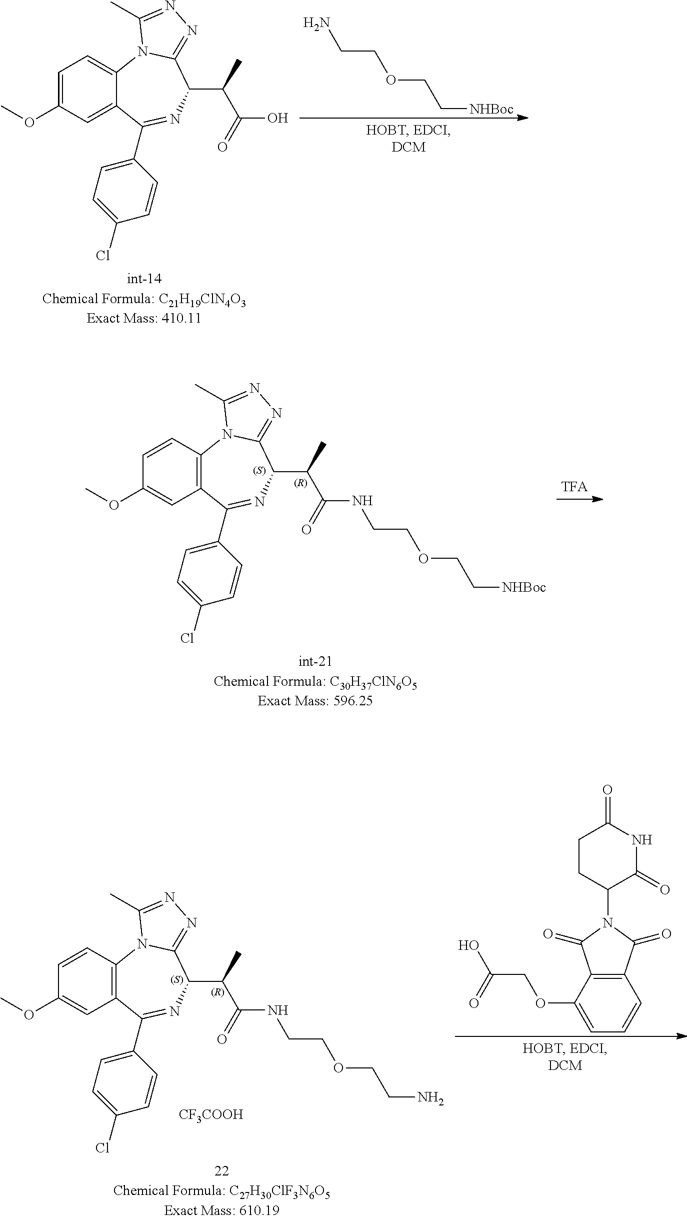

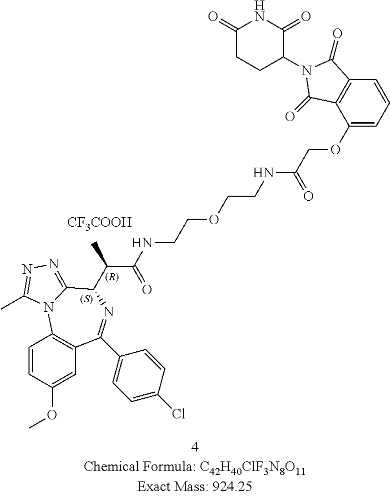

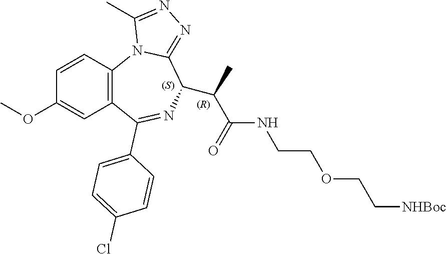

### (2-(2-((2R)-2-44S)-6-(4-Chlorophenyl)-8-methoxy-1-methyl-4H-benzo[f][1,2,4]triazolo[4,3-α][1,4]diazepin-4-yl)propanamido)ethoxy)ethyl) carbamate (int-21)

To a solution of int-14 (0.1 g, 0.244 mmol) and N-Boc-2-aminoethyl 2-amino ethyl ether (99.6 mg, 0.488 mmol) in dichloromethane (5 mL) were added HOBt (39 mg, 0.293 mmol) and EDC·HCL (56.2 mg, 0.293 mmol) at 0° C. The reaction mixture was stirred at room temperature under nitrogen for 2 hours, diluted with dichloromethane (50 mL), washed with brine (20 mL×2). The combined organic layers were dried over anhydrous Na₂SO₄, filtered and concentrated in vacuo. The resulting crude product was purified by flash column chromatography on silica gel (methanol in dichloromethane, 15% v/v) to yield int-21 as a white solid (0.125 g, 86% yield).

LC-MS (ESI) m/z: 597[M+H]⁺.

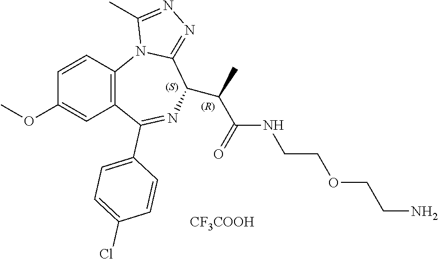

### (2R)—N-(2-(2-Aminoethoxy)ethyl)-2-((4S)-6-(4-chlorophenyl)-8-methoxy-1-methyl-4H-benzo[f][1,2,4]triazolo[4,3-a][1,4]diazepin-4-yl)propanamide TFA salt (int-22)

To a solution of int-21 (0.13 g, 0.218 mmol) in dichloromethane (3 mL) was added TFA (1 mL). The reaction mixture was stirred at room temperature for 30 minutes and concentrated in vacuo. The resulting residue was diluted with dichloromethane (30 mL), washed with saturated NaHCO₃ solution (10 mL×3) and brine (10 mL). The aqueous layers were combined and re-extracted with 10% methanol in dichloromethane (30 mL×2). The combined organic layers were dried over anhydrous Na₂SO₄, filtered and concentrated in vacuo to afford crude int-22 as an oil (0.12 g, 90.2% yield (crude)), which was used directly in the next step without further purification.

LC-MS (ESI) m/z: 497[M+H]⁺.

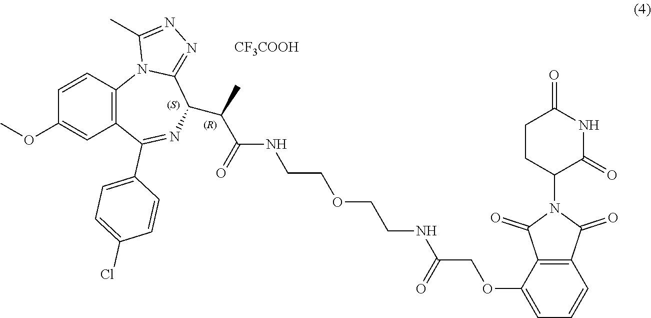

To a solution of int-22 (0.12 g crude, 0.196 mmol) and compound D (96.4 mg, 0.290 mmol) in anhydrous DMF (6 mL) were added HOBt (39.2 mg, 0.290 mmol) and EDC·HCL (55.6 mg, 0.290 mmol) at 0° C. The reaction mixture was stirred at room temperature under nitrogen for 2 hours, diluted with water (30 mL) and extracted with ethyl acetate (30 mL×3). The combined organic layers were washed with brine (30 mL), dried over anhydrous Na₂SO₄, filtered and concentrated in vacuo. The resulting residue was purified by preparative HPLC (MeCN—H₂O-TFA, 35%-70% gradient) to afford compound 4 as a white solid (78 mg, 42.9% yield).

LC-MS (ESI) m/z: 811[M+H]⁺.

¹H NMR (500 MHz, DMSO-d₆) δ 11.10 (s, 1H), 8.34 (q, J=5.3 Hz, 1H), 8.05 (t, J=5.6 Hz, 1H), 7.81 (d, J=9.0 Hz, 1H), 7.78 (dd, J=8.5, 7.3 Hz, 1H), 7.49-7.42 (m, 5H), 7.39 (ddd, J=9.0, 2.9, 0.9 Hz, 1H), 7.35 (dd, J=8.6, 1.9 Hz, 1H), 6.86 (d, J=2.9 Hz, 1H), 5.12 (dt, J=13.1, 4.9 Hz, 1H), 4.79 (s, 2H), 4.11 (dd, J=10.8, 1.1 Hz, 1H), 3.79 (s, 3H), 3.69 (dq, J=10.7, 6.7 Hz, 1H), 3.59-3.48 (m, 4H), 3.46-3.22 (m, 4H), 3.10 (qd, J=7.3, 4.8 Hz, 1H), 2.88 (ddd, J=18.0, 14.2, 5.6 Hz, 1H), 2.61-2.46 (m, 1H), 2.54 (s, 3H), 2.04 (dd, J=13.1, 7.2 Hz, 1H), 1.25-1.20 (m, 3H).

¹³C NMR (126 MHz, DMSO) δ 174.50, 173.21, 170.33, 167.45, 167.19, 165.91, 165.28, 158.78, 158.49, 158.11, 155.44, 155.26, 151.26, 137.45, 137.36, 135.92, 133.50, 131.53, 129.72, 128.65, 126.50, 126.32, 120.76, 118.39, 117.23, 116.50, 115.38, 69.53, 69.16, 67.98, 59.75, 56.32, 49.28, 42.31, 38.92, 38.81, 31.41, 22.47, 15.98, 11.89.

### Example 6: Synthesis of (2R)-2-((4S)-6-(4-chlorophenyl)-8-methoxy-1-methyl-4H-benzo[f][1,2,4]triazolo [4,3-α][1,4]diazepin-4-yl)-N-(2-(2-(2-(2-((2-(2,6-dioxopiperidin-3-yl)-1,3-dioxoisoindolin-4-yl)oxy)acetamido)ethoxy)ethoxy)ethyl) propanamide (5) TFA salt

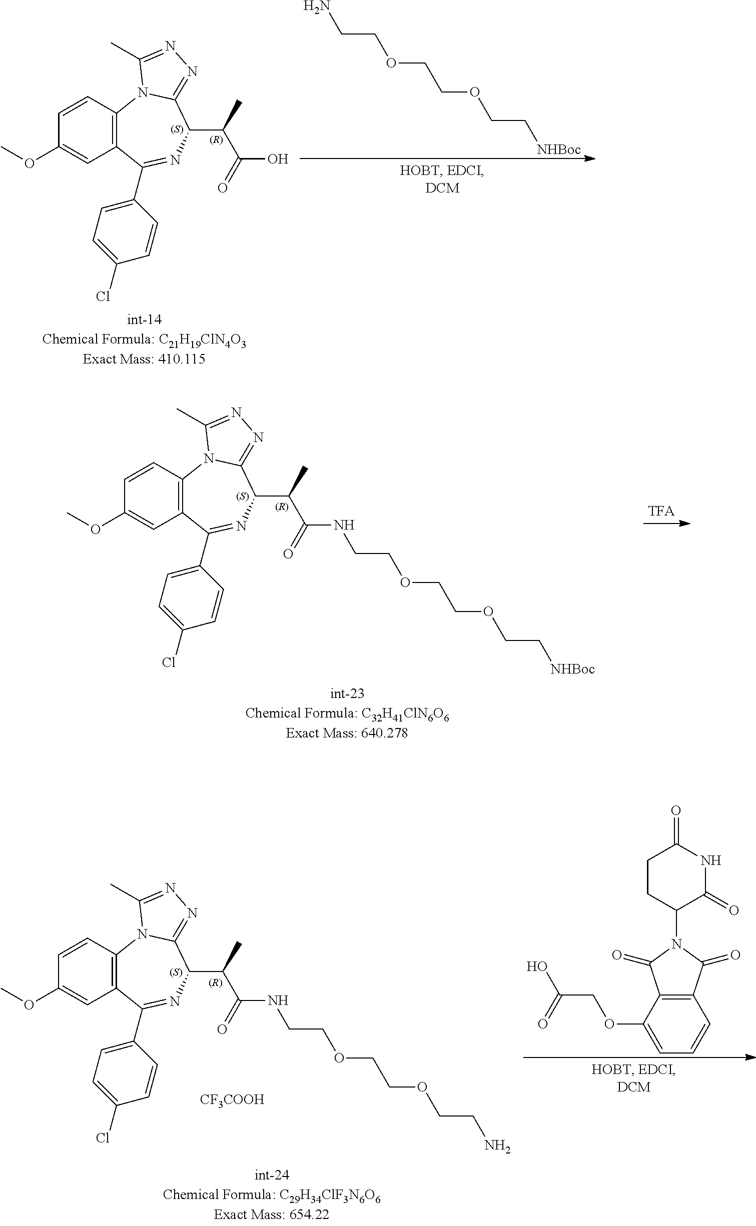

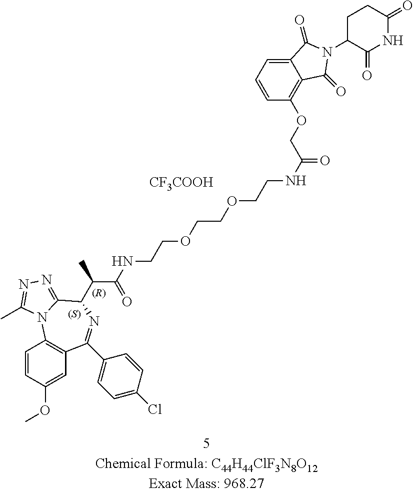

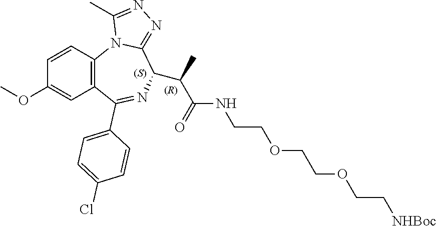

### tert-Butyl (2-(2-(2-((2R)-2-((4S)-6-(4-chlorophenyl)-8-methoxy-1-methyl-4H-benzo[f][1,2,4]triazolo[4,3-α][1,4]diazepin-4-yl)propanamido)ethoxy)ethoxy)ethyl)carbamate (int-23)

To a solution of int-14 (0.1 g, 0.244 mmol) and N-Boc-3,6-dioxaoctane-1,8-diamine (119 mg, 0.488 mmol) in dichloromethane (5 mL) were added HOBt (39 mg, 0.293 mmol) and EDC·HCL (56.2 mg, 0.293 mmol) at 0° C. The reaction mixture was stirred at room temperature under nitrogen for 2 hours, diluted with dichloromethane (30 mL), washed with brine (10 mL×2). The combined organic layers were dried over anhydrous Na₂SO₄, filtered and concentrated in vacuo. The resulting crude product was purified by flash column chromatography on silica gel (methanol in dichloromethane, 15% v/v) to afford int-23 as a white solid (0.140 g, 89.6% yield).

LC-MS (ESI) m/z: 641[M+H]⁺.

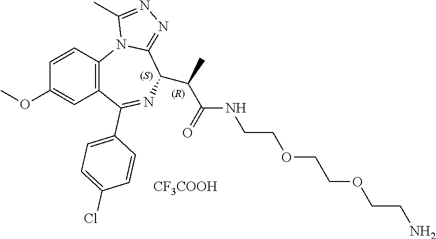

### (2R)—N-(2-(2-(2-aminoethoxy)ethoxy)ethyl)-2-((4S)-6-(4-chlorophenyl)-8-methoxy-1-methyl-4H-benzo[f][1,2,4]triazolo[4,3-α][1,4]diazepin-4-yl)propanamide TFA salt (int-24)

To a solution of int-23 (0.17 g, 0.265 mmol) in dichloromethane (3 mL) was added TFA (1 mL). The reaction mixture was stirred at room temperature for 30 minutes and concentrated in vacuo. The resulting residue was diluted with dichloromethane (30 mL), washed with saturated NaHCO₃ solution (10 mL×3) and brine (10 mL). The aqueous layers were combined and re-extracted with 10% methanol in dichloromethane (30 mL×2). The combined organic layers were dried over anhydrous Na₂SO₄, filtered and concentrated in vacuo to afford crude int-24 as an oil (0.14 g crude, 80.6% crude yield), which was used directly for the next step without further purification.

LC-MS (ESI) m/z: 541[M+H]⁺.

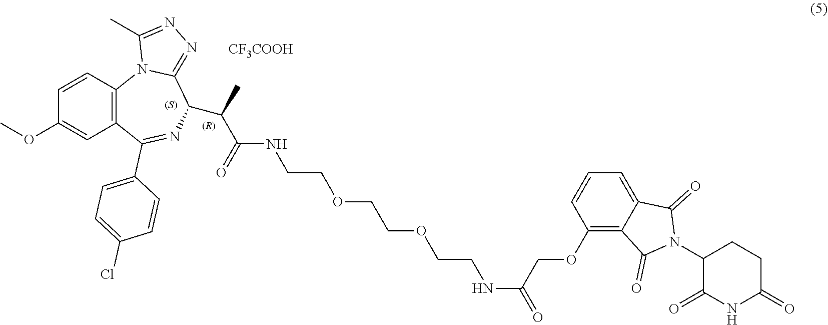

To a solution of int-24 (0.12 g crude, 0.183 mmol) and compound D (88 mg, 0.266 mmol) in anhydrous DMF (6 mL) were added HOBt (36 mg, 0.266 mmol) and EDC·HCl (51 mg, 0.266 mmol) at 0° C. The reaction mixture was stirred at room temperature under nitrogen for 2 hours, diluted with water (30 mL) and extracted with ethyl acetate (30 mL×3). The combined organic layers were washed with brine (30 mL), dried over anhydrous Na₂SO₄, filtered and concentrated in vacuo. The resulting residue was purified by preparative HPLC (MeCN—H₂O-TFA, 35%-70% gradient) to afford compound 5 as a white solid (120 mg, 67.6% yield).

LC-MS (ESI) m/z: 855[M+H]⁺.

¹H NMR (500 MHz, DMSO-d₆) δ 11.11 (s, 1H), 8.36 (t, J=5.7 Hz, 1H), 8.02 (t, J=5.7 Hz, 1H), 7.81 (d, J=9.0 Hz, 1H), 7.80 (dd, J=8.4, 7.3 Hz, 1H), 7.50-7.44 (m, 5H), 7.40 (dd, J=9.0, 2.9 Hz, 1H), 7.39 (d, J=8.8 Hz, 1H), 6.86 (d, J=2.9 Hz, 1H), 5.11 (ddd, J=12.7, 5.5, 1.7 Hz, 1H), 4.78 (s, 2H), 4.11 (d, J=10.7 Hz, 1H), 3.79 (s, 3H), 3.69 (dq, J=10.7, 6.7 Hz, 1H), 3.57 (td, J=4.6, 2.2 Hz, 4H), 3.50 (dt, J=14.7, 5.9 Hz, 4H), 3.41-3.21 (m, 4H), 2.95-2.82 (m, 1H), 2.65-2.48 (m, 2H), 2.54 (s, 3H), 2.09-1.98 (m, 1H), 1.21 (d, J=6.8 Hz, 3H).

¹³C NMR (126 MHz, DMSO) δ 174.01, 172.76, 169.86, 166.91, 166.72, 165.43, 164.75, 158.30, 158.01, 157.61, 154.98, 154.78, 150.77, 137.00, 136.91, 135.43, 133.03, 131.08, 129.22, 128.18, 126.06, 125.85, 120.33, 117.88, 116.76, 116.03, 114.92, 69.62, 69.62, 69.34, 68.85, 67.50, 59.27, 55.84, 48.80, 41.82, 38.53, 38.39, 30.94, 22.00, 15.50, 11.41.

### Example 7: Protein Purification

Wild-type and mutant BRD4_{BD1}, BRD4_{BD2} and BRD2_{BD1}L383V were obtained from Twist Bioscience in pNIC-Bio2 vector with N-term TEV cleavable His₆ tag and C-term Avi tag and all expressed in BL21-DE3 Rosetta cells using standard protocols. For purification His₆ tagged proteins cells were resuspended in buffer containing 50 mM tris(hydroxymethyl)aminomethane hydrochloride (Tris-HCl) pH 8.0, 200 mM NaCl, 1 mM tris(2-carboxyethyl)phosphine (TCEP), 1 mM phenylmethylsulfonyl fluoride (PMSF), 1× protease inhibitor cocktail (Sigma®) and lysed by sonication. Following ultracentrifugation, the soluble fraction was passed over Ni Sepharose® 6 Fast Flow affinity resin (GE Healthcare) and eluted with wash buffer (50 mM Tris-HCl pH 8.0, 200 mM NaCl, 1 mM TCEP) supplemented with 100 mM imidazole (Fischer Chemical™). The affinity-purified protein was subjected to size exclusion chromatography in 50 mM HEPES pH 7.4, 200 mM NaCl and 1 mM TCEP. Proteins were biotinylated overnight as previously described³³. The protein-containing fractions were concentrated using ultrafiltration (Millipore™) and flash frozen in liquid nitrogen at ˜25-100 μM concentration and stored at −80° C.

### Example 8: Cellular Degradation of BRD4_{BD1} and Mutant BRD4_{BD1} L94V with Inventive Bifunctional Compounds 1 to 5 and Inhibitors 1 and 2 in 293 Cells Expressing GFP-BRD/mCherry

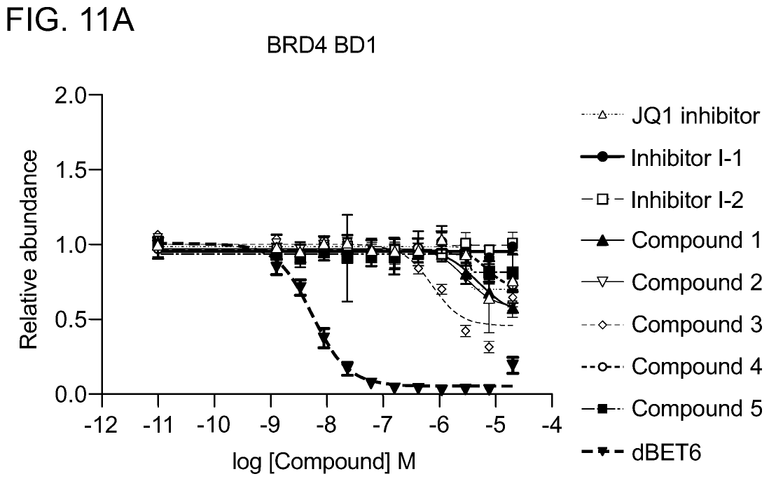

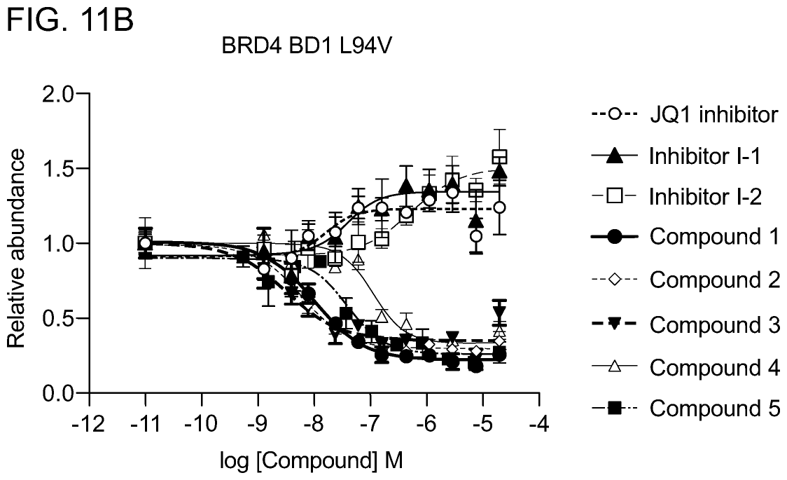

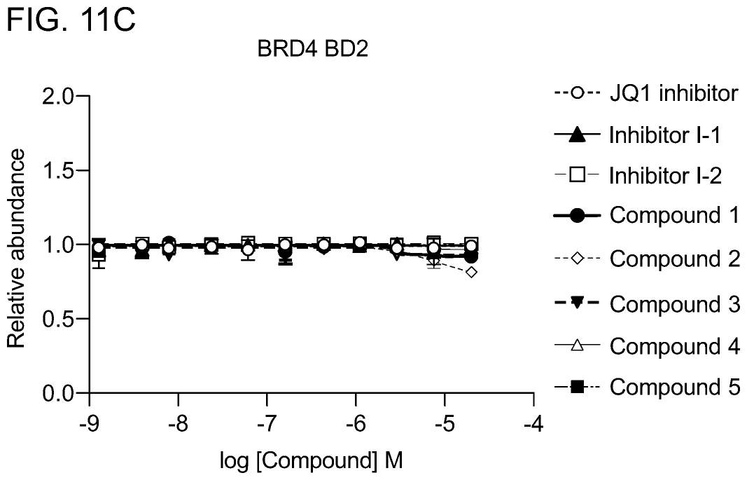

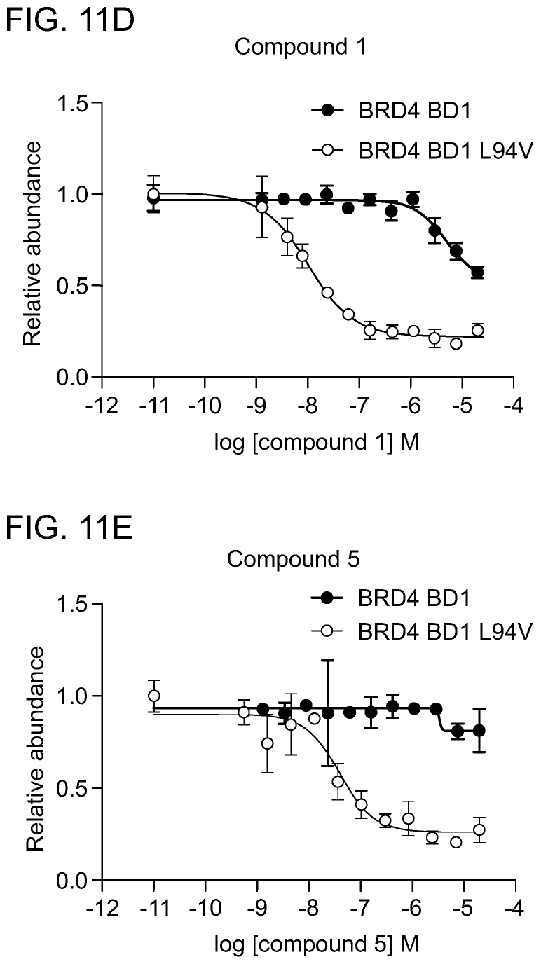

Cells stably expressing BRD4_{BD1}-EGFP or BRD4_{BD1 L94B (bumped)}-EGFP and mCherry were treated with increasing concentrations of JQ1, inventive bifunctional compounds 1 to 5, and inhibitors 1-1 and 1-2 and the EGFP and mCherry signals followed using laser scanning fluorimetry (FIG. 11A-FIG. 11C), Table 2). JQ1 as well as inhibitors 1-1 and 1-2 did not result in any measurable degradation of either BRD4_{BD1} or BRD4_{BD1}L94V while all inventive bifunctional compounds were able to degrade BRD4_{BD1}L94V with varying levels of selectivity against BRD4_{BD1} wild type (Table 2).

The results illustrated in FIG. 2A-FIG. 2H show a comparison between degradation of BRD4_{BD1} wild type and the dTAG BRD4_{BD1} L94V. JQ1 as well as inhibitor 1 and 2 did not show any measurable degradation of either BRD4_{BD1} or BRD4_{BD1} L94V while all degraders or inventive bifunctional compounds 1 to 5 were able to degrade BRD4_{BD1} L94V to varying levels of selectivity against BRD4_{BD1} wild type. Notably, compound 5 and compound 1 showed a selectivity window of 3 orders of magnitude, with compound 5 selectively degrading BRD4_{BD1 L94V} over BRD4_{BD1} wild-type. For example, no degradation of BRD4_{BD1} was observed at 1 μM concentration while the dTAG BRD4_{BD1} L94V was ˜75% degraded. These results confirmed the selective degradation of mutant BRD4_{BD1} over BRD4_{BD1} wild-type with the inventive bifunctional compounds.

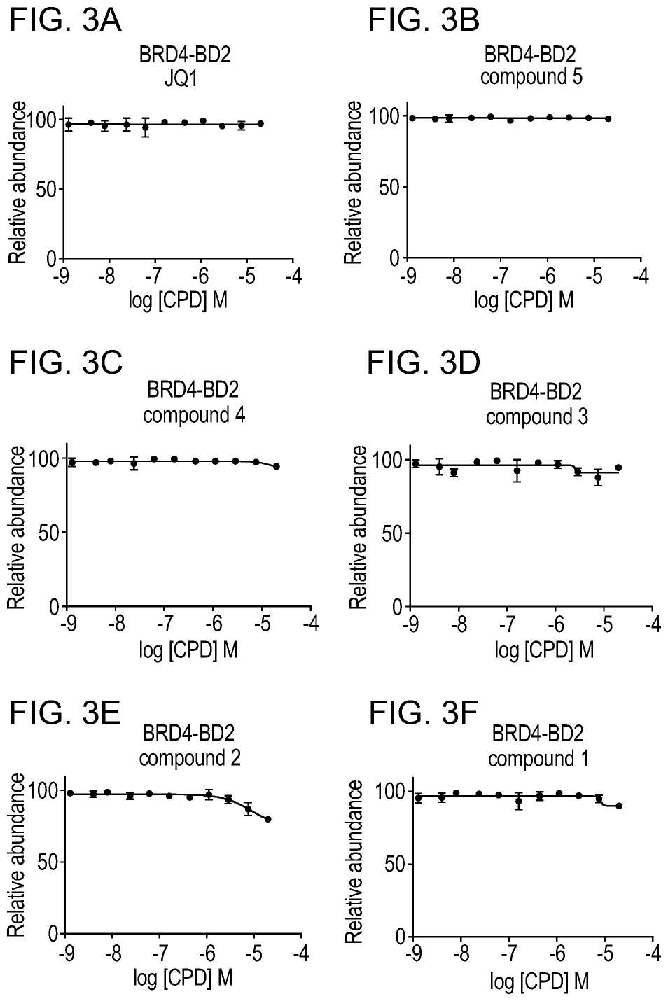

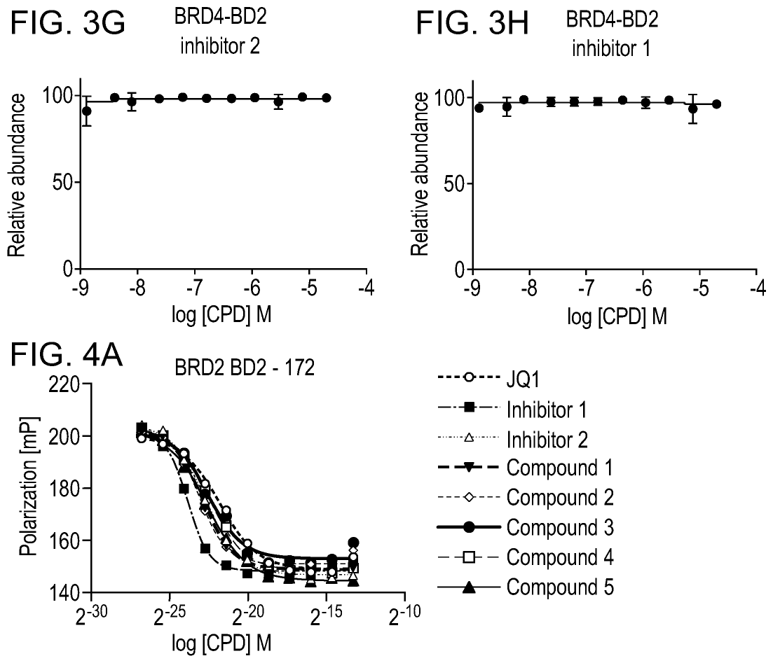

The results illustrated in FIG. 3A-FIG. 3I show that all tested inhibitors (JQ1, 1-1 and 1-2) as well as bifunctional compounds 1 to 5 are unable to degrade BRD4_{BD2} wild type.

As a comparison to bifunctional compounds 1 and 5, known degrader of BRD4_{BD1}, dBET6, was tested, and potent degradation with DC_{50, 6h} of 5.1±0.6 nM (mean±S.E.) was observed. These results confirmed the selective degradation of mutant BRD4_{BD1}L94V over BRD4_{BD1} wild type with these bifunctional compounds. Interestingly, only bifunctional compound 2 showed approximately 10% degradation of BRD4_{BD2} at highest tested concentration of 10 μM, and other degraders were unable to degrade wild type BRD4_{BD2} demonstrating exceptional selectivity of BRD4_{BD1}L94V over BRD4_{BD2}.

TABLE 2
DC₅₀ of inventive bifunctional compounds
1-5 after 6-hour incubation.

| | 1 | 2 | 3 | 4 | 5 |
|---|---|---|---|---|---|
| BRD4 BD1 | >10,000 | >10,000 | 1840 ± | >10,000 | >10,000 |
| | | | 160 | | |
| BRD4 BD1 | 10.2 ± 1.8 | 6.1 ± 0.9 | 4 ± 1 | 112 ± 20 | 40 ± 10 |
| L94V | | | | | |
| BRD4 BD2 | >20,000 | >20,000 | >20,000 | >20,000 | >20,000 |
| Fold | >1000 | >1,600 | 460 | >89 | >250 |
| selectivity | | | | | |
| BRD4 BD1/ | | | | | |
| BD1 L94V | | | | | |

Table 2 shows calculated DC_{50,6h} values in nM for BRD4_{BD1}, BRD4_{BD1}L94V and BRD4_{BD2} reporter cell lines as well as calculated fold-degradation selectivity for BRD4_{BD1} and corresponding BRD4_{BD1}L94V mutant. Values are shown as DC_{50, 6h}±S.E. (standard error) of N=3 replicates for BRD4_{BD1} and BRD4_{BD1}L94V, and N=2 for BRD4_{BD2}. The results in Table 2 show that selective BRD4_{BD1L94V} degradation was achieved to varying extend by compounds 1 to 5. Notably, compounds 1 and 5 showed 1000- and 250-fold selectivity, respectively.

### Example 9: Fluorescence Polarization Assays

Compounds in tetramethylrhodamine (TAMRA)-conjugated JQ1 (TAMRA-JQ1) displacement assay were dispensed in a 384-well microplate (Corning™, 4514) using D300e Digital Dispenser (HP) normalized to 1% DMSO into 10 nM TAMRA-JQ1, 50 mM Tris pH 7.5, 200 mM NaCl, 0.1% Pluronic® F-68 solution (Sigma) with 400 nM BRD4_{BD1 L94V} or 250 nM BRD4_{BD2 L387V} or 150 nM BRD2_{BD2 L383V} or 50 nM BRD4_{BD2}. The change in fluorescence polarization was monitored using a PHERAstar® FS microplate reader (BMG Labtech) until a stable signal was observed. Dose response data (N=1) was plotted and IC₅₀ values were estimated using variable slope equation in GraphPad Prism 7.

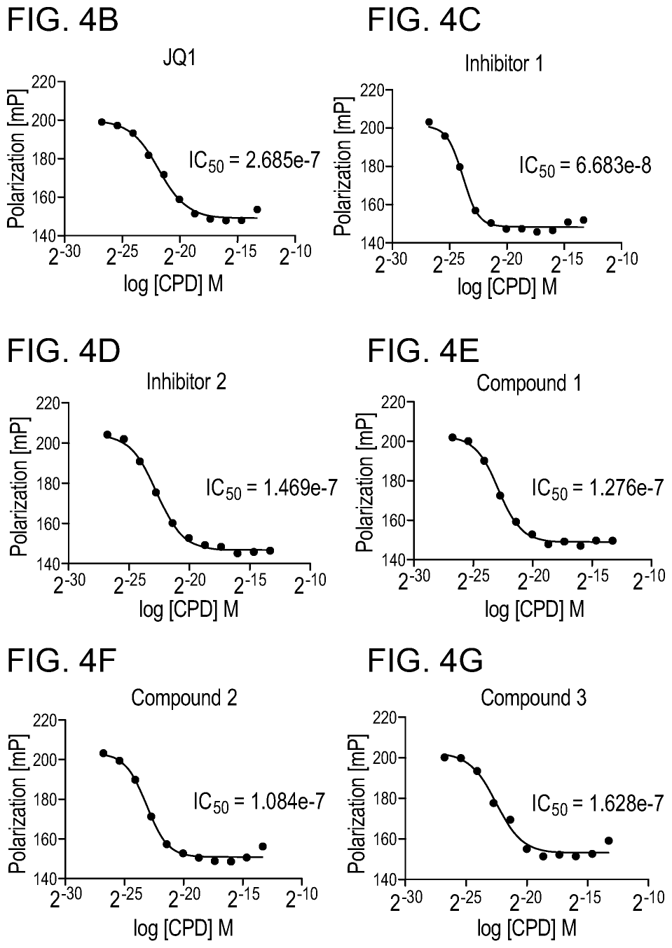

The results illustrated in FIG. 4A-FIG. 41 show potent binding of JQ1 and bumped analogs (inhibitors 1-1 and 1-2), as well as bifunctional compounds 1 to 5. Notably, 1-1 was 4-fold more selective in binding BRD2_{BD2 L383V} than JQ1. As shown in Table 2, all degraders derived from inhibitors 1-1 and 1-2 showed more potent binding to BRD2_{BD2 L383V} than the JQ1.

TABLE 2
IC₅₀ of inventive bifunctional compounds 1-5 and inhibitors 1 and 2.

| Compound | JQ1 | I-1 | I-2 | 1 | 2 | 3 | 4 | 5 |
|---|---|---|---|---|---|---|---|---|
| IC₅₀ (M) | 2.685E−07 | 6.683E−08 | 1.469E−07 | 1.276E−07 | 1.084E−07 | 1.628E−07 | 1.700E−07 | 1.533E−07 |

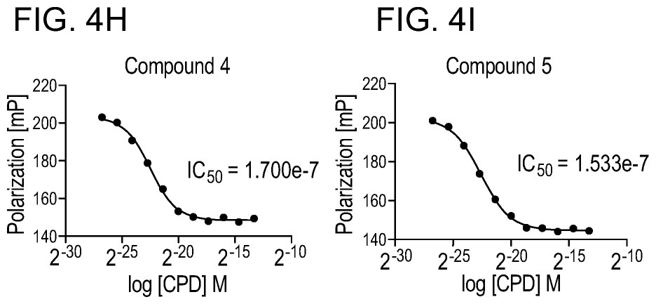

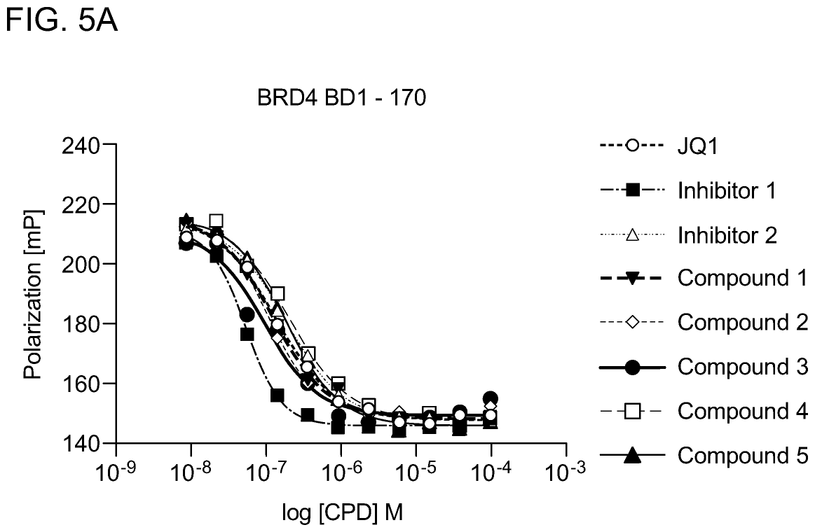

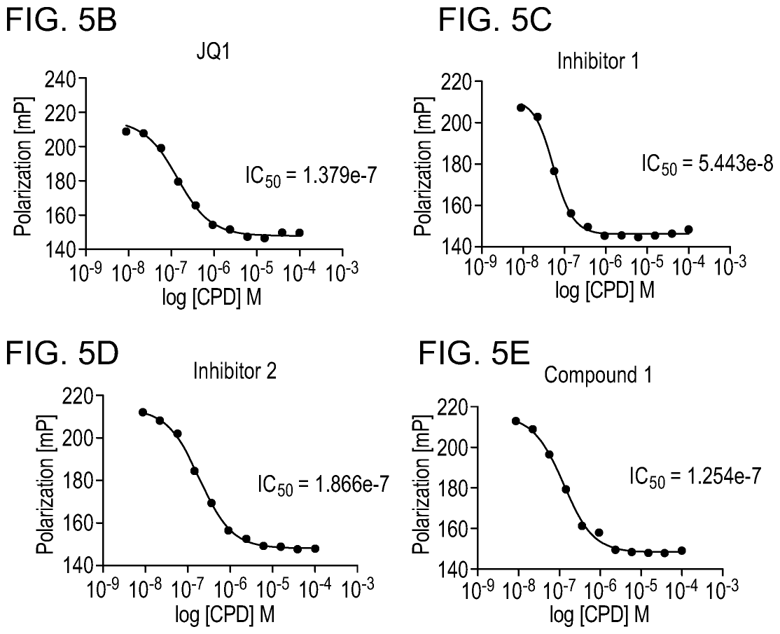

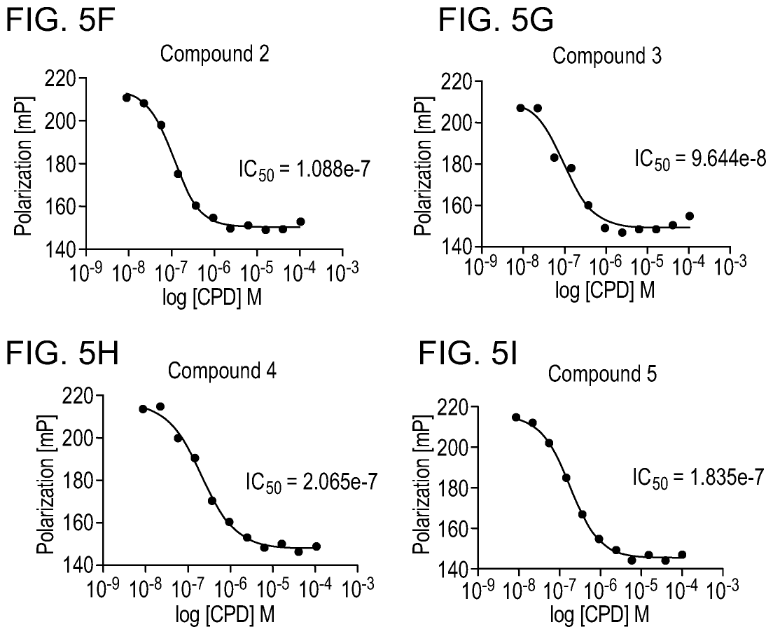

The results in Table 2 show that all tested compounds potently bound to BRD2_{BD2 L383V}. The results illustrated in FIG. 5A-FIG. 51 show potent binding of JQ1 and bumped analogs (inhibitors I-1 and I-2), as well as bifunctional compounds 1 to 5 to BRD4_{BD1 L94V}. Notably, as shown in Table 3, I-1 was more than 2-fold more potent than JQ1 in binding to BRD4_{BD1 L94V}.

TABLE 3
IC₅₀ of inventive bifunctional compounds 1-5 and inhibitors 1 and 2.

| Compound | JQ1 | I-1 | I-2 | 1 | 2 | 3 | 4 | 5 |
|---|---|---|---|---|---|---|---|---|
| IC₅₀ (M) | 1.379E−07 | 5.443E−08 | 1.866E−07 | 1.254E−07 | 1.088E−07 | 9.644E−08 | 2.065E−07 | 1.835E−07 |

The results in Table 3 also show that all tested compounds potently bound to BRD4_{BD1} L94V.

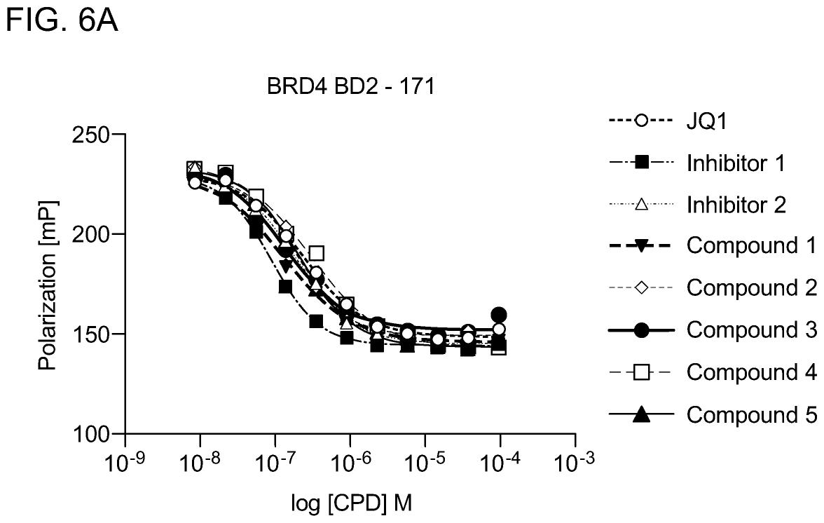

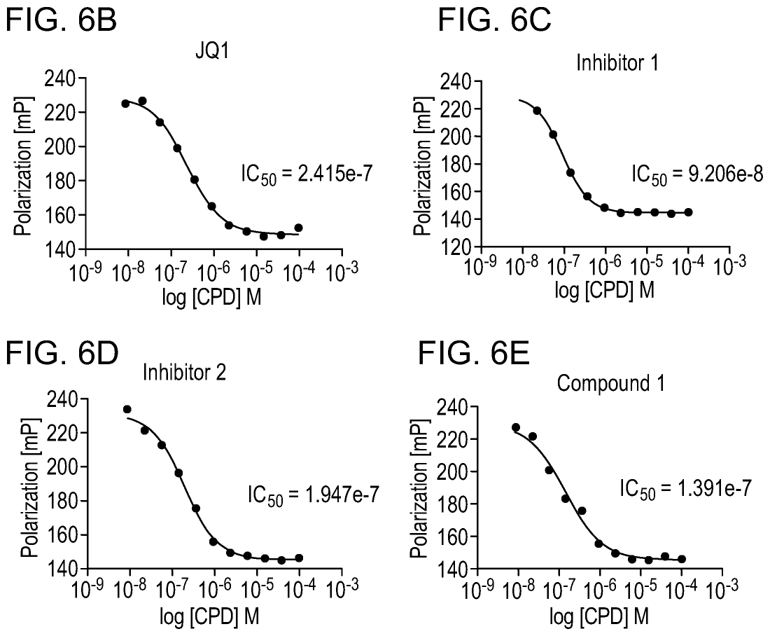

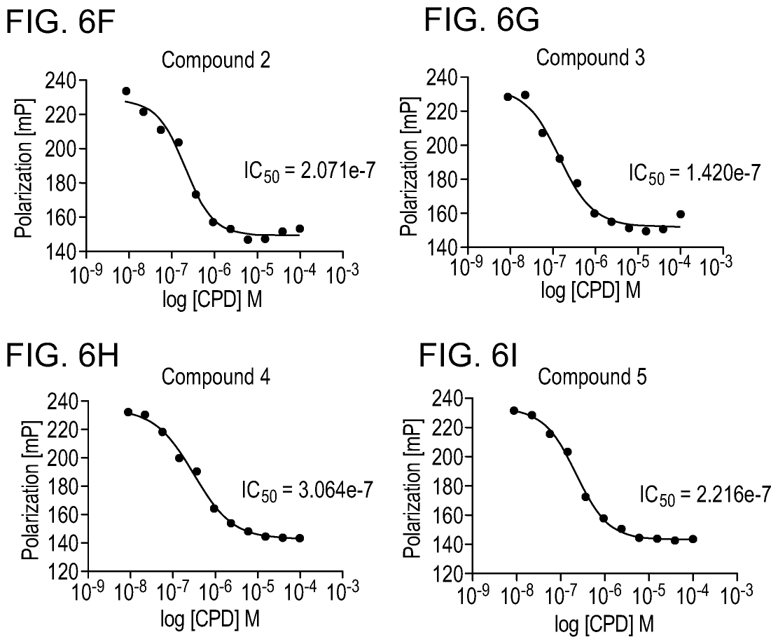

The results illustrated in FIG. 6A-FIG. 61 show that potent binding was achieved to BRD4_{BD2} L387V. Notably, I-1 showed enhanced potency (about 2-fold) as compared to JQ1.

TABLE 4
IC₅₀ of inventive bifunctional compounds 1-5 and inhibitors 1 and 2.

| Compound | JQ1 | I-1 | I-2 | 1 | 2 | 3 | 4 | 5 |
|---|---|---|---|---|---|---|---|---|
| IC₅₀ (M) | 2.415E−07 | 9.206E−08 | 1.947E−07 | 1.391E−07 | 2.071E−07 | 1.420E−07 | 3.064E−07 | 2.216E−07 |

The results in Table 4 also show that all tested compounds potently bound to BRD4_{BD1 L94V}.

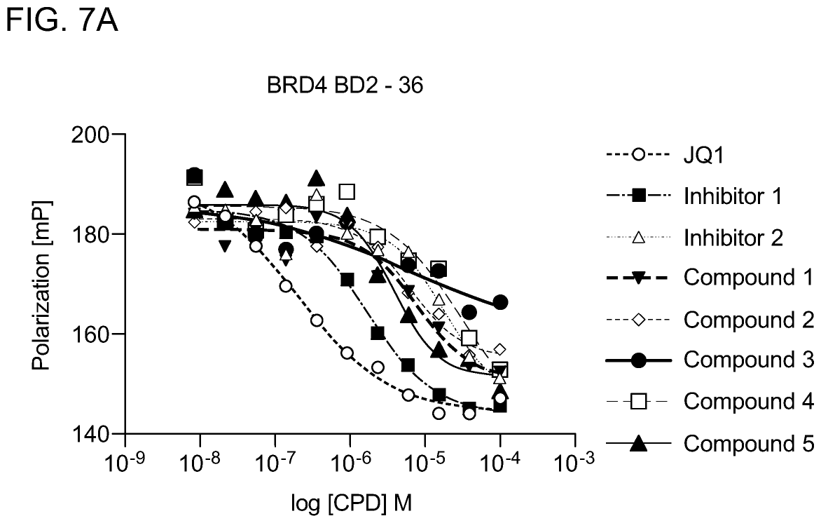

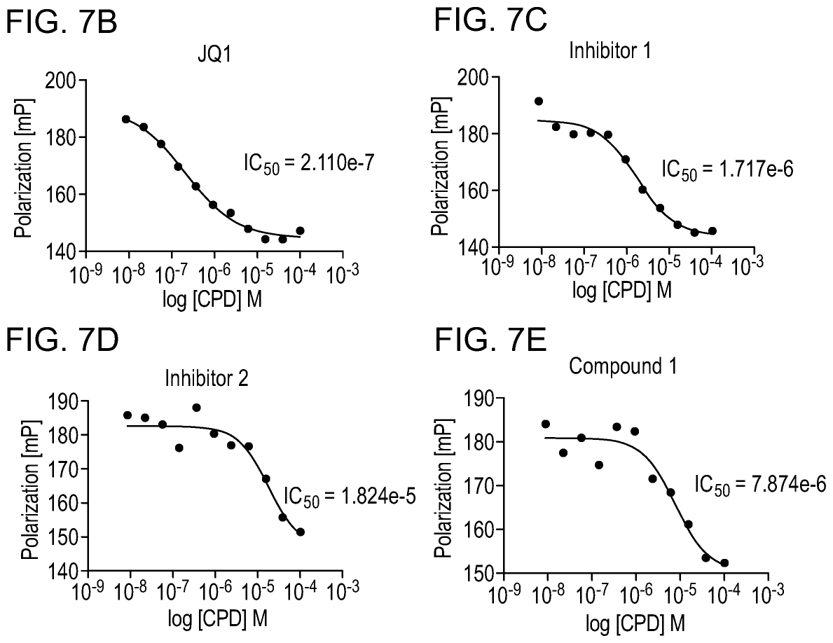

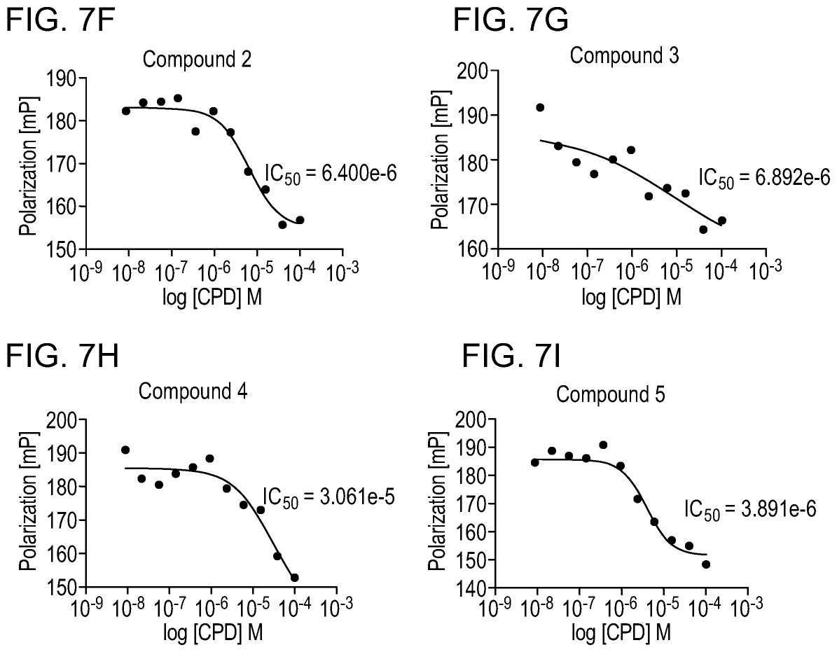

The results illustrated in FIG. 7A-FIG. 71 show significantly reduced affinity of all bumped inhibitors (inhibitors 1 and 2) and degraders (compounds 1-5) to wild type BRD4_{BD2}. As expected, JQ1 was the most potent binder. Inability to engage with BRD4 at low concentrations impaired formation of the ternary complex and therefore significantly reduced potency in degradation.

TABLE 5
IC₅₀ of inventive bifunctional compounds 1-5 and inhibitors 1 and 2.

| Compound | JQ1 | I-1 | I-2 | 1 | 2 | 3 | 4 | 5 |
|---|---|---|---|---|---|---|---|---|
| IC₅₀ (M) | 2.110E−07 | 1.717E−06 | 1.824E−05 | 7.874E−06 | 6.400E−06 | 6.892E−06 | 3.061E−05 | 3.891E−06 |

The results illustrated in Table 5 show that both inhibitors I-1 and I-2 have significantly reduced affinity to wild type BRD4_{BD2} with IC₅₀ values ranging from 1.7 to 18 μM. The same was observed for the degrader molecules exemplified in bifunctional compounds 1 to 5 with IC₅₀ values ranging from 6.4 μM for compound 2 to 30 μM for compound 4. JQ1 was able to potently bind BRD4_{BD2} with an IC₅₀ value of about 211 nM.

The results of whole proteome mass spectrometry profiling show that inventive compounds 1 and 5 did not degrade any native BET bromodomains, or any other unrelated off targets in MOLT4 cells after 6-hour treatment with 1 μM compound 1 or 5. See, Example 12.

### Example 10: Selective Degradation of BRD4_{BD1 L94V} by Inventive Bifunctional Compounds 1 and 5

### Sample Preparation TMT LC-MS3 Mass Spectrometry

MOLT4 cells were treated with DMSO (biological triplicates) or 1 μM of bifunctional compound 5 or 1 μM of 1 (in biological singlicate) in for 5 hours and cells harvested by centrifugation. Lysis buffer (8 M Urea, 50 mM NaCl, 50 mM 4-(2hydroxyethyl)-1-piperazineethanesulfonic acid (EPPS) pH 8.5, Protease and Phosphatase inhibitors from Roche®) were added to the cell pellets and homogenized by 20 passes through a 21 gauge (1.25 in. long) needle to achieve a cell lysate with a protein concentration between 1-4 mg mL⁻¹. A micro-BCA assay (Pierce™) was used to determine the final protein concentration in the cell lysate. 200 μg of protein for each sample were reduced and alkylated as previously described in Donovan et al., Elife 7: e38430 (2018).

Proteins were precipitated using methanol/chloroform. In brief, four volumes of methanol were added to the cell lysate, followed by one volume of chloroform, and finally three volumes of water. The mixture was vortexed and centrifuged to separate the chloroform phase from the aqueous phase. The precipitated protein was washed with three volumes of methanol, centrifuged and the resulting washed precipitated protein was allowed to air dry. Precipitated protein was resuspended in 4 M Urea, 50 mM 2-[4-(2-hydroxyethyl)piperazin-1-yl]ethanesulfonic acid (HEPES) pH 7.4, followed by dilution to 1 M urea with the addition of 200 mM EPPS, pH 8. Proteins were first digested with LysC (1:50; enzyme:protein) for 12 hours at room temperature. The LysC digestion was diluted to 0.5 M Urea with 200 mM EPPS pH 8 followed by digestion with trypsin (1:50; enzyme:protein) for 6 hours at 37° C. Tandem mass tag (TMT) reagents (Thermo Fisher Scientific) were dissolved in anhydrous acetonitrile (ACN) according to manufacturer's instructions. Anhydrous ACN was added to each peptide sample to a final concentration of 30% v/v, and labeling was induced with the addition of TMT reagent to each sample at a ratio of 1:4 peptide:TMT label. The 10-plex labeling reactions were performed for 1.5 hours at room temperature and the reaction quenched by the addition of hydroxylamine to a final concentration of 0.3% for 15 minutes at room temperature.

The sample channels were combined at a 1:1:1:1:1:1:1:1:1:1:1 ratio, desalted using C₁₈ solid phase extraction cartridges (Waters®) and analyzed by LC-MS for channel ratio comparison. Samples were then combined using the adjusted volumes determined in the channel ratio analysis and dried down in a speed vacuum. The combined sample was then resuspended in 1% formic acid, and acidified (pH 2-3) before being subjected to desalting with C18 SPE (Sep-Pak®, Waters®). Samples were then offline fractionated into 96 fractions by high pH reverse-phase HPLC (Agilent LC1260) through an aeris peptide xb-c18 column (Phenomenex®) with mobile phase A containing 5% acetonitrile and 10 mM NH₄HCO₃ in LC-MS grade H₂O, and mobile phase B containing 90% acetonitrile and 10 mM NH₄HCO₃ in LC-MS grade H₂O (both pH 8.0). The 96 resulting fractions were then pooled in a non-continuous manner into 24 fractions and these fractions were used for subsequent mass spectrometry analysis.

Data were collected using an Orbitrap Fusion™ Lumos™ mass spectrometer (Thermo Fisher Scientific, San Jose, Calif., USA) coupled with a Proxeon EASY-nLC™ 1200 LC pump (Thermo Fisher Scientific). Peptides were separated on an EasySpray™ ES803 75 μm inner diameter microcapillary column (ThermoFisher Scientific). Peptides were separated using a 190 min gradient of 6-27% acetonitrile in 1.0% formic acid with a flow rate of 300 nL/min.

Each analysis used an MS3-based TMT method as described previously in McAlister et al., Anal. Chem. 86:7150-7158 (2014). The data were acquired using a mass range of m/z 340-1350, resolution 120,000, automatic gain control (AGC) target 5×105, maximum injection time 100 ms, dynamic exclusion of 120 seconds for the peptide measurements in the Orbitrap. Data dependent MS2 spectra were acquired in the ion trap with a normalized collision energy (NCE) set at 35%, AGC target set to 1.8×104 and a maximum injection time of 120 ms. MS3 scans were acquired in the Orbitrap with a HCD collision energy set to 55%, AGC target set to 2×105, maximum injection time of 150 ms, resolution at 50,000 and with a maximum synchronous precursor selection (SPS) precursors set to 10.

LC-MS data analysis.

Proteome Discoverer 2.2 (Thermo Fisher Scientific) was used for .RAW file processing and controlling peptide and protein level false discovery rates, assembling proteins from peptides, and protein quantification from peptides. MS/MS spectra were searched against a Uniprot human database (September 2016) with both the forward and reverse sequences. Database search criteria are as follows: tryptic with two missed cleavages, a precursor mass tolerance of 20 ppm, fragment ion mass tolerance of 0.6 Da, static alkylation of cysteine (57.02146 Da), static TMT labelling of lysine residues and N-termini of peptides (229.16293 Da), and variable oxidation of methionine (15.99491 Da). TMT reporter ion intensities were measured using a 0.003 Da window around the theoretical m/z for each reporter ion in the MS3 scan. Peptide spectral matches with poor quality MS3 spectra were excluded from quantitation (summed signal-to-noise across 11 channels <100 and precursor isolation specificity <0.5), and resulting data was filtered to only include proteins that had a minimum of 2 unique peptides identified. Reporter ion intensities were normalized and scaled using in-house scripts in the R framework (R Development Core Team (2008). R: A language and environment for statistical computing. R Foundation for Statistical Computing, Vienna, Austria. ISBN 3-900051-07-0). Statistical analysis was carried out using the limma package within the R framework (Ritchie et al., Nucleic Acids Res. 43:e47 (2015).

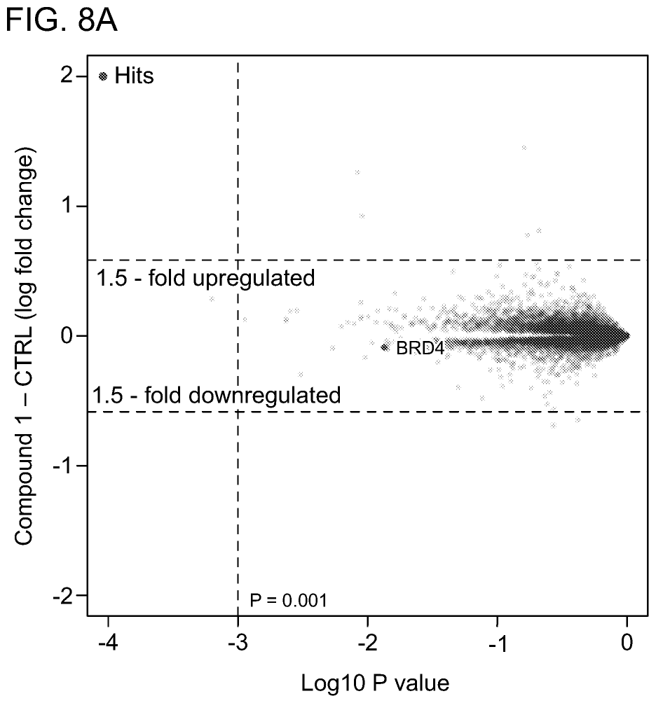

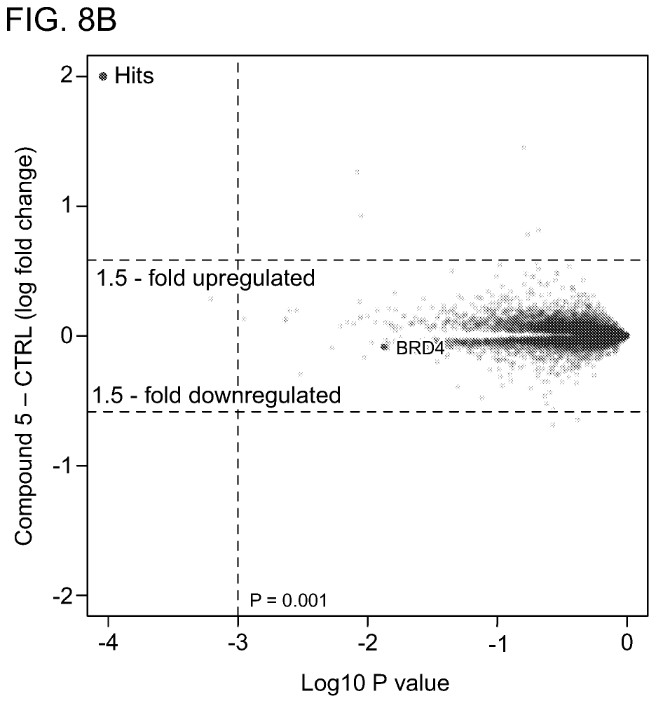

The results are illustrated in FIG. 8A-FIG. 8B. They show the elective degradation of BRD4_{BD1 L94V} by inventive bifunctional compounds 1 and 5 in MOLT4 cells. While both bifunctional compounds 1 and 5 were able to potently degrade BRD4_{BD1 L94V} in a GFP reporter cell line, no native off target degradation was detected in whole proteome mass spectrometry across ˜7,800 quantified proteins, including the BET family of bromodomains, after 6 h incubation experiment with both bifunctional compounds. This experiment confirmed the selectivity of the inventive bifunctional compounds. LIMK2 appeared to be the only protein upregulated in response to treatment with bifunctional compound 5, but its expression was unchanged in response to bifunctional compound 1. We have seen similar data in MOLT4 cells and anticipate that this may be a result of experimental variability rather than a definitive biological stabilization effect.

### Example 11: Proof-of-Principle Simultaneous Use of dTAG and BRD4_{BD1}L94V Degron Systems

Synthetic lethality, often discovered using CRISPR/Cas9 based functional genomic screening strategies, can be a very powerful strategy to target cancer (Farmer et al., Nature 434(7035):917-21 (2005)). As many of the discovered co-dependencies lack available chemical tools for target validation, a degradation method enabling two degraders to simultaneously modulate protein levels in cells was explored.

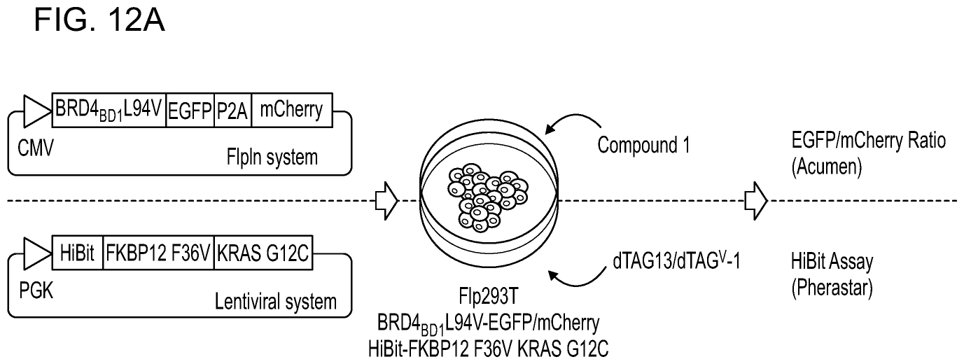

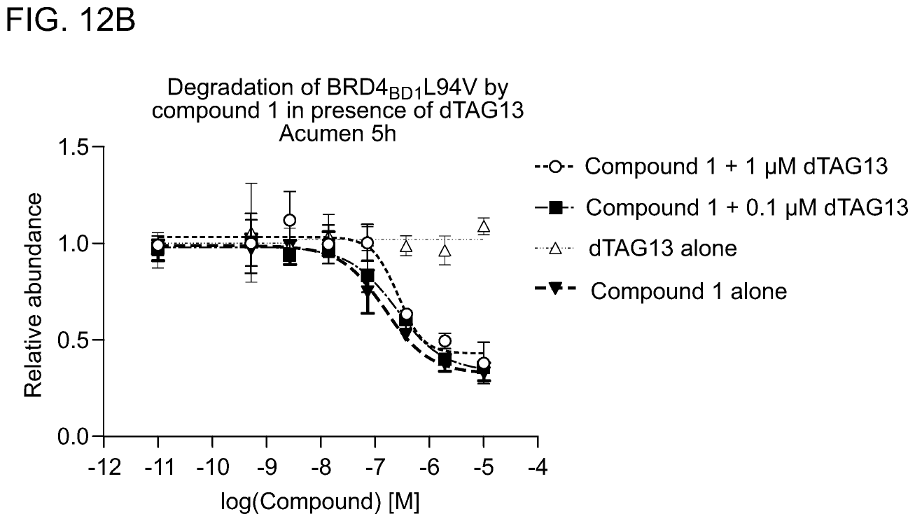

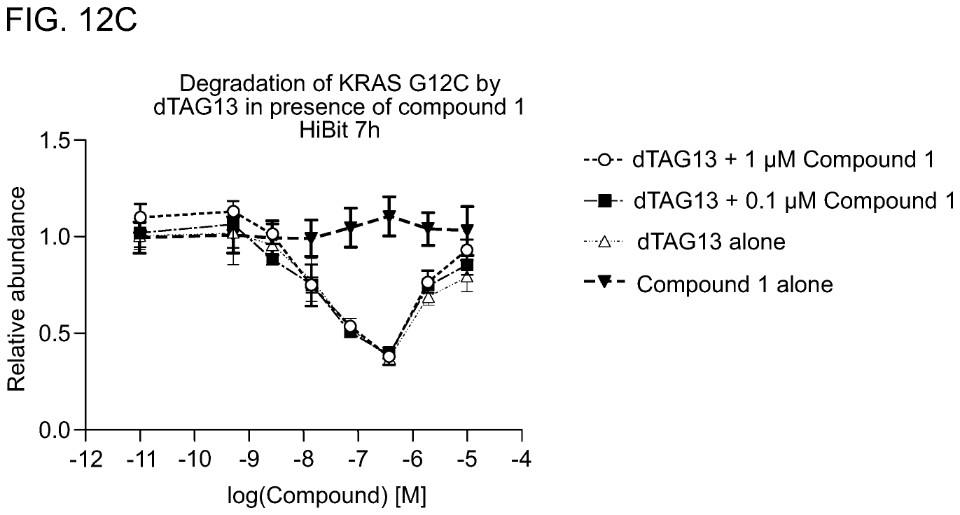

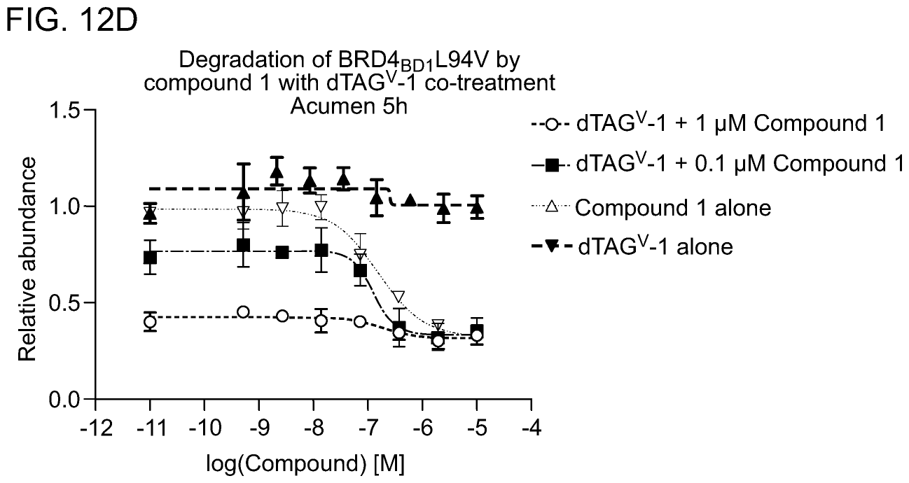

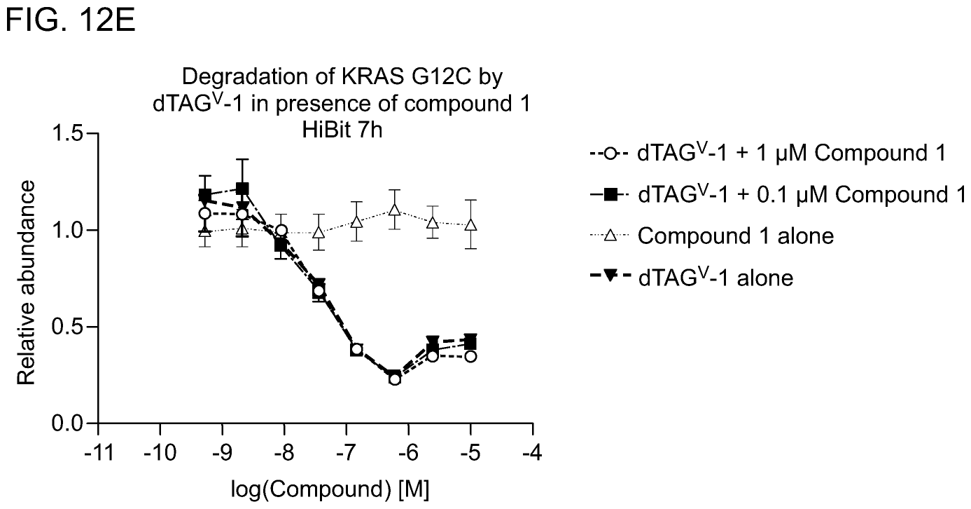

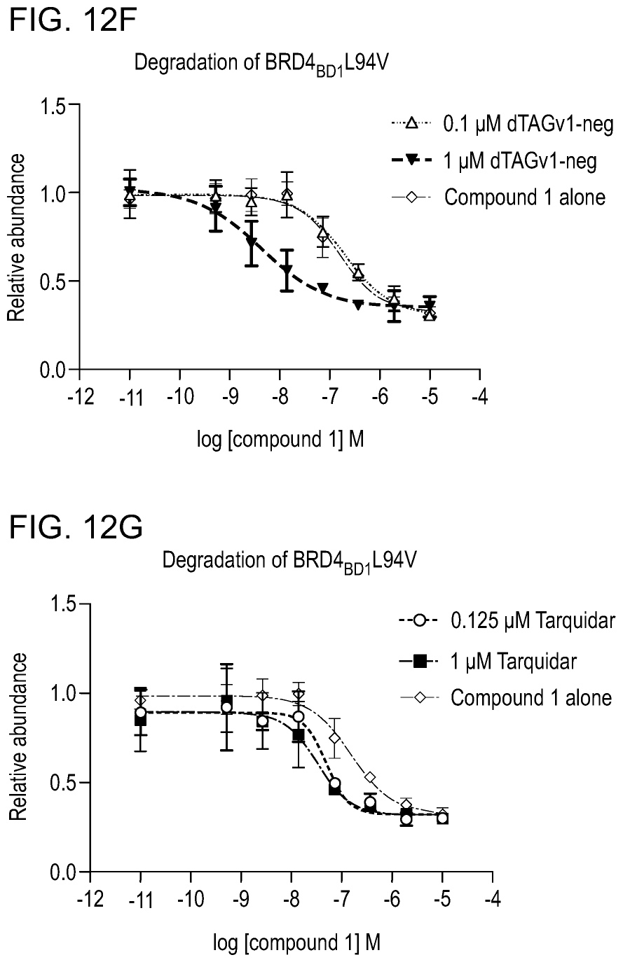

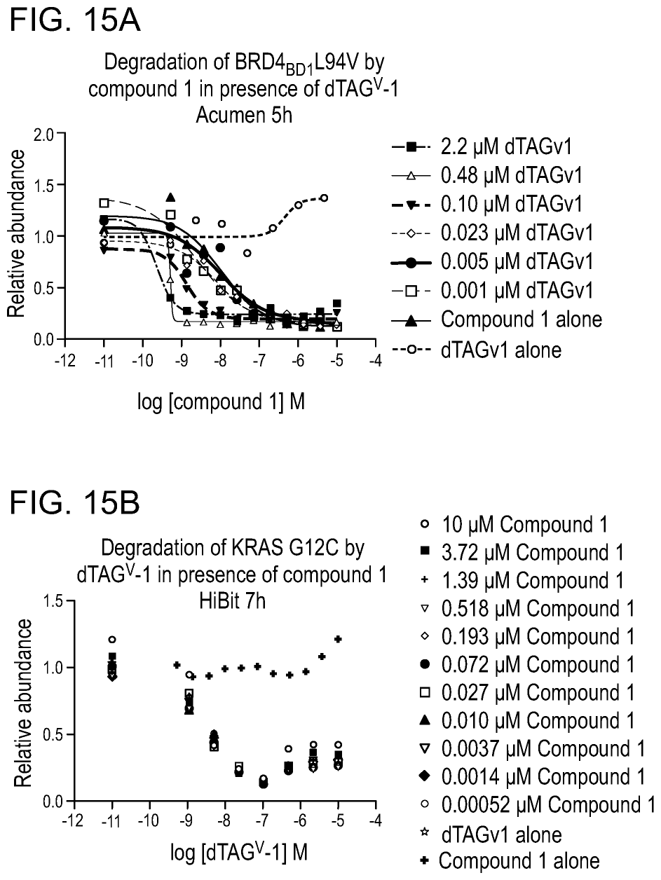

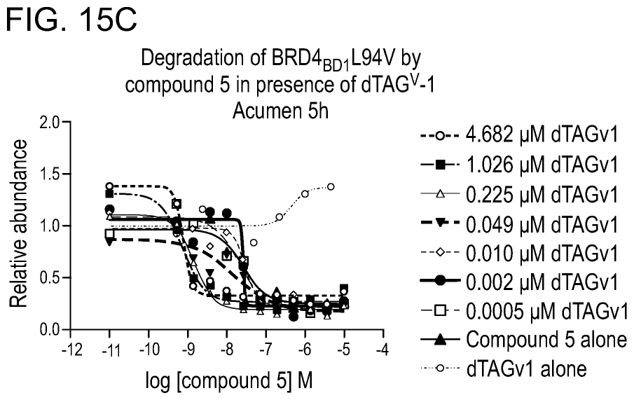

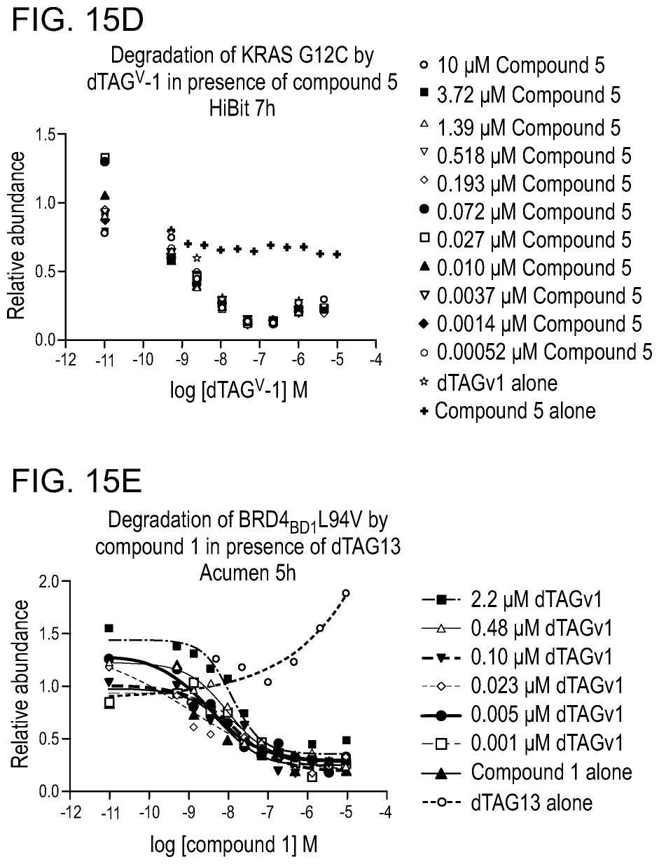

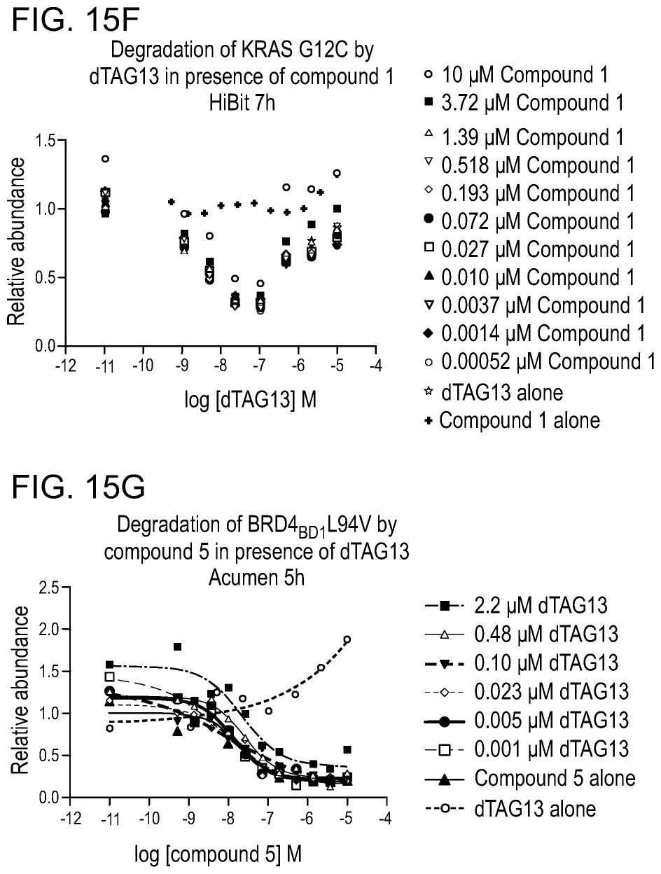

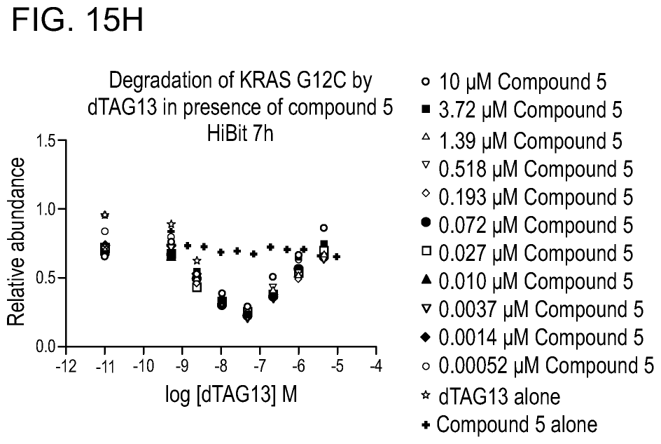

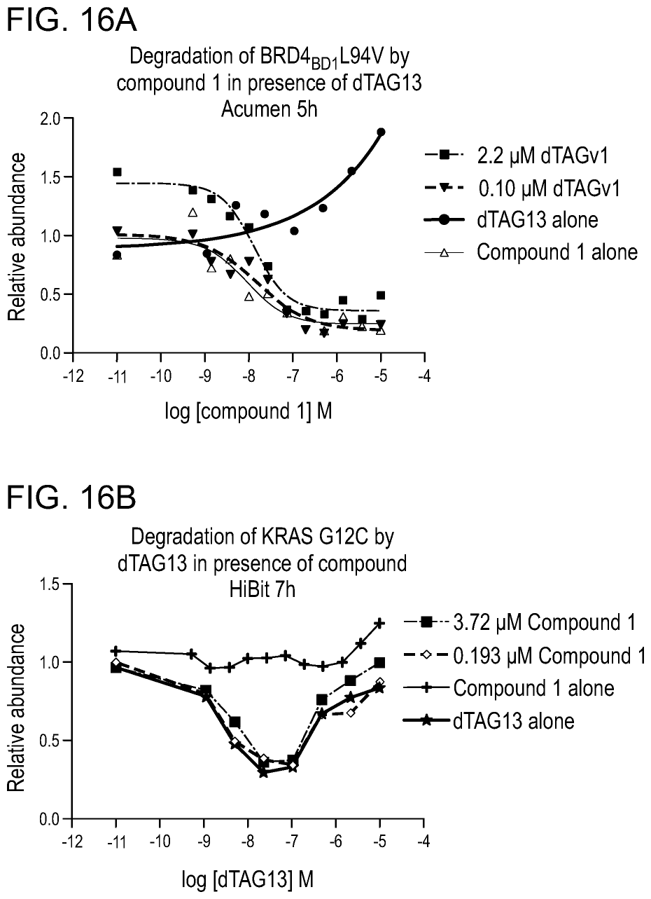

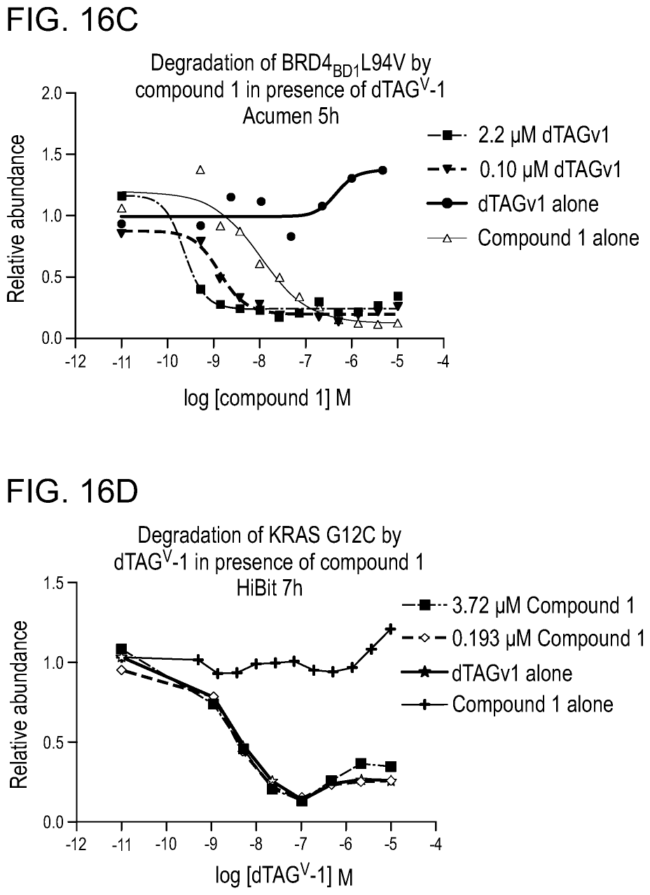

To establish proof-of-principle, a dual reporter line by combining the BRD4_{BD1}L94V degron with the dTAG system was generated (FIG. 12A). The previously characterised BRD4_{BD1} L94V-EGFP/mCherry reporter cell line and stably expressed a HiBit-FKBP12 F36V-KRAS G12C fusion protein using lentiviral-mediated stable integration were used. The abundance of each protein was measured by quantification of EGFP/mCherry ratio for BRD4_{BD1} L94V and a HiBit lytic assay for the HiBit tagged FKBP12 F36V-KRAS G12C. In order to validate the simultaneous degradation of protein levels in the dual reporter line, co-treatment of bifunctional compound 1 or 5 with CRBN based degrader dTAG13 or VHL based degrader dTAG^{v}-1 was performed, and the same assay plate was analysed using laser scanning fluorimetry (Acumen®) at 5 h, followed by NanoLuc® HiBit lytic assay at 7 h (FIG. 12B-FIG. 12E, FIG. 15A-FIG. 15H, FIG. 16A-FIG. 16D).

The structures of degraders dTAG13 and dTAG^{v}-1 are shown below:

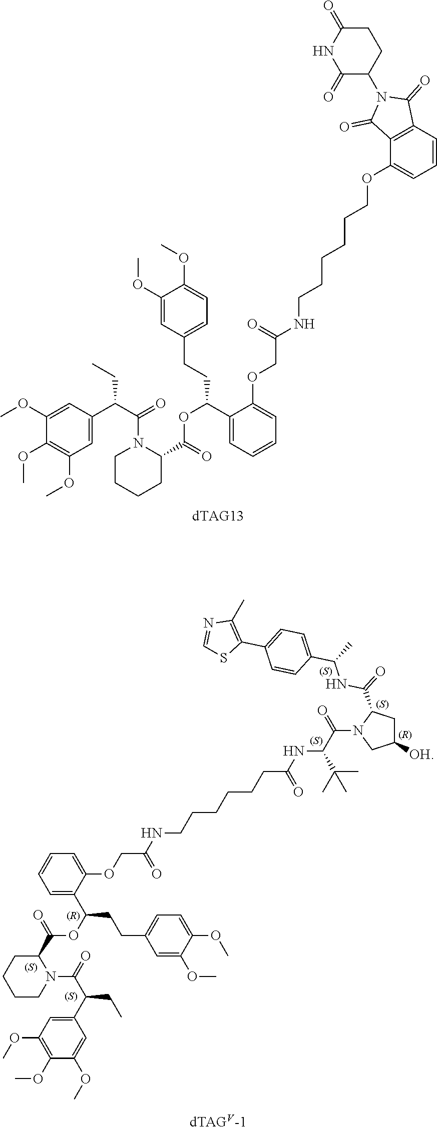

### Cellular Degradation Assays Using Acumen®

A day before compound treatment, Flp293T cells stably expressing the BRD4_{BD1}-EGFP, BRD4_{BD2}-EGFP, or BRD4_{BD1} L94V-EGFP with mCherry reporter established using the Flip-In™ system, as described in Nowak et al., Nat Chem Biol 14 (7):706-714 (2018), or Flp293T with simultaneous stable expression of BRD4_{BD1}L94V-EGFP with mCherry reporter, and HiBit-FKBP12 F36V-KRAS G12C were seeded at 30-50% confluency in 384-well plates with 50 μL FluoroBrite™ DMEM media (Thermo Fisher Scientific, A18967) containing 10% fetal bovine serum (FBS) per well. Compounds were dispensed using a D300e Digital Dispenser (HP), normalized to 0.5% DMSO, and incubated with cells for 5 hours. The assay plate was imaged immediately using an Acumen® High Content Imager (TTP Labtech) with 488 nm and 561 nm lasers in 2 μm×1 μm grid per well format. The resulting images were analyzed using CellProfiler (Carpenter et al., Genome Biol 7(10):R100 (2006)) or Cellista™ Acumen® software (TTP Labtech). For Cellista™ analysis, signal was quantified as a ratio of 488 nm (EGFP): Total (FL-2 Peak Intensity) and 561 nm (mCherry):Total (FL-3 Peak Intensity) and normalized to DMSO. For CellProfiler analysis, a series of image analysis steps (‘image analysis pipeline’) was constructed. First, the red and green channels were aligned and cropped to target the middle of each well (to avoid analysis of heavily clumped cells at the edges), and a background illumination function was calculated for both red and green channels of each well individually and subtracted to correct for illumination variations across the 384-well plate from various sources of error. An additional step was then applied to the green channel to suppress the analysis of large auto fluorescent artifacts and enhance the analysis of cell specific fluorescence by way of selecting for objects under a given size, 30 A.U., and with a given shape, speckles. mCherry-positive cells were then identified in the red channel filtering for objects between 8-60 pixels in diameter and using intensity to distinguish between clumped objects. The green channel was then segmented into EGFP positive and negative areas and objects were labeled as EGFP positive if at least 40% of it overlapped with a EGFP positive area. The fraction of EGFP-positive cells/mCherry-positive cells in each well was then calculated, and the green and red images were rescaled for visualization. The values for the concentrations that lead to a 50% degradation (DC₅₀) were calculated using the nonlinear fit variable slope model in GraphPad Prism software.

### Generation of the BRD4_{BD1}L94V; KRASG12C Stable Line

HEK293T cells were seeded at 700,000 cells per well in a 6-well plate (Falcon®, 353046) with DMEM media (Gibco™, Life Technologies™) containing 10% FBS with the final volume of 3 ml per well. The lentivirus was constructed by co-transfection of the expression plasmid HiBit-FKBP12 F36V-KRAS G12C based on Cilantro 2 backbone (Addgene, #74450), but with fluorescence markers removed) at 1 μg quantity together with 1 μg psPax2 (Addgene, #12260) and 0.1 μg pVSV-G (Addgene, #36399), and 4:1 ratio of FuGene® to DNA in 100 μL of Opti-MEM media (Gibco, Life Technologies) the following day. The lentivirus was harvested 48 h after transfection by centrifugation at 1000×g for 5 min and frozen at −80° C. For transductions, Flp293T cells stably expressing BRD4_{BD1}L94V-EGFP and mCherry reporter separated by P2A site were seeded in a 6-well plate at 600,000 per. Transduction was performed the next day at 40%: 800 μL of lentiviral soup was added to 1.2 mL of DMEM+10% FBS and 1.6 μl of 10 mg/ml polybrene. After 2-3 days cells with stable integration of the HiBit-FKBP12 F36V-KRAS G12C were selected using 1 μg/mL of puromycin. Final cell line Flp293T stably expressing BRD4_{BD1}L94V-EGFP-p2a-mCherry under CMV promoter and HiBit-FKBP12 F36V-KRAS G12C under PGK promoter was used for degradation experiments.

### Cellular Degradation Assays Using HiBit

Flp293T cells stably expressing BRD4_{BD1}L94V-EGFP-P2A-mCherry under cytomegalovirus (CMV) promoter and HiBit-FKBP12 F36V-KRAS G12C under phosphoglycerate kinase (PGK) promoter was seeded at 40% confluency in a 384-well plate (Corning®, 3573) at 50 μL per well in FluoroBrite™ DMEM media (Thermo Fisher Scientific, A18967) containing 10% FBS. Compounds were dispensed using D300e Digital Dispenser (HP) and normalized to 0.5% DMSO. For the simultaneous degradation study assay plate was read using Acumen® as described above at 5 h timepoint, and then using NanoGlo® HiBit lytic assay at 7 h timepoint. HiBit assay was performed as described in the manufacturer instruction (Promega™, N3030) and 15 μL of premixed detection reagent was added to the assay plate, incubated for 15 min and the luminescence signal quantified using PHERAstar® FS microplate reader (BMG Labtech). Data was analyzed using GraphPad Prism software.

Surprisingly, despite the potential competition for the same binding site on CRBN, combination of bifunctional compound 1 in presence or absence of dTAG13 at 1 μM concentration was only marginally affecting the degradation of BRD4_{BD1}L94V-EGFP. Similarly, the presence of 1 μM of bifunctional compound 1 had no effect on degradation of HiBit-FKBP12 F36V-KRAS G12C fusion. In both cases, treatment with bifunctional compound 1 or dTAG13 did not show cross-degradation of the reporters (FIG. 12B-FIG. 12C). We next tested the combination of bifunctional compound 1 with the VHL based degrader dTAG^{v}-1. Degradation of the HiBit-FKBP12 F36V-KRAS G12C by dTAG^{v}-1 was not affected by the presence of 1 μM of bifunctional compound 1. Interestingly, in a reverse experiment, degradation of BRD4_{BD1}L94V-EGFP by bifunctional compound 1 was enhanced by the addition of 100 nM dTAG^{v}-1, and this enhancement was more apparent with the addition of 1 μM of dTAG^{v}-1. The same effect was also observed with degradation-inactive negative control dTAG^{v}-1-neg, suggesting that it is not mediated by binding to VHL or by CUL2^{VHL} E3 ligase activity (FIG. 12D, FIG. 12F, and FIG. 15A-FIG. 15B). To identify how the enhanced degradation of BRD4_{BD1}L94V-EGFP by bifunctional compound 1 is modulated by dTAG^{v}-1, a dose response titration of dTAG^{v}-1 in the presence of 0.1 μM or 1 μM of bifunctional compound 1 was performed (FIG. 12D). 100 nM of dTAG^{v}-1 was sufficient to enhance the degradation of BRD4_{BD1}L94V-EGFP by bifunctional compound 1 (FIG. 12D). Intrigued by these findings, in the case of CRBN based ALK degraders, high cellular levels of the drug efflux pump P-glycoprotein 1 (P-gp), also known as multidrug resistance protein 1 (MDR1), lead to significantly reduced degradation of ALK, and in turn, pharmacological inhibition of P-gp resulted in increased degradation and improved anti-proliferative activity (Powell et al., J Med Chem 61(9):4249-4255 (2018)). dTAG^{v}-1 was hypothesized to saturate P-gp and, therefore, act as a P-gp inhibitor, resulting in an increase of the cellular levels of bifunctional compound 1, and hence enhance degradation. This is supported by the observation of similar increases in degradation potency of BRD4_{BD1}L94V-EGFP upon co-incubation of bifunctional compound 1 with 125 nM or 1 μM of the P-gp inhibitor tariquidar (FIG. 12G) (Roe et al., Bioorg Med Chem Lett 9(4):595-600 (1999)). Although drug-drug interactions may not be desirable in clinical situations, in the case of dTAG^{v}-1 and bifunctional compound 1, enhanced degradation can at times be beneficial as it lowers the effective concentration of the molecules, thus improving degradation selectivity. It is also important to note that no such drug-drug interaction was observed as a result of co-treatment with two CRBN based degraders bifunctional compound 1 and dTAG13, and target degradation was not affected by this co-treatment.

The structure of dTAG^{v}-1-neg is shown below:

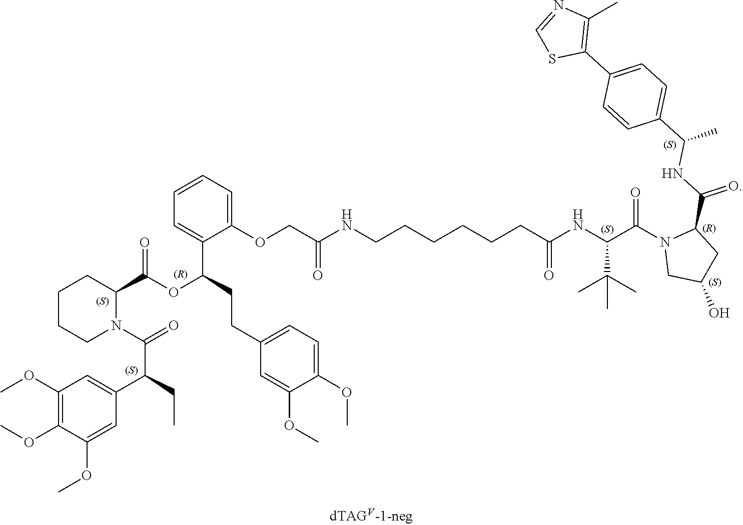

### Example 12: Stability of Inventive Bifunctional Compounds in Mouse Hepatic Microsome

An in vitro metabolic study was conducted at the Drug Metabolism and Pharmacokinetics (DMPK) core of the Scripps Research Institute, Florida. In brief, microsome stability was evaluated by incubating 1 μM compound with 1 mg/mL hepatic microsomes in 100 mM KPi, pH 7.4. The reaction was initiated by adding nicotinamide adenine dinucleotide phosphate (NADPH) (1 mM final concentration). Aliquots are removed at 0, 5, 10, 20, 40, and 60 minutes and added to acetonitrile (5X v:v) to stop the reaction and precipitate the protein. NADPH dependence of the reaction was evaluated with the addition of no-NADPH control samples. At the end of the assay, the samples were centrifuged through a Millipore™ Multiscreen® Solvinter 0.45 micron low binding polytetrafluoroethylene (PTFE) hydrophilic filter plate and analyzed by liquid chromatography with tandem mass spectrometry (LC-MS/MS). Data was log transformed and represented as half-life.

TABLE 6
Mouse hepatic microsome stability.

| | Half-life in 1 mg/ml hepatic | Intrinsic Clearance Mouse |
|---|---|---|
| | microsomes Mouse T_{1/2} (min) | Clᵢₙₜ (μl/min/mg) |
| Compound 1 | 18.4 | 38 |
| Compound 5 | 23.9 | 29 |

Compounds 1 and 5 showed excellent in vitro mouse hepatic microsome stability, with half lives of 18.4 min and 23.9 min, respectively.

### Example 12: Pharmacokinetic (PK) Profile of Inventive Bifunctional Compounds

Pharmacokinetic assessment was performed at Shanghai ChemPartners Co., LTD. In brief, compounds 1 and 5 (formulation: 0.4 mg/mL 5% DMSO+5% Solutol HS15+90% saline) were administered via tail vein injection (iv) at a dose of 2 mg/kg, or compounds 1 and 5 (formulation: 1.0 mg/mL 5% DMSO+5% Solutol® HS15+90% saline) administered via intraperitoneal injection (ip) at a dose of 10 mg/kg, to C57BL/6 male mice (Shanghai SLAC Laboratory Animal Co. LTD, Certificate No.: SCXK (SH) 2017-0012 2017 0012 006918). Approximately 110 μL of blood/time point were collected into K₂EDTA tubes via facial vein for bleeding for the time points: 0.083, 0.25, 0.5, 1, 2, 4, 8 and 24 h. Blood samples were put on wet ice and centrifuged to obtain plasma samples (2000 g, 5 min under 4° C.) within 15 minutes. Plasma samples were separated by centrifugation of whole blood and stored below−70° C. until bioanalysis.

For plasma samples: An aliquot of 30 μL sample was added with 200 μL IS (Diclofenac, 300 ng/mL in ACN). The mixture was vortexed for 10 min at 750 rpm and centrifuged at 6000 rpm for 10 min. An aliquot of 0.5 μL supernatant was injected for LC-MS/MS analysis. For 10-fold diluted plasma samples: an aliquot of 3 μL plasma sample was diluted with 27 μL blank plasma, mixed well to achieve the factor to 10. Then 200 μL IS (Diclofenac, 300 ng/mL in ACN) was added for protein precipitation. The following processing procedure was the same as those un-diluted plasma samples. The mixture was vortexed for 10 min at 750 rpm and centrifuged at 6000 rpm for 10 min. An aliquot of 0.5 μL supernatant was injected for LC-MS/MS analysis. MS analysis was performed using a SCIEX Triple Quad™ 6500+ LC-MS/MS system. WinNonlin® V 6.4 statistics software (Pharsight Corporation, California, USA) was used to generate pharmacokinetics parameters (CL, Vss, Tmax, Cmax, AUC, T1/2, etc) using non-compartmental model.

TABLE 7
Pharmacokinetic study with IV dosing.

| | IV (2 mg/kg) | | | | | | | |
|---|---|---|---|---|---|---|---|---|
| | CL | Vₛₛ | T_{1/2} | AUCₗₐₛₜ | AUC_{INF} | MRT_{INF} | MRTₗₐₛₜ | C₀ |
| | (mL/min/kg) | (L/kg) | (hr) | (μM * h) | (μM * h) | (hr) | (hr) | (μM) |
| Compound 1 | 21.9 | 0.378 | 0.515 | 1.88 | 1.88 | 0.288 | 0.283 | 8.40 |
| Compound 5 | 24.8 | 0.313 | 0.367 | 1.57 | 1.57 | 0.210 | 0.208 | 9.59 |

TABLE 8
Pharmacokinetic study with IP dosing.

| | IP (10 mg/kg) | | | | | | | |
|---|---|---|---|---|---|---|---|---|
| | Tₘₐₓ | Cₘₐₓ | T_{1/2} | AUCₗₐₛₜ | AUC_{INF} | MRT_{INF} | MRTₗₐₛₜ | F |
| | (hr) | (μM) | (hr) | (μM * h) | (μM * h) | (hr) | (hr) | (%) |
| Com- | 0.250 | 6.10 | 0.721 | 7.31 | 7.21 | 0.943 | 0.937 | 77.7 |
| pound 1 | | | | | | | | |
| Com- | 0.250 | 5.13 | 0.623 | 4.30 | 4.30 | 0.690 | 0.688 | 54.8 |
| pound 5 | | | | | | | | |

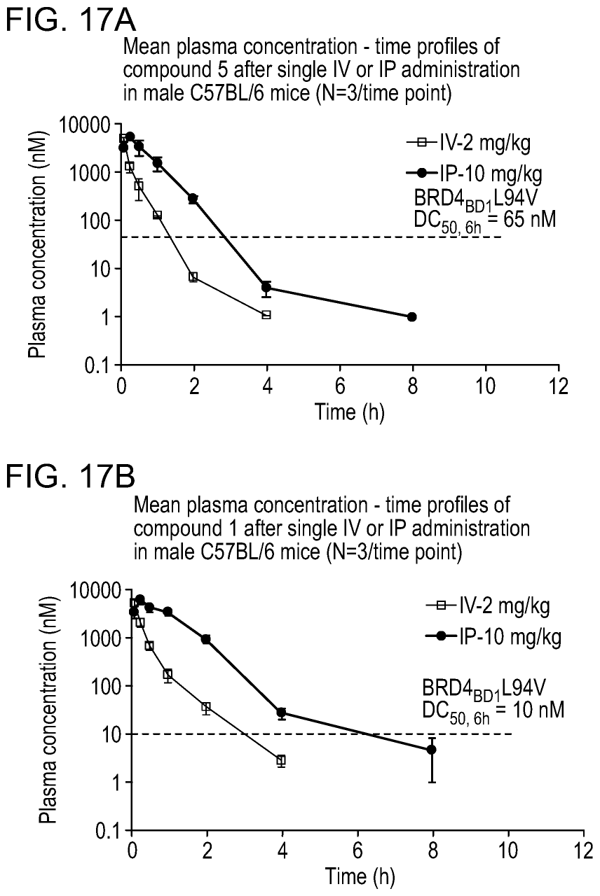

The data generated from PK studies in mice, which are summarized above in Tables 7 and 8, demonstrate that bifunctional compounds 1 and 5 had favorable pharmacokinetic profile, including good plasma concentration, and bioavailability. While the assessment of degradability of the BRD4_{BD1}L94V degron tag would require either establishing a genetically engineered mouse model, or a xenograft study with engineered cell line, the plasma concentration levels of bifunctional compound 1 was maintained above its DC_{50, 6h} of 10 nM for approximately 6 h when dosed at 10 mg/kg via intraperitoneal injection (IP) (FIG. 17A-FIG. 17B), indicating that such in vivo degradation experiment would result in a favorable outcome.

All publications cited in the specification, including patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention described herein has been described with reference to particular embodiments, it is to be understood that these embodiments are merely illustrative of the principle and applications described herein. It is therefore to be understood that numerous modifications may be made to the illustrative embodiments and that other arrangements may be devised without departing from the spirit and scope of the various embodiments described herein as defined by the appended claims.

1. A bifunctional compound which has a structure represented by formula (I): 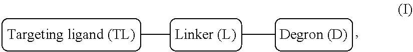
wherein the targeting ligand is represented by any one of structures TL1 to TL3: 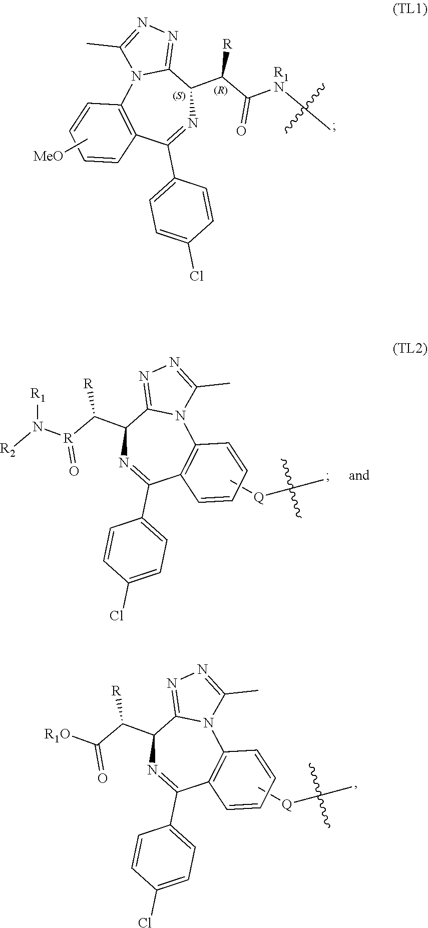
wherein
R represents methyl, ethyl, propyl, isopropyl, or allyl,
R₁ and R₂ independently represent H, methyl, or ethyl, and
Q represent a bond, CH₂, N, or O;
the linker represents a moiety that connects covalently the degron and the targeting ligand;
and the degron represents a ligand that binds an E3 ubiquitin ligase and is represented by a structure, or a pharmaceutically acceptable salt or stereoisomer thereof.

2. The bifunctional compound of claim 1, wherein R is methyl and R₁ is H.

3. The bifunctional compound of claim 1, the linker is a polyethylene glycol (PEG) chain which may terminate at either or both termini in at least one of —S—, —N(R′)—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C3-12 carbocyclene, 3- to 12-membered heterocyclene, 5- to 12-membered heteroarylene or any combination thereof, wherein R′ is H or C1-C6 alkyl, wherein the one or both terminating groups may be the same or different.

4. The bifunctional compound of claim 1, wherein the linker is represented by structure: 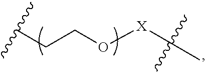
wherein n is an integer of 0-8; and
X is C1 to C12 alkyl.

5. The bifunctional compound of claim 4, wherein n is 0 and X is C1 to C12 alkyl.

6. The bifunctional compound of claim 4, wherein the linker is represented any one of structures:

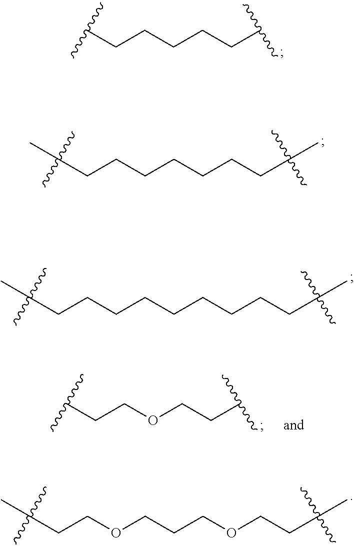 [SMILES: *CCCCC* ; *CCCCCCC* ; *CCCCCCCCC* ; *CCOCC* ; *CCOCCCOCC*]

7. The bifunctional compound of claim 1, which is represented by any one of structures I-1 to (I-6): 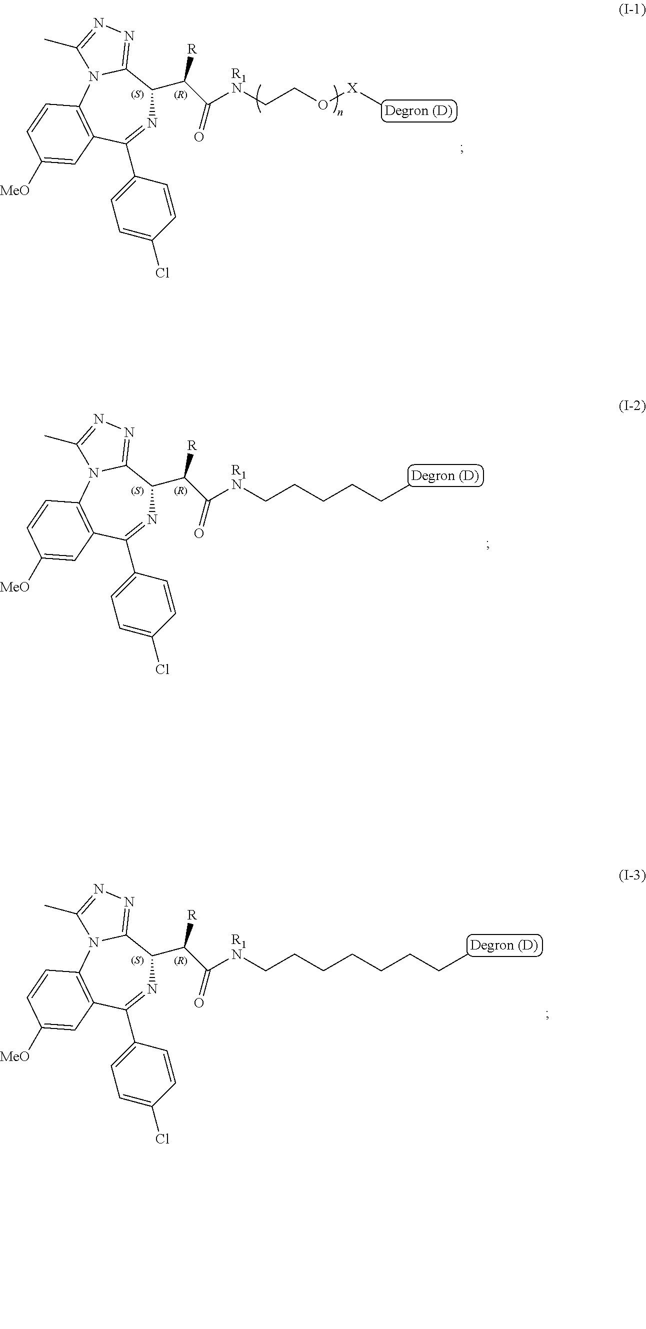 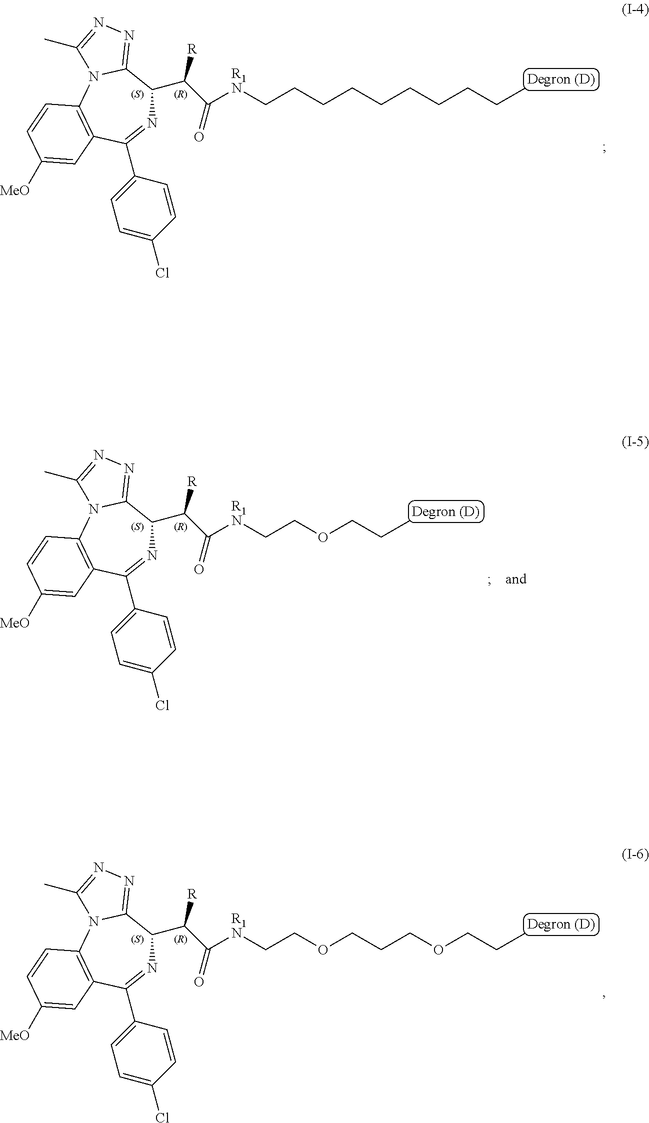
or a pharmaceutically acceptable salt or stereoisomer thereof.

8. The bifunctional compound of claim 1, wherein the degron binds cereblon (CRBN) and is represented by any one of structures (D1a) to (D1d): 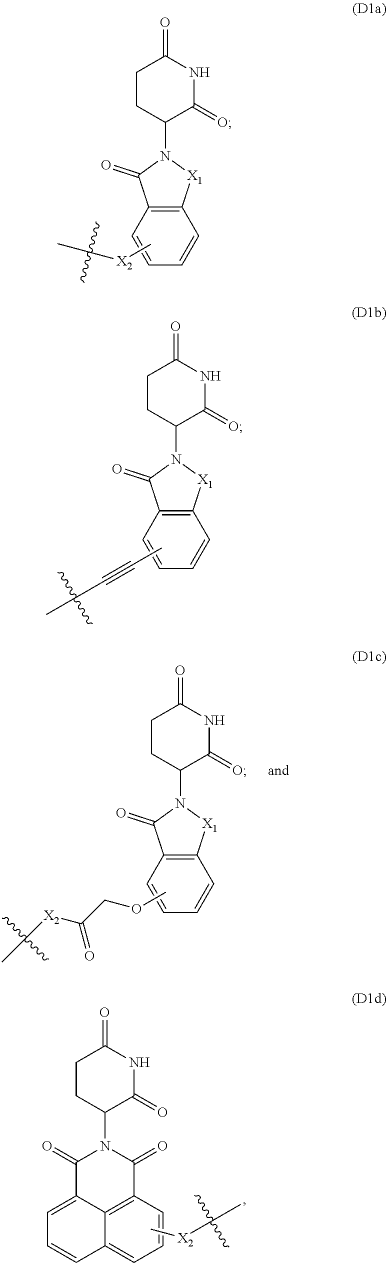
wherein X₁ is CH₂ or C(O) and X₂ is absent, CH₂, NH, or O.

9. (canceled)

10. The bifunctional compound of claim 1, wherein the degron binds von Hippel Landau tumor suppressor (VHL) and is represented by any one of structures D2-a to D2-g: 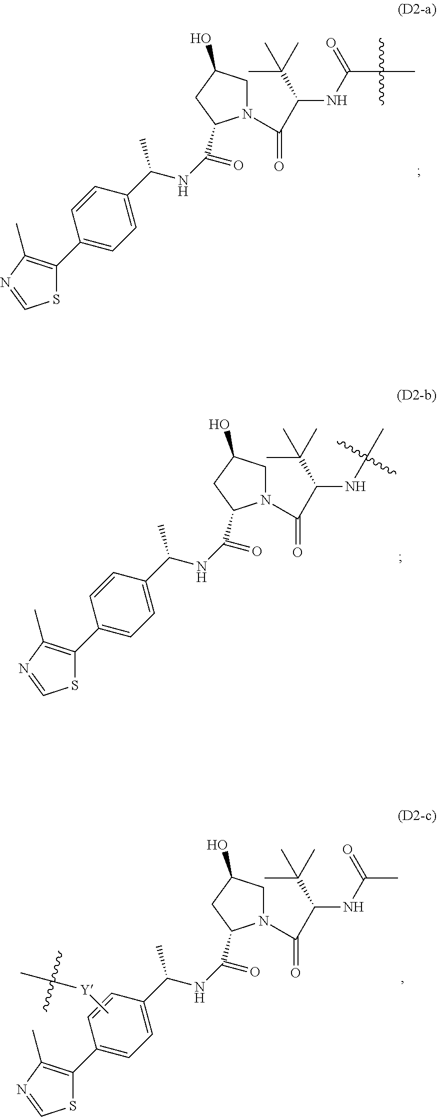
wherein Y′ is a bond, N, O or C; 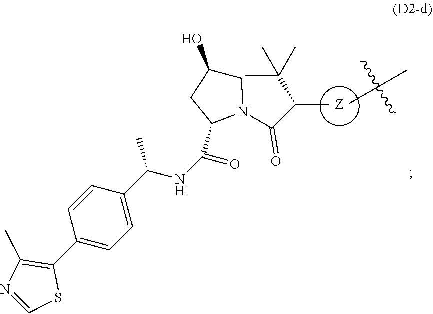
wherein Z is a C₅-C₆ carbocyclic or a C₅-C₆ heterocyclic group; 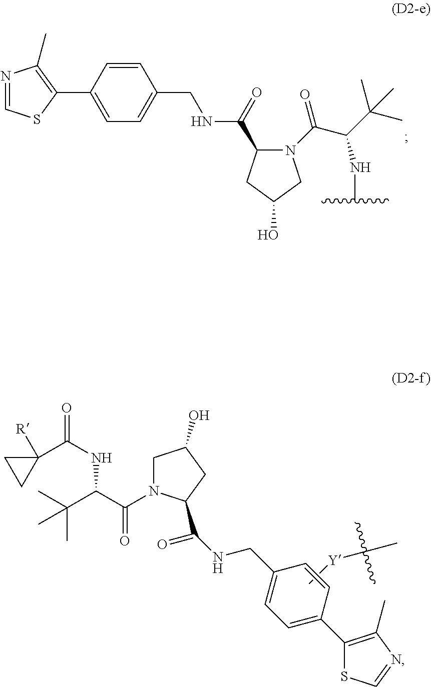
wherein R′ is H, F or CN, and Y′ is a bond, N, O or C; and 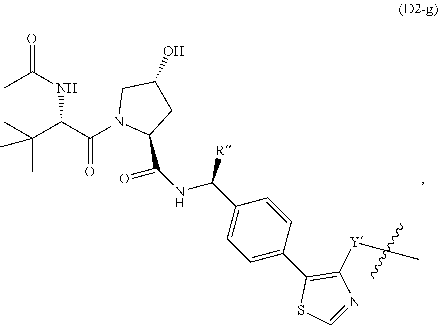
wherein R″ is H or methyl, and Y′ is a bond, N, O or C.

11. (canceled)

12. The bifunctional compound of claim 10, wherein Z is

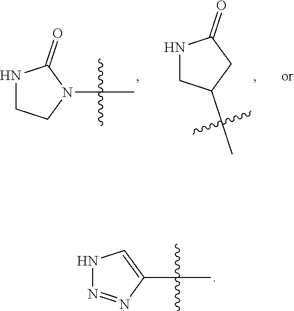 [SMILES: *N1CCNC1=O ; *C1CNC(=O)C1 ; *c1c[nH]nn1]

13. The bifunctional compound of claim 1, which is represented by any one of structures 1 to 5: 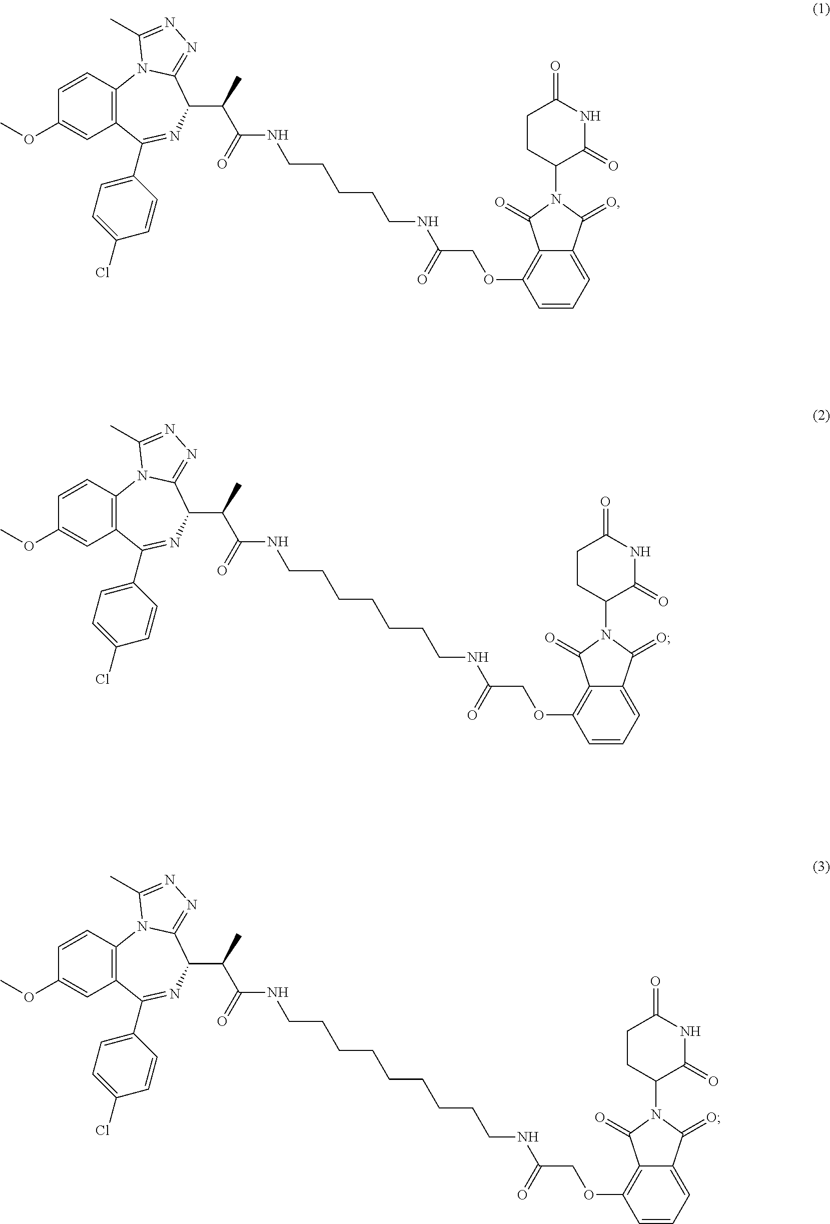 [SMILES: COc1ccc2c(c1)C(c1ccc(Cl)cc1)=N[C@@H]([C@@H](C)C(=O)NCCCCCNC(=O)COc1cccc3c1C(=O)N(C1CCC(=O)NC1=O)C3=O)c1nnc(C)n1-2 ; COc1ccc2c(c1)C(c1ccc(Cl)cc1)=N[C@@H]([C@@H](C)C(=O)NCCCCCCCNC(=O)COc1cccc3c1C(=O)N(C1CCC(=O)NC1=O)C3=O)c1nnc(C)n1-2 ; COc1ccc2c(c1)C(c1ccc(Cl)cc1)=N[C@@H]([C@@H](C)C(=O)NCCCCCCCCCNC(=O)COc1cccc3c1C(=O)N(C1CCC(=O)NC1=O)C3=O)c1nnc(C)n1-2] 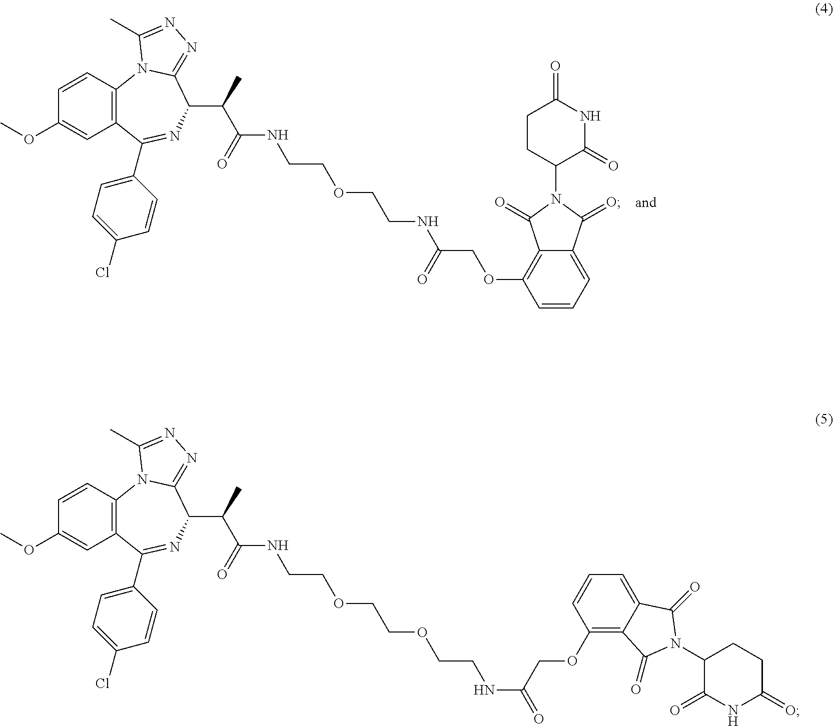 [SMILES: COc1ccc2c(c1)C(c1ccc(Cl)cc1)=N[C@@H]([C@@H](C)C(=O)NCCOCCNC(=O)COc1cccc3c1C(=O)N(C1CCC(=O)NC1=O)C3=O)c1nnc(C)n1-2 ; COc1ccc2c(c1)C(c1ccc(Cl)cc1)=N[C@@H]([C@@H](C)C(=O)NCCOCCOCCNC(=O)COc1cccc3c1C(=O)N(C1CCC(=O)NC1=O)C3=O)c1nnc(C)n1-2]
and pharmaceutically acceptable salts and stereoisomers thereof.

14. A pharmaceutical composition comprising a therapeutically effective amount of the bifunctional compound of claim 1 or pharmaceutically acceptable salt or stereoisomer thereof, and a pharmaceutically acceptable carrier.

15. A polynucleotide comprising a first nucleic acid fragment selected from SEQ ID NO: 9 to SEQ ID NO: 24.

16. The polynucleotide of claim 15, further comprising a second nucleic acid fragment encoding a protein of interest that is not natively associated with a mutant BET family protein.

17. The polynucleotide of claim 16, wherein the second nucleic acid fragment encodes a chimeric antigen receptor (CAR) protein, comprising, from N-terminus to C-terminus:
a) an extracellular ligand binding domain that binds a tumor associated antigen;
b) a transmembrane domain; and
c) a cytoplasmic domain comprising at least one intracellular signaling domain;
wherein the first nucleic acid fragment is disposed at the 5′ terminus of the polynucleotide, or between the extracellular binding domain and the transmembrane domain, provided that there is no disruption to antigen binding or insertion into the membrane, or at the 3′-terminus, or between the transmembrane domain and the intracellular domain, or between signaling domains when more than one signaling domain is present, provided that there is no disruption to intracellular signaling or insertion into the membrane.

18. The polynucleotide of claim 17, wherein said tumor associated antigen is CD19.

19. The nucleic acid of claim 18, wherein said a)-c) comprise tisagenlecleucel CAR or axicabtagene ciloleucel CAR.

20. A vector comprising the polynucleotide of claim 15.

21. A cell which expresses the polynucleotide of claim 15.

22. The cell of claim 21, which is an immune effector cell.

23. The cell of claim 22, which is a T-cell.

24. The cell of claim 21, which expresses the polynucleotide of claim 15 which is positioned in-frame with a genetic locus of an endogenous protein.

25. A method of identifying protein function, comprising genetically modifying a cell by introducing a polynucleotide comprising a sequence encoding a BET protein, or a bromodomain thereof containing a BET-ligand binding domain, wherein the binding domain contains an amino acid substitution of a conserved Leu residue (dTAG), in-frame with a genetic locus of an endogenous protein; contacting the thus modified cell with the bifunctional compound of claim 1; and detecting a change in a property of the modified cell relative to an unmodified cell.

26. A method of identifying protein function, comprising genetically modifying a cell by introducing a polynucleotide comprising a first nucleic acid comprising a BET protein, or a bromodomain thereof containing a BET ligand-binding domain, wherein the BET-ligand binding domain contains an amino acid substitution of a conserved Leu residue (dTAG), fused, in frame to a second nucleic acid which encodes a mutant version of a protein that is endogenous to the cell; contacting the thus modified cell with the bifunctional compound of claim 1; and detecting a change in a property of the modified cell relative to an unmodified cell.

27. The method of claim 26, which is conducted in vitro.

28. The method of claim 26, which is conducted in vivo in a non-human animal.

29. The method of claim 28, wherein the non-human animal is a rodent.

30. The method of claim 29, wherein the rodent is a mouse.

31. (canceled)

32. The method of claim 26, wherein the cell is a human cell.

33. The method of claim 32, wherein the human cell is a human cancer cell line or a non-cancerous cell line.

34. A method of degrading a chimeric antigen receptor (CAR) protein comprising:
administering to a subject an effective amount of the bifunctional compound or pharmaceutically acceptable salt or stereoisomer of claim 1,
wherein the subject has previously been treated with allogeneic or autologous immune effector cells that express a nucleic acid encoding a CAR and exhibits an adverse side effect as a result thereof, wherein said CAR comprises, from N-terminus to C-terminus:
a) an extracellular ligand binding domain that binds a tumor associated antigen;
b) a transmembrane domain;
c) a cytoplasmic domain comprising at least one intracellular signaling domain; and
d) a BET protein, or a bromodomain thereof containing a BET ligand-binding domain,
wherein the BET-ligand binding domain contains an amino acid substitution of a conserved Leu residue (dTAG), wherein (d) is disposed at the N-terminus of the CAR, between the extracellular binding domain and the transmembrane domain, provided that there is no disruption to antigen binding or insertion into the membrane; between the transmembrane domain and the intracellular domain, or at the C-terminus of the CAR.

35. The method of claim 34, wherein the CAR comprises at least two signaling domains and the mutant BET family protein is disposed between the two intracellular signaling domains and provided that there is no disruption to intracellular signaling or insertion into the membrane.

36. The method of any one of claim 25, wherein the dTAG comprises BRD4 bromodomain 1 (BRD4_{BD1}) having a conserved L94 substitution, BRD4 bromodomain 2 (BRD4_{BD2}) having a conserved L387 substitution, BRD2 bromodomain 1 (BRD2_{BD1}) having a conserved L110 substitution, BRD2 bromodomain 2 (BRD2_{BD2}) having a conserved L383 substitution, BRD3 bromodomain 1 (BRD3_{BD1}) having a conserved L70 substitution, BRD3 bromodomain 2 (BRD3_{BD2}) having a conserved L345 substitution, BRDT bromodomain 1 (BRDT_{BD1}) having a conserved L63 substitution, or BRDT bromodomain 2 (BRDT_{BD2}) having a conserved L306 substitution.

37. The method of any one of claims 26 and 34, wherein the substitution of the conserved Leu residue is a conservative amino acid substitution.

38. The method of claim 37, wherein the substitution is a Leu→Val substitution.

39. The method of claim 38, wherein the dTAG comprises any one of SEQ ID NO: 9 to SEQ ID NO: 24.
